SEQUENCE LISTING (19 sequences)

SEQ ID NO: 1 - DNA, 47337 bp, Homo sapiens
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtaaggagcccactctggaggaggaaggcagacaggtcgggtgagggcggagaggacctgaaagccagatctaactcggaatcgtagagctggagagttggacaggacttgacattttgcgatctttcatttaccagtggggaaactgaggctcagagactggcccaagattacccagcgagtctgtggtcgcctgtgctctagcccagttccttttctaggactctggtttgcgacagggacctcggctggagcatgtcctgagatgccgacacaccctcaggctcttgggaggctggggtgggaaggcgcctggggttggcaggcaggaggtgcctccgcaggcgagaacaggcggtgaaaagttgtctggctgcgcgcaacatcctagtccgggcccggggaagaaaaccttgccggaatctcaggccgggtctcccggatcggacggtacactcggttctgcctctttgcgggacccggcccgttgttgtcttcatgctcgaacacacttgcacaccactgtgtgaagtggggtctggagcggagagaaactttttttccttccttggtgcaggacgccgctctccttgcagagcgaagaagggggggaatagggacttgtcctgggggctttgacagcttccccaagggtctccaagtaacagccaactgtcctgcgtaaagcattgcacatctttcaaagcgctgtggtccttggtgtaagcgcatagtcagaagttcaagctccgaaaacctttcctgtgggccttggtacctagctttagtgccattccttcctctccctgccgcctaaaatttccgtctccttcaattaggaacacacacgttcttcatgcaatagctgtctgtcttttcttcctcactttcctttctctctcaaccccttagataatatttctttcctgcagccagtttgctgatatccagatttccaccctttgcagggtgagaaaggggaaagggtcagagaaagaaaaaaaaaaagtcgaataattcagggaaaaaaatttcttactccctaagacaagaatcacatgtcttagaagacactcacacccacatacagtaccaggatcatctgtccatggttactgaattttctttataatgacttggttcaacgggtccagtccaccatggacactcatttgtcccagacaagccctctctctccccctttctgggcagagaatgaaggtctggaacatgtggttgctctgtattccacaaagaagtgagttgcttttaagcctggggtgtttcctagcgtagtagtaacggcaggccggtcgccctgaatataatggtgaacttgcccttttggagtgcattacttgcttaattggattgggctgtaattggtgccatcaaattctagagacagaggcactgttgtttttccttcccgtctttgagctggaagggtaacagtgcacaaattaattaatattggttatgggatttgaacatagaagggctttttattgagtagtagcatgtgtacctcttacagttatttctttagaactttctgaagagtccagctcaagcttgccaatgaaaacgaatgacatttaatggagcaaaaacaaaaaacaaaaaactatgttggtctacaaatatgaatttgaagttattgagagccttgttgaatagatttttgttgtaaacgtgtctctagaatagtatggcatagtctcagcttcctatgaatgaaggacataccttttcttttttaaaatatttgttacacaggaaagtgtgtctagaatgtgatctgtggcaataaattatgagagaccttcaagagtttctgattttggtagccgagtgggcacagtttattgagaatcatttttactgccatttgttttctcacaagaatgtgcccaaataatggtttttttctcatttggatggcagtgtgaattgtacatcatgttttcagcatctttctcaacctagtgttccccagtcaagtttgaaatctgtgttatccaaatgaattgttttcattttccttttcttagacaaagtgggactccaggtttcattttgcttttaaacattttggttttttgtttgcctgttttgggggcagttatttctttcatattaaaaagtactgtgcaggctgggtgcagtggctcattcctgtaatcccagcacttagggaagcagaggcaggaggatcgcttgagtccaggagttcaagaagtgcctgggcaacatagcgagaccccattctctatttaaaacataaatgtaacccccgttccacgcacaaagtactgtgcaaattaattaaacatgaccacccagaccagcaactgtccaagagtggcccatagaccatctgtggtaggataatttgaaatgcttgttaaaatgcagatttgtagacccagggatattctgacagagtctaaagtcttaagaacaaaactgttctaaacataagtcagtaccaatgccagttaatttctgagatatattgatataacttagtttccagttttttaaaaaccatattattgacttaaaaaccatgatattgaccagttatgtcagtaacttattttgcacatctgtgtggtgtgtgagaacatgtgcagtcacttattcattttgcctgcatttgttcatattgggatcctcagattcaatgcactggatgtttgcactgggtatttacttatactctctctatttattccgtctcatacttcgtcctatttgttcatactctcttatttgcccagcaaggtcaatgccagtttaggcctagggagtcattttttcttagttgatatgacttagaaagcttgggagcctgcccaacatcaattacttttttaaagctggtattttctaggtcttgatatttattaagaccctagcatagtggacaatttttctttctctcatgctttttcaacacctcatagctcttcacatttagttgacagagaattcagttatcttgctgtagagtgacccatggtgaggaatctatgccatggtacttttctggttcttatcccttataggtaaagacaagtttcttatgtctgaagcttgatgtcaggatgagttcagggctttgatgaataagttcagatctcccaattgtaattcattagcattgcacttaaaaaaatttatatacgtttttaaaaaagggtaatgctaatgaattacaatagagagaaaagtacattagtttgcatgtatgtgtgaaactgggaaaatttttcacgaaaatattcatatactttttaaaaaaagggtaatgctaatgaattacagtagacagaaaagtatattaatttgcacatatgtgtaaaattgggaaaattccacacatacataaaagtatattaatatgcatgtatgtgtggaattggggaatgttttctcttcctcagtttctctcccttgcttttaatgtacagtctttatgagccattatttcagctgtggcagtttggttaccaggggaagcgcactagaaaattgataaaggaaaatgagacaaggtcatagattctctcactcccttcagggtacgtagatgaactatataaaaatccgtctaagtgggattcgttaatcagcaatttagtcaaatgtgtacatcctatgttctataagaaatgtcagtgggtcctttcccaagggagtgagatcatcagatgaaggttcatttggtttcaatgtcccgtatccttttgtaagaccttgaagttggcaatgcaggaaaacaggaactccaccctagctccatgaattgcagaactgttgtgttggtttatgaccatctgcccattcttcctgttatgacacagcttgtgaacttttactgagaatggtgaaaagtaaattcccagttttatacaatgaattgctgaagaggccttttaaagtatagagtatgcattgtttatggaaggtgtttcctattaggtctaactcagtggcaactacattcatttatttaatttgtttctaggtttgcctagtgtttctcttgatctgcccaggctcagcatacaaaaagacatacttacaattaaggctaatacaactcttcaaattacttgcaggtaaggattcattctagatctagatttcttgtgttaagtaactgattgtttattgagtggaaataatttccagtagagcagaattataatagagcttgtagtaattgttcataagtggtgaggtttctaagaactgatgtaataatggaaaatgagaagaattttctctcaaaaattctgtacaattttgctggtgtttttatactattctctgccaacatgcatacacacacacacacacacacgcacacaaatacacacccacacccacattccaataaccagtacagccacctggcgtatagtagacatacgctcaataaatatgaatgaataaatgaagttgagggcatacatttaaggaatagagttgaaaaaatttgggactatatttattatgcttggtatgattcttgaacacttattatccctttccaaaaactttgctttataagaaatttattactataattacttaggcagtaatatttaatagcaatttaatatttagtgggtaatattactgagcgcatgatctacataaataatggacttcgggccctgccttgatattctggaatgcatctttccccacttgctagcaagaagtcatgctattgatttttgataactggagaagtagacttctttgtcaagaagaagaggcctttaaattttgcctttcaacccttaccccaggacgaaagatagaagacccttgggtttaacatagtgatcacacacgaaaggcatggagccttcttaggacctgtgtgtttttggtagagactgtgacaagtggaggtgatgttaccctcctggaagagtgctgggggtccacaaaggaccttgggtaggttattgccattgcttcatacttgttgaatactaagcattaaaccgaatgacatacatctattttagactgcagtataaagaataccctagccccttaccaatacccagcccttgggaaaaaacacagtagcaggtgctgtttctctagctttacttgtttaagacacatttcccattagattttccttttaccgaccctcgataacaaggttatttgaaatccccaaggatcccatgctccctttttaaaactctgcataaacatttcttatgttctgaaaaaaaccatggagtgtgttaaaagtaacttcattgatttagctgcaacttcctggaaattttaagttctttgaatgaagggccaataatgttacattcttcttgatgttgactatcttcttatcttccttggggccttgtagagaaatgctgcagtacaagccatctatgttttaatgcgaggtccttacaaggtcctgagggactcttacttgcacctccttccttcctaacctcacttcttactcccctttgctcactcttacctggctgctctggtttcctggctgttcccttaatactccagatatgcacctgctccagggcctttccatgtgctgtttttgctcctgtaatactgctcttcatgatgttcctatggctagctttatcaagaccacctcctgcaaaattctttactcttttctttgtatcttctatatttttctccatagtactaaacactatcttttatacaataaactttccttactttttaattgcctgttttctccagttagactgaggttccataaaggcattgatttttgtctgatttgttcactgctctttctctagtccttaacaagtttggcacatagtagatgcttaatagatatttgttgaaagaaagaatgcattaattaatggaaaactcaggaatctttataagtgacttctgaagctgagtttataacttttcatcatatgtcaatctgacttgttggtagaagactttgtttttttttttttgaggcagggttgccctcttgcccaggctgaagtgcagtggtgtgattttggctcactgcaacctccacctcccgggttcaagcaattctcatgcctcagcctcctgagtagctgggattacaggcatgcgccaccacacctggctcatttttgtatttttagtagagacagggttttaccatgttgcccagcctggtctcgaactcctggcctcaggtgatccatccgccttggcctcccaaagtgctgggattataggcatgagccaccatgcctggccggtagaagactgactgtgtctgttgaagagtttatttaagtttcaaaaccaaattttctcttttcttagaaatagcctcacagtctggcacttcatattaatacctccctgaaattaatttttcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaaggtaagtggtgaaataaaattcatttcccacgtctctttaccagttataaaagacaataggctcaaagaagaattgagtacaacaaagggcttgctctaaaggctgtttgccaagaggaatacacacaattcttctctcctgaggctttctctgagaaataagactcattgattctggagcttgggccgtgttacctcttttttgcccagttagtttgggtctgatctttgtttccaaggtaaatctgtgttcactgttggccattgagacttataaaaagtcttcctatgtttgagaagaaaacctaaaattcttgaaatcgaggaagatttgggggtgaattatggagaaatttctgtggagagataagttatctacagcagagtaggagattttcccaagaatgcataggaaagcattttttgccaagggctctggagttttttgcacataggaacctttttttcttactagtatttcataaaaaacaattcccatactcatgtgcaaataaagacattgcttcagactcttttcaggacaatgtttctttcctttgcttgtttggtctgagatcttggatgatatgctgtatctttctaggatgtgcagtttgggattgatattatgaaggctgacttaacatccatatagtataaaataaatgtcacacatattctgcatttataatgagttatgcattcttttgtgtttcaaaaatcttacactatcttatcttttctgtgaaaacctaacttaactaatgagatccctatgatataaatttaaggaatgtaagggctgcatcatagtttggttggatgtaccaaatatttttcttttcagtgaagataaacagacattttatgtatttacgtatatgcctttttacatcccagagtatttgagacaggtgaagatgacttagacttttttcccagaagcagcttttacagggcaagaatttcatcagctttgggaaacacacttgcatatctctgcttacatttcagtagtgtaatatggtcagtgcaatgaaaaagtggagaccacatcaaaataacctatgccactggattcacaatgtttgagaaatatctttgcccagagtaagcactgtcaaagatagaattctgtgccctcctccttccctccacaagatttgaaagagacaaggctcacatcttggagaatttctggctccttttgacctggcagtcttgagagatgcagctcggtcagaagattgcaaggatttcctgctttcagcctgtctagaaatactacaagatgaacatcccccatatctcattatttacttcttcctaagtcaggaaacttggagacatgtgaaaattcatttcatgagtttcagtaaatattttattttgagaggctgggtggtggtttgggtttcttttgtttatttcctttttttgagataccgaaatagaattgatttactaaataggtttagtcttacgtcaaagggttaatttagcttccaaaggcttgctctgtaagcaagttatgtaatatttcataacatgtggatgaaaggtaggcaatattaagaagtggcaatccctagcactgtttattggtacactgcctgtctttgggtataccattaaattctgcttcctgtctaagcttaaagttctaggagttgggctgtccaagattttggccatgaagttaaacaatgggaaaggaaacactgaagtattctctatggataggtgtttaatgtcccctctggtcgccaccttacttccctagtcttctgaccccattctcttcagcaatggatggagccaggaagtgagccctggcctcataagataatggctatggcatgtggtgggctagattggctgcttttctgtgctttccagctgggaaggaaatcaaacttctgctgttgcagggaattagctgcctttgtcccctgtggtttaattaactctttcttcactttgactgactattatgaagcactctgagaatgcttgatgggatgtgttgggcatagcaatgtgaaatgttatctctctgagatttcaagcatgactccacaccacatcatctctatctctgaggaatggactaggtttccagcagcatgttaacattgtatgagtaatgtttgattggccttgaaatctttttttttttttttttttgagacggagttttgctcttgttgcccaggctaaagtgcagtggtgctatctcagctcactgcaacttctgccccccggttcaaatgattctcctgcctcagcctctgaaatagctgggactacaggtgcgtgccatcatgcctggctaattttttgtatttttcgtagagatggggttttgccacgttggtcaggctggtctcaaactcctgacctcaagtgatccacctgcctcagcctcccaaagtgctgggattacaggcgtgagccaagaacccagtcagaatctcttcagttttcttctcagtctttggagtggtgacttttcaaatgtttgtcattgaagatatcaatgactgctaaatgttaaactaaatgcaaaaacaattaaacatggttttagaaagaatcatatccctagtcttcagaatcttaaaatgctcacatgaatggtcctcttgaataaccaaattcaaaagtgttagctgtttcctgttaatctaaagatcctttgggatccattcatttattttcatggaatttacattatttacctaaagagagagcacatgagtattttaaatattagtaaaacttgtcggtaaagtgtatagatttaactttaaattttaaagtaaatattatccttcattttgaaaaaattataatgattaatcttttaaaatgtgaaatctataaaaatatattctgcttgtcaataaaccttgtgaaaggagtcaatctcaattgggagttttttttcaaaatttttatacacacagatatatacacatgcatgtgcatgcacaaacacacacacacacatacacacacaccctcatgtagcacagatatctatcagcagaataatctgtggatgcctttggttgtgtgaggtgtcccttccagtcattcacttgtctggttagagtttaggaacctgaaaaatgaccaacttttctagtaaatactattaactcattaataaaactaaattttcttctagattacagatctccatttattgcttctgttagtgaccaacatggagtcgtgtacattactgagaacaaaaacaaaactgtggtgattccatgtctcgggtccatttcaaatctcaacgtgtcactttgtgcagtaagttgcatctcctccaatcgtctcttaagtttttataattttaagctaatattaagatgggtaacctgtttataatattcacaatgagttttaaggatcctttaggaagggtcaaatgcaatgaataaaactaattagtattcttaaaaataagatgaattcttcagtgatcattgtacatggctctcatttttggtactggattaaatatttgatatgtctttttattacccagagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtaggtaagaggacatttcctttccatatcattaataacatatccttgtattaagatcttggagataacaacatagagtgaagaaggatattgaaaagtataggaactcaggatatggtgttgggcaattcatctgctcttctctaccaaataaacccatgtgcaattgaggttgtctcttttcttgccaagattaaggaagaaaaagaaaactttttaaaaaaaggatgaaagcgaatggtattactcgagcacattttatgaagaattcaatgttcagagcattgcttgctatcaattatttcaattatgactattttatggaaacttcagcaatttgctaaagctggccctactggcctagggctactgaccactgaaagtttactacttttctgtccactgggttacaacatctttgagatctgtgaaggtagtgctttgtaaacctctgttggccattttcctgggagctaccaagtattggtgaggcctgcagggaaaaacaatgtggcatgttttaaagttgcattactttaaaaaataaatctgtgcaaagttataggcttatttgctctctcatgttctgttttttcaatttacttgctctagggtataggatttatgatgtggttctgagtccgtctcatggaattgaactatctgttggagaaaagcttgtcttaaattgtacagcaagaactgaactaaatgtggggattgacttcaactgggaatacccttcttcgaaggtaacgctaatgattcaaagccagacctccaaatacttagataataagccccagtgaagtttgcttgagagataggggcctctttggccagataaaatgtaagagccttaaacacacacacatacacacccactcacacacacatacacacacacacaatttaagggaattgcagaacagatagcacccaccaaaaggtgaaataccaggaattttgtcctattctgcaatagccaggctatgaatattagttttctctaggtgattacatctttccacattatgtcatttctctgttctccaaagtttttgatctacattccttttaagggaatttctctttaagaggtggcatgagatacactgctccttaaacagtggtcacatttacttgtgtttctgcagtttatatccatctcactttcaccacgtgaggttttaaaaatcctaattcagttggttccatttatttctcctgaaacaaaatatatttgttgtctgcatgaggttaaaagttctggtgtccctgtttttagcattaaataatgtttaccaaagcccagatttaattctgtgtgttactagaagttattgggtaatgttatatgctgtgctttggaagttcagtcaactctttttttcagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatggtaagctatggtcttggaaattattctgtgccttgacaagtgagataatttaaataaatttaggtcacttagtgattcctattttgttcattcagaagatagtttctagtttttcttgttagggaggccacatgacctagaggtcaagagcatagctttgtagtcaggaacttgggttcaaacctcaactttaaagatgagatgtgctgatatacagtaagagttcatttagtattacttattatagttattgctgctattaggattgttactatgataaatagtattagctaaggtagtttttaaattttcattttattgcaaggctgagaggcctacttgaataagcatgagctttgcaaactggggaaacatttagcaatatacagttgacctgtgagcaactcagggattgggggaactcaggggagttcccctaactttccctcctctgcagtcaaaaatccatgtataggccgggcgcggtggctcacgcctgtaatcccaacactttgggagtctgaggtgggtggatcacctgagatcaggagttcgaaaccagcctggtcaacatggtggaaccccatctctactaaaaatccaaaaaattagcctggtgtggtggtgggagcttgtaatcccagctactcaggaggctgaggcaggagaattgcttgaacccaggaggtggaggttgcagtgagccaagatcgtgccattgtaccccagcctgggcaacaagagtgaaactccttctcaaaaaaaaaaaaaaaaaaaaaatcaaggtataacttttgacttccacaaaacataactaatggcctactgttgactggaagccctactgataacataaacagtcaattaacacatattttatatgttatatgtattatatactgtattcttccaataaagctagagaaaagaaaatgttattaagaaaattgtaaggaagagaaaatatatttactattcattaagtgtaagtggatcatcataaaggtcttcatccttgtcttcacgttgagtaggctgaggaaaagggggaagaggagggggtggttttgctgtctcaggggtggcagaggtggaagaaaatctgcttataagtggactcatgtagttcaagtttgtgttatttaagggtcaactgtaattgaactggaattaaattgaactggccttgagaaaatcaccttaattttttgtttattctctttcatttacataaatgtctgagtttacatggtaatttgtgtggcatcctacttataagccttggaaaggattttggagtttatattatgagaatgcatcaatacagtgaaattttaaaaataccttagataatgctatttattagagttgtaatcataaaagtggcaacaactataacaagtatgatttagtgagcacttactttattagctcatctcatctttgaagctgagattggaactcaagttcctgactacaaagctatgctcttgacctctaggtcacgtggcatccctagcaagaacttgaaaatttcttctgaatgaacaaaatagaaatcactaagtgtcctaaatttatttaaattatttcacttgccaagatgcacttgtcaaaatacacagagagagatgtgctctggcttatgtttttatagaattacttttgttttccagaatacttcagggaaataggggcagaaataaggaggtcagttgggaggctaattgcagttatccaagtgagagttgaggggtggcttagacaagggtagttgaggtggaggtagtgagaggtgatctgcttctggatatattttgaaggtagagtcaacagggtccgctgatcaattcattggttgtggagtataagagaaaaagagtggaagatgactcgagcgttagcatgagcaactgagtaaatgatggtgttatttactgagatggcaaagatcgagaaggcagtgagatttagggaaacagtgttagatatgtttatctggagatgcctgttaaacatccaagtggagatatttaacatatcaacccggaacccagaggagtcagggcagaagataacacatttaggaggtacgtgaatgatactttaaacctgaggctagaggaaggtgtaaataaagaggaggtctgaggactgagtcctggggcctcatggtggaagaggtgtgtggaggctgtcatgggagcagaggagaaggagcacccaagcatccctgggggacttagagaaagctgcacagaggagcaagtgtttgagttgagacttgagcaatcactaggcttgtgggagtgcactagcggggagagaaaagcaaatgcaaacacaggaggtgtgggagaaacacgggaggtgtgggagaagctgaaaagtgacccactgaaagatagtacaggaaatcttggaactgcagctactcagaccctcaaggtctttgacgtttcacttgaaatgaaaaactaaatcaaatgaccatttacagtaagttgacctttttttttttttattttcttccagaaaaaccttttgttgcttttggaagtggcatggaatctctggtggaagccacggtgggggagcgtgtcagaatccctgcgaagtaccttggttacccacccccagaaataaaatggtaactactggaaataaatgcaaagcatcatttcgtgtgagagcaaatcctttgactatactaattcctgagaattttttttcataggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatggtgagtccattcaattttcctctctgcccaagatttattatgatacattgtcttccaaatcagccaaaccaccgttcctctgcctcctgctgcttcactcatatcatggctgggcctgcgtacaaaagtcatctggcgtggtgaagctgaagtgaaacgtaggaccatgtgctctggccatgtttgtttaagaggccgtgtaaatgagctttgtggtggacaaatgcaagattaaagtagtgataccctcgatagctaaatgttgtgaaataagaatgcccacagggacagttgtcaagctaagttatactaccatgttcccctctcatggaattgcccacctggtacacagatgtgtaagacccttctccttagattttgtgcaaagcttctagtttgatgttgtagttgatgtatcagagatgtgcaggcacgttccaactctgaaggcttttgaagttgacactgttggcttggttgggagcttttcttttttcctttttgacaggagttcaggatctgattttgagtctgtaaaggaaagatagtaagtttttgatgtaaagataatttgaactttgttttctgaaactgaaaggtacaaataagtgtttggaatggagtggggagaagggtgccatggtcaagtgagtgtgagaggtgctaaggtgatgtgtagatgtgtaacaggtttctttattgcaggacttcgcagaaccttttatatgctaatgtatattggtattctccaggaggagagacatagagtattcaaggtttaacaaacctatttgaccagagcaccttttttcccctgagcaaattcattaatctctcactccaaacagtttgagaaatgcttctctgttgtaattctttgttcccccttctggtacggcatattaaaacttcaggatattttcccatgacattaaggtgcttccctacgtgtcctgatactcttctgtaggccgctgaacttggctttattattttttttcagggaatattttaaagataggctgggtgccgtggtttgcatctgtaatcccagcactttgggaggccgaggcggatggatcacctgaggtcaggagttcgagaccagcctggccaacatgatgaaaacccgtctctactaaaaatataaaaattagccaggcatggtggtgggcacctgtaatcccagctacttgggaggctgaggcaggagaatcacttgaacccaggaggtggaggttgcagatagccgagatcgcaccattgtactccagcctggtgacaagagcaaaactccgtctcaaaaaaaaagttaacaggttccaaaaaggttgtttagaagcagcataggtgtaggggactggggagaggagaaactggaaagtgtataagtaggatgggaggaggaaatgaacaggaaataaaaacaaaacacggacagcaaatagcccatttcatcagttcatgaagccactaaatattttattcactttagcaaattctctgctatatgaaataaacataaaaaagaagtcaagtcttcaaagcataatctgaggctttaggttgacagtaataaggaaatagttttgactttggagtcaaaaaagaaagaaaggaaaaagggagagaagaaagaaggaagtgagagaagggagaaggaagaaaggggaagagggaaagggagtggagagggagggagggaggaagagggagagagaatgaaaaactcagatgatggtggcaggaatgcattctctaaagatttacaccttcctttaacatgaggtggtttacgtgtttgggttcagaagtcagagtgtctaggtttgttccaggttttgccgttcgttaactgagtgaccttgggcgagtcatttttttctgtttcatttttttctcacgtataaagctgtggacagtaatagtggttgtgaggattaagtgaatgaattcatgcaaagcacttcaaacaatgcttggcacataataaatgtatttactgtgctatttcagctgttttctgtagcctttccctgatctcctaaacttgagaggacagagagaactatctctgtaatacagatgagaggcacaggatttcaacacttccataaagtcattcagcttgttagtttattattattattagcttattgtcatttttattttatttcgttactttattccttttttttttttttggtagagatggggtctcaccatgtggcccaggctggtcttgatctcctgggcttaagcgatccacctaccttggcgtcccaaaatactgagattacaggcataagcccccatgcctggctagttgttatttttatgagtatcactagaactcaggtctcttgtttccacatctaggtgttcttcgaaaaagaaagtggaagcaaaatcatatgcttaaagaaagtcagctttagttgctaaaatcctctatttcccattcttcaaagctgactgacaattcaaaagttgtttttcccatcttcagtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccaggtgagtaaggccacatgctctttgctttcctgccatcttgcatttcttacagctgagctatgatatgactccatcctaaatggagaagcctaaaccaaaaaaagttttctctcaagaggtagcctgaatctccatccatctttctctgtgtcttacattttaggggatgtctttgcttggagtatcctcctttggggttagctaagctcagccttgttaggttagccgtgaggtacacttctccaaacacaggctatttgctcagtttgctaattgccagtctttggtttttctcccgataccaatcggctggtgaataccacatccctccttcttgtgtgtgtgaagatccatctctcagaggaaatgctgatagatgagaggcagtgatagacccagccccagtcctcagggtctcaggcccagcttatcatgctctgacacaagtccagacatccttagggaaaaacacaacaacagcagccaacccaccaccaccctaagcagtccacttcctgttgttgtttttgaaatggccactatgagcttcttcctcagctgctgatcatttccttcacagagaccatggtcccagagaaattactttaaggagcccagtggcttctaagtttccttgccttcctttgaactaaattaacttgaattgtcttgtcgatccaatttatgaatgaaggtttattcccagaatagctgcttccctcctgtatcctgaatgaatctacctagaaccttttccttcattgtcaatgcctatttttaattggcgccaagtcttgtaccatggtaggctgcgttggaagttatttctaagaacagaataaccaaagtctgaatcttttccttactcttgactctaattaaagaaaaattaaatcataatatgcgctgttatctctttcttatagccaagctgtctcagtgacaaacccatacccttgtgaagaatggagaagtgtggaggacttccagggaggaaataaaattgaagttaataaaaatcaatttgctctaattgaaggaaaaaacaaagtgagtttgaagttttaaaatttgaaaatctctctctctttaatggaaggatggtacaataatatgtgaggcatattggagattaataatcaaatagtctggatgattaaatagagcgtattaagtcactttgaaaataccattgacttttagcagtaccattaacttattaatagcttatcagagaaaaataaaaacatctatgacattaaatctatgcatctgtgtagggtgattctgattttataaacatgagaatgaaaaaatgtgtatcatatcatattaaaacacatcattagtttcatggcttccaaagccctttttatataatgtgtgagctccacagcagcataattatacaaattgagtaaatatcccaaacctaaaaaccccaaatccaaaatgctccagattctgaacctttttgagtgccgacatggtgctcaaaggaaacgctcgttggagcattttggattttcagattagggatgctcaactggtaagtatacaatgcaaatattccaaaatccaaaaaaaaaaatccaaaatccaaaccacttttggtcccaagcgttttgagtaagggatactcaacctgcaattgcataaatttgagcgtgtccaaccgctgcagaagtgggaatggcataggcaggttggagtgattgtggagactgctggactgagtgcttgtgcacaaacagccgcgttgtttatggcctgggatttgttttttccccgcacagactgtaagtacccttgttatccaagcggcaaatgtgtcagctttgtacaaatgtgaagcggtcaacaaagtcgggagaggagagagggtgatctccttccacgtgaccagtaagtactcttctctggaggtttgggttggatcactcacacagtgggtactaagctatgtaattccctgttgtttttgccattcatgtgagtggcatggcatttaggaaagaggacttggattgatcattgatgctttcattcataaattacaacttctcaggtatctcctgggcttatgtgaagtcagtgcgtctaactacactggagagagaatggtttcacagatgctttaaaccacaagctctgtgtggtatttacatctcagtcttcagagtctggcacagtgcctggcttattgagcttcagtacatattggtgggcttgctgtggaacagttgatgagggtgggctttatggaggcaatcagaaggacataggagcagtgccctcccaatgctgccgattttgcctgtgcatcttagttttatggataagctttagctgattgtgctgaatggaatattatagccagggctaattcattggcataaatgtagctttcatatcattgagtgttagtgttaatgaagacctaattttaaaattctgttagaattagagattttgctttggatttttaatatattaaacattgcgtagagctcatagtggagatgtggtaaatatctgaggaattcgtttacattttcaagtaatgtgtttggccaaataagatattttgggacctgaattgtctagtttgtttgtcaagttgtagtacatcacctggaacggatagagcttcatttcttttggtactttgtagtagtctgaaagcagcaagatgatagtgagctgtaccaagttaaatcaccattcaataactatggcctcttcattttagggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctaggtagggagacaattctggatcattgtgcagaggcagttggaatgccttaaatgtagtgcaattcaggtgctatgcaaagattactgtcctctaggagattatgttgtaaactggtgcacacttcttcaccgaaagtccttgaggaagaaagaagctaataataatgaaatgatatatcgaaaggagaaaataacaaaacctgatgatggagtaattcactagtatatgcaagggatattagcttgaaccagggaaacttctgccttatcttgggcatccatttatttaaatagacaaatatttgtggaatgcctgctatgagctaggagagtgtcagaaattcacagtggtaaacatgaaggaaaggaggagaacataggcaaccactgggaagtcacagcacagtgaggtctctgtgtccatgagaacaggaattgttctctgttttgctccctgctatagctctagtcatagagcatagcagcatatactaactgctcaataaggcacctgctgcatgaagagtgggatgatgggctgcgtttaagacctagaagactccatgggaaggaagctacattcactgtctgtacctctgggtcatcccacatgatccagcgtagcccaaggtcaatgggacgatcacttcagtgagcagatagctctgtaaattcctccatagaggcactgtctaccccttgtctaacctcatgccttgtgcaaaagctgggcagccatggctttgtctgtgggaaaatcaggcaaatttggggagcgtctctttgtgccacttctctccattttctcctcttgtggtgtccctttccaattcctaggatatatgtgccctctgttttttttttactgttaggaaggaaattgcccaagtaaattcatctataccacagttttagagggtaacgtcttcatcagaggccttggcgtatttgaagaggcaccttctgacagacactagcataaagttcgctagttttaagactcaggtgtcataataagagatactttggggtcaagtcatccccagcatccttcaagtcacaccacatagatcacatggattttctgttggcttgtctggcttcaaggttatggcagaattgagaaagagatgtgaagtaggctcctggcctagctgtgcccagaaaatatgtgctcgcagttagctgctttgcttccctaaggactcctaacttgttttcctaaaacctattcttagaaataggctagaatccagtacatttgcttagacttcaatgtagtacgctgttgaggtaatctcattttgctaagtgttgacgtggattttttcagcatgattccttttgatgttcagttggttgggacaagatatttccacagcactttgatgatctgaagaaagaataaatctaaagtgttcttgtacacttaaacaaatactcatgggcttcattttctttaaatccaagacttcccttagggtattgttgttttgtttgtgttttagtggaaatagcactgaactggtcttttagcctcaccagattctgtaaacagttcaactgtttacttagttgcagggacatggacaagtggtttaatgtcgctgaacatcatttatttcatctgtgagataacgctaacagtcctattctgctcattacataagatcactagtgaggaacacaaattgtgtaaacaagttttataagaattgccaaataaatgtaaggcattattggttgaatgatactaaaatttggcacttccaagagaaatttgaagggattctagggtattattgactagaatcttcatgggagggaagttttcacctggggaggctgtgtctaattagaggaaaaatccataaaggtgaccctgaacctttcttttgtgatgggattaccagctagtatcactaatatgaatgttaaaagccattaatctgtttgcagtgtcctgactgacttgtttcatttaactttacccagtgaccagtgtattttcccagaagttaatatatcaacaagttcctttttactaaatttaaactgtttaaaagtttgctgataccagaaccatttcaaaagttataattccatgttctgtgattttctttttgtgtgtctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggtaaatagaatttggctatcactcttgggttgcagaactttcccagggatgttatctaaaaagccatattatttcttgatgtaatgtagaaaaaaagcagtattggtgtccatgacctggctcatttcacagacttagaattggagtatggggccctgttgaattttcatgaaagccatataggagattagtcagcagtagatcccatgtgactctacagagttagataatagaacaagatgaagggcagcatttatattttctaaatttccctgaaaaacttcacagactacatcatcataaatgagaatgatcgttttcttcctctgttaggcattgtattgaaggatgggaaccggaacctcactatccgcagagtgaggaaggaggacgaaggcctctacacctgccaggcatgcagtgttcttggctgtgcaaaagtggaggcatttttcataatagaaggtcagtgggataaaaaaaaatgtggtacatatacaccatggaatgctatgcagccgtaaaaaggaatctgatcatgtcctttgcagctgcatggatggagctggaagccattatcctcagcaaactaacacaggaacagaaaaccaaacgccacacattctcacttataagtgggagctgaacaatgtgaacacatagacacagggaagggaacaacacacactggggcctactgtgggttggggagaaggagagcatcaggaaaaatagctaatgcatgctgggcttaatacctaggagatggattaataggtgcagcaaatcaccatggcacatgtttacctgtgtaacaaacctgagcattctgcacatgtatcccggaacttaaaagaaaaaaagaaggtcagtgggaagtcatagatacatcctgtggtttttgaagattagtttgtatcttatagacacacattcactttgaatagggcaacgacagatgatttttaatattctttgtactttgtaaattttctcagtgagtatgtattcttttaaccagcaaacataattaatgttgttataattctgcttgcatcacatttcctattcctgcagttcttattgtggaaaaattcttaatcaggcaggatgaatagcctcttctccctgattctgtctttgtttgaatggcttgattaacttatagaaatgatgcctttatatttatttggaaaaacattagaattgctgcctaatcatggcagtcaatgctatccagatagtcacaaggattccgagttttaattggactagagataattaagattcacttgtgaacaatagaccattgctcttctgacatggaaaatttttggtttttatctcaatacgtgtgtatgcagaagtgatgtgaaatctgtcattttcttagctaggaaaagtaatttgtggcagaatattttatcttaagaagtatattcctatggcttttttttttatagcccaccagggaaagaataaaactgtgttgtggggtaaaagaatggtatgcaagggtaagaaagaagtatggtgatagaagggatcgatggatttctatgaactcatcctaacttgtctctcaaagtctagattttggtccctttactctgccaaatctatgatgccaagtattgcatcgagatatgttgacatattttcaaatgtataagcttattagcatttcataaactacacttgcaaataaagatttcaaagaccatggcggttttgtcatttccaaagtgattcatgttttagggcaaatccgcagaatgacgtctagattgtctctgatgctctgcattacctcttgttggtggcctgcagctggttacagatgcctaactaggtaacactggcacagagattatagttacttcttacctggagtgaatgctaagaaaggcagagctagatatttaatactcctgctgggttcccaaatgttatgcgagaatattaatatacaaacacatagaaaacagactctttgaactttttatcctctatgttcaactggacttttaaatctgtgtgtataaatagagaattacttccctaggaccaccagagaaacaaaatttactccaagcataattgtgcttgtctctcaatggttaagttaacttttattttgcaaaccaatttgttacttattttgcaaaccagtttcttacttgtcttcttctctcttgaggccgtagtgggccatccgcacagcttgtggcccggtttgattctccttgcactcttctgatgggaggccccaagtgatgactgcttccttatcatctctttgctaatcactcttagtggaaagcctgtttctgtattttgtttcttccactcagagctgtcctctgaagccctgagcatctgcagctttgcttgctgacttctagtttcctcttctctttcctttcatgagtgatttgaaactcccattaccaggccatgcgtgatgtgctcatcttggctcttcctcttctcctcactcagactcctgccacaagggatggggtagtgtatgtaatggttagttcatgttggacaggcctctttatctcttgactgaaccactgactagctgtgtgccctcagtcaagtagcttaagctctctggtcttctgtttcttcatctgaaaactgagagttgttgaggagattaagtggaatggcatatttaaagtgatgagtgcatagtagatacatggtcattagtaactctcaggtcaaaaaattttgtttatttccctacttggtttcttatgtgatccttttgcaaactctgcacagatcaaaatattgactatcagtttaaaagaagacttttgttttcctcaaatagaaatatttttttttctctgtagagaatgatctgttttctttccatcaaagactgctcttcctctaaacactttctatgtttggcttttaagacattactacttctatgcttaattacttaagaattttattgttgtaagtttacatgagcaatgttttgcaagctttaaattttccattaacaattctgtaggccaggtgtggtggcttatgcctgtaatccctgcactttgggaggccaaggcaggggggatggctagaggccaggagttcgagactagcctgggcaatgtagtgagaccctgtctctacagaaaataaaagaaaaattagctgggcttggtggtatgcacctgtagtcccagctactcgggaggctgaggggggagaatcgcttgagcctaggaattggaggctgcaataagctatgattgtgtcatggtactccagcctggaacatagaaagaaaccctgtctctaaaaataaataaataaataaataaataaataaataaataaataaataaattaaattcaaaaaaagaattctgtagactccattcaagttacgggtgtgtaactgttgtcctctaggatttttccaagttggtaagcttgggattttgctttagtgctaaaatttgtcatcttacaaacaaaaagtataagtttccaactgttgatactcattcaattgtgtctttccaggtgcccaggaaaagacgaacttggaaatcattattctagtaggcacggcggtgattgccatgttcttctggctacttcttgtcatcatcctacggaccgttaagcgggtaaaaaaataatttcccttctgcccatgcacattggttttcatgattaatgaaaactgactggggttctttgagttgtttcttcccattgttattggctcaatgggcacatttttatttcaatacaataacgttcctgcccactttcttttggctggatctcagggatttaattgatagaagccactagagaggaaaagggcttggactgtctagtgtaattaagctttaaaaccttaattctgagctcctttgggggacaagggaaactagaagcagggttataataggaccactctcaaactccatgagttttattggaaaatgagacaggaatgaggctccaataaacagcaataacaagcacacaaaacaacagccaaacaacagtgtgtttatgactggaaggattgatgctttccaggccaatggaggggaactgaagacaggctacttgtccatcgtcatggatccagatgaactcccattggatgaacattgtgaacgactgccttatgatgccagcaaatgggaattccccagagaccggctgaagctaggtgcattttcaattgctattaatttgatattgtgtttaccaggccatctcttcctccattagaatgatgacaaatgtggtgtattcagatgttggattctggtttagaaatattaattccatttcttgaatttgtataatcattcatatagccacttagaggtagggtccctatgtaatcatccaaagcaggacatttggagagtgaagggggagttattaaataattaagccaggacaaaggagtaaactggactatccatgttaaattgggatgtatggtcaccctatctagttgatgtctctgcgtatcactttggttgtatagtaatccaagtctgttttcttgttgctgttgttgttgactctaggtaagcctcttggccgtggtgcctttggccaagtgattgaagcagatgcctttggaattgacaagacagcaacttgcaggacagtagcagtcaaaatgttgaaaggtaaaagcaaaattatgtggtgatctatctttctgttttatctagtctttaaatatgttgcaaggcttgtatcagtagctttgtgcttatgtgggcctactagccacacatgcagtcagcctaaataatgcccttgtgcaaattggaaaaaggatcctcctttgtagctttatgccaggatgcatggtctggcaagcaaagttgggaatggctttcaccttcttgcctggttaccctcgtgcagggctcagccaacacagttgtacttagtggttctgggtacagggaaaaaggactgtggttatattaaaattgtttcttaatatattgtggaatcagataattatagaccatctagagacatggaaaggaagatagtgaaatacaaaaatagcatgttctccagaattggaatatgtaaaagatgttcatatgtaaaagataatttgcaaaacaagaatggttgtgttagaaaaaaatataatgggttatattttttaaattaaaagctttataaataattgttaattctaatagtaacggaattctggtctggccattttcattttaggaggttagacagtaaagcttctttcttcaattgtgatgttctttcattgatgaaggcagtgccaatgaccctttgccaataggttttgtgcatttcaaagctatctttctccatctgccttttttctcttgtggccaagggagtgtgtaattttgaggtggctcatcagagccttagatgtggaccatgcctgtgaattagtgggaagtgtagcagtccatacaggatcaaacacatagtcttagtgccatcagcctcatgtgccaactggtctttccagctggccttaattcgcctgcacagatcggcacagattggctggaacattcggtatagcccctaacacgtgaagatatttaatacatggtgttgcttccttatgaggaagtgctgaaatgatcagaccctcagaatcatagtgaacctgaaatgcaaaaatccagttttgcagaagaagagaatctgggcatgattccactgcagatgtattctccgctttgcaaaaggtttcacaatgggttcctttaaatatcaaactttctggctcacttaaaatatgaattttatttcaaattagaaaatagaatttacacttcacttttgaggaaatgcatgtggtctgtaaactaggtcacagctgtgttaccccggagggtaagttgtatagtggcatgcagggagggagggaccccaattattgaaggaaatgtccatacctatgatttccctctttgtactgtatttgtagaaggagcaacacacagtgagcatcgagctctcatgtctgaactcaagatcctcattcatattggtcaccatctcaatgtggtcaaccttctaggtgcctgtaccaagccaggaggtgagtaactgtgggtggttttggtcacccaattttaacatgcctctctgatagtgtttgagggaaagcagtcaactcctctggccttgattttcttagcttagaatactttgcggattcctaggaataaatatatttcatggaggtttaattggcactagaattaaattattgtaaaactttctctgaattaagaaatgtcatgctactatgatacagtttgttacttgtgtaacagatgtccagagaagagtaaacttccctaaaacttgaaagcttaagggtagttacccccaaaatggaatcatatcaggagattgcactgaaaagcaagtagatgggtgggttttcttctgaaattttggttaatcttgtgaaaatgtgttctggaaaaaagaaaagctacaatataaggggattgggaccagctgatttctacactcctgtcccaatgaaaggttgtagccttcttctaaggtgtttttgggttcatcactatattaaacgcttagtgaggaatatgagtgaaaacccattttccttcctggacatgctgcctgcagggccactcatggtgattgtggaattctgcaaatttggaaacctgtccacttacctgaggagcaagagaaatgaatttgtcccctacaaggtatgtcatctcctaatcctgctctggccatgttataaaatgaagggaaactcaaaatggtacaggttagttttttagttgaaattttgtgaagaacttgtgaggaatcttctcatattacctcttggctgttgtaacttcctcttttaccttctgggggccatatgtttctgttttatgtatgtgattttaatctactgacccattacagagtgtggacatgggggagaaggcaggtatgagcgaggaaaggggagggcagagggtaggacatctctgggttattctgtctctcccctagccatatttggccccgtggagtgtaaatccctctgtgaagagcatcctaatgctgaaagtgtgtctgaatgcaactcaaaatgtggcatttgtcactttaagctaaagaaggagctaggctttgtggaagaaaccctattatgcacaaaacttgccccaagtttcagctcagagattgcataatcctgaaattgatgtcctccttgtctgctttttagtagtttcaattatctccatggtttactacattttaaaggttgtaaacttttaaagactcattttgtattcaaggagtttgtttgttcctttgcttttttatagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaaggtactggctagtgcttcctgcatgctatggcatgctcttgtcagagcagacagggtgatagggtgttacaaggaatttgatcatgggaaaagtccaatactacctcataatttgaaagagacctgaatttctataatagactgcctccattctgtctccccaaaagtgaagtgtggaagccctagactgggaagtgaagcagggctagcctgagaaatctgggtagtccaagtgggctaagcagtcggctacaaccacagcagtgttcttaaaatactggttcagcatttattagtgagagaggccacaagttttctggtagttgactagcctctccattgccttggagagccccagagtggtttgccccacgttgcatgctttacctgtgcaaaagtcttttcattatacctaaccttctcaaaggcagtttaggagccatctgttgtttctaccctaccccaagcggcttatcaagtcttccttccaaccatacttcctcaggcgagtcttgataaatatcctggcctttattaagttatgtttccagtgatattttatttatttgtttttatgtttatttttatttttttgaggtggagtctcatgctgttgcccaggctggagtgcaatggtgcgatctcggctcactgaaaccttcgccttttgggttcaagtgattcttgtgcctcagccttccgagtagctgggattacaggtgccttccaccatgcccagctaattttttttttttttgtatttttagtaaagatggggtttcaccatgttggccaggctggtctcgaactcctgatctcaggtgatccgcctgcctcagcctcccaaagtgctgggattataggcgtaagcctccgtgcctggcctgagtgatattttagtgctctttttgggtggagctgtggtcccagcctaacttccaggacttcagccggctccaggacacactgtatttctgcctccttcagaaggagcagagatagcgttgtggatgtagagatgggtgacaggctggctccccttgaggcataagtctagaagaatagtggaagaaacccactctgtttcccttgacatgaggctacagagagaatttgcatttaactccttttccttagaagctgagaaggtagtgtgaggctgggacttggtctagaagcacatggggaggtggtctaggcttcatttagctgggcccacactgagtggtgctgcctctaccctgctctttgtctttcaaaaaacagtggccagtgagccagaaacctaagagattgagttgttgagaaaaaggctcacagccttttaaatacttacgaatttattactacaactaagtttttgtttactctggtatttgtctccaggaaagaagccataagtcttatctgaccaaagagatgattttgaaacacccatttaatatcttagtgtttatttgtaccagttgcactgaagtaaataccaccaatttacgtaaatttatctttccatgtttctgttatctctcaggaaaaaacaccctcccaggccagatttaatgtatttacagcactttttaagtttgaaaatgaattaaatatatttctagtatttttagttatctattgcagattatagtttgacttttggcctttgtcccaggacaaaacctggagagaagagattcaatgaccctgaatattgttgttttatttttagagttcttgatatgaaactattgtttatccctctgggtacatgacaaaaaacagtgtaagtggcaaatttggaaatgtcctctttatttcccagattatctaggtcagtgttaccttattctacctcctggatttactggttcaatttggctaaaatggaaaaaccagtattgttcctaagggggtatgatgaaggctaatgatactgggattcaggagatttacagaagatagaagcattgactctctgcttctatttcctaaaaacttaactcccaagtcttaaaaagattattactctagcaaacttagaaacatcacactaactcatggaaatactgatctccatcctcctgcctctttggacagctcctgaagatctgtataaggacttcctgaccttggagcatctcatctgttacagcttccaagtggctaagggcatggagttcttggcatcgcgaaaggtaagaaaggttgaggggaaatcagctatcttttcagatcacaggtttggaaataagatgtccagtgtcagccattggtgcttgtttgggattgtaattcattcaccacttctacgtcttttagaagagctctactggggaggctctgtttctgctgagtaagagtggttaaggagttcatgaaattaagctgtataataaaggcttgtcaagcatctactaagtgtgaggcagtcttctgagcactgaggatactgtggtgaacaatcaggcaaagctcttcaccttcatggagtttacagttctagtgggtagagcaaacaataagcaatataaacaagtaaaacgtgttgtaggttagatgagagtaaatgctatggggaaataaagcaagaaagggttatagaatacacaggagcaatgcacttgtgtatgtttatgcttctctgtgtgtgtacatctactttaaacaaggtagacgaggaaggctttactaagaacttgacatttgagcaatgacctggaaaggggaggggctgagccttacagatatcttggcatgagaatcatttttaatttattttacattcatcaacatccatcaaaaagtatttgttaggagtataattagaaacgaggaaggacaggcttcagatgagagcgattaaaagagctaaaattagaaaagtaggccaaacaaaggctgagatggggacgtgacaagttacaactattccaaaggttgtaaacaccaagcggggagcaaggctggtggcagtgattcccctggaaaggataaaaggtgtaattttatattaggtaacaatacttcaaattaaggatcaggaagaactatcagttgacagaatgtattcatgcagcttaatgaagaaagaaagacttaagtcatatttttttttgtttttcctaaattagaatgaaatcttcaacccatgttttccccttctcatagcattaaaggcctcaggctctttgatgtttctgctaggtagctcttatgttctctctcccaaggggaaggaggagaactgggaccttatagggttttcccaaagagaaaggccctttacacttcttggagattatgacttattattaccatttttttatggccggaattcgccacttagtcagggttccttttggggactaggaagagaatggaaatgaatgtgggaatgctttaactttccttacatctaccagactatttcttgaatccacttggttgtcgggttaaaaaaggaaactttttgtttggggggaaaagtcaaaaacactgtctgttttttggaattgccagtgttgctcaattgtgctagataatgtgcttctgaatatgccttgttcagaggagagtgccatacagatttgaggtgtgggaaggtcagcaatgcctggcttacatgatcacttctccaatgatttaagaattctccttttggccaggtgtgttggctcatgcctgtaattccagcactttgggaggccaaggtgtgtggatcacctgaggtcaggagtttgagaccagcctggccaccatggtgaaaccccgtctctactaaaaatataataattagctgggcgtggtggcacacctgtggtcccaactacttgggaggcagaggcaggagaatcacttgaacctgggaggtgaaggttgcagtgaactgagattgcaccactgcactccagcctgggcgagagtgagattccttctcaaaaaaaaaaaaaaaaaaaaaaaaagttttcttctaagccattgattcatttcttgtgctccccaagactcattttcttacaaaatatcatgtggagctaaagctgccgagtagtaggaagttagctgaagtttggaggatacagagaaaggagaaactgagaagctaaaaggaagagaaagaagtcaagatgaatctcattgtactattaatgcactagaaaatcaacctgacttgtgataggctgaaattgccttaatagacctttataataacccagcactttgaaatcaggggaagccacattgggaattgtttatcagagccagtctggcttcagcttcatacggaagggggaaaccaacaaagagcactaaaccaatgagagccccttgtttctgatttccgtgcattcattcaaaaaacaaatcccgttctcggacctccttagaataacacgttttaaaccaaatatggggccaggtaaaaggaatgtgtggatgtgaccagaaacacactcttttgtgtcctagaggagcctatttatgattccatcatcatattataacttaattatttaactccaaaggctggggctgtttatggaataagcagatgtgtgtctcagcaaagctcacagacttttttcctgaagtgttgataaaagatactaacccagtccttgttaatcagttggctttctgatgtgggatttttttttgatgcatgaggtcacaacagatgtgaaagagatcagctgtgccgagacctaatgcacacatgattctctttgcagtgtatccacagggacctggcggcacgaaatatcctcttatcggagaagaacgtggttaaaatctgtgactttggcttggcccgggatatttataaagatccagattatgtcagaaaaggagatgtaagtttcaaatatgaacccagtgcttggttaagtaacagaattaaaactcctcgtagagagcttcaggacctgtgttcaggaacagaggaagtttttttcttcagatatttgctaatttgggttctgaatccttgtcttctacccctgtaggctcgcctccctttgaaatggatggccccagaaacaatttttgacagagtgtacacaatccagagtgacgtctggtcttttggtgttttgctgtgggaaatattttccttaggtaagtcatttctttttgtccttccatccagactccaaagaggaagacaaaagttgtcttttcctctcctgtacttcatgtctatcaggcaaaacttctcggaagctttgaaaaaaaaaatagatacataggtgatgaggatgtgcaagattcaggctcagggttttctataagagaaaatcaaatcaaagaatgtctcctccctgttttattctaggtgcttctccatatcctggggtaaagattgatgaagaattttgtaggcgattgaaagaaggaactagaatgagggcccctgattatactacaccagaaatgtaagactttaagaagtattcctgtgttctctttctttgctcgcaaattctccttgcctggaagactttccattatatagaccttcttcattgcccagttagtgtcctgcttttactttggggcctttcttgataatttcaagcatggagtcatcacttcttgaaaagatagtactttattattcaaagcaaccagttagtttttattagatgttgctttaaatgttttctatacacattgagcctctggagtatgggactctgtgtcttacacagttttgtatccttatttagcatctcacctcgtcagctctttacaaatgtgtactcatttaagtgcttattttcagcattcaggaagaaagaggcatttaatgaaatcagtgttttgcttctctaggtaccagaccatgctggactgctggcacggggagcccagtcagagacccacgttttcagagttggtggaacatttgggaaatctcttgcaagctaatgctcagcaggtttgtcacctccatccaagaagcacctacaaagagtacttagatgtcaaggactttcctactgcctgaactgtctcatggctaccatgccatcctctcagccattgaataatctactgtattcttctacatctgagtaataatgcttttctaaaagctgtaattacccttttagacagataggattctaatttataacccgggagcagaccactctgatttctacctacttatctttttgttatattttcaaatcctcttctaaagttaaaacaaagaaaaaatctggttgatccacagaagatcaacaatggaagaaatttcaagaaatttttaataaattctgcaggcaaaaatacatctaagctatgcaaaagagatggtttctgtcttggtatcatcccaggttcttataacttccactggaagattttagagttgtagtgtttactattagaatgttatttaatctctagtcaatgcctcttactacaatggaagtgaatttcctctttcttttcttttgaacagctgggggacgataggtcagctctatttttatcaataaaccttccaaacatttacagatatcaaatagccctttatttctttttcttgatgcaataatattaagttgtgcaaccttttctcaaaagacccattttcctacccatttgttgcttttctttagactgtcatcagtttttccattgccttgaaatgtggtggctaaaactggatgccatgccctttgaagggcttggctcgtgtggttagggctttgtgaatgagtgattttttgttctatgtagctccttgtgttctgttgttacctctctgaccacagcctgctttctcttcattgtaactgcacttccctgtgggctgcttacccatcttgtttttagttctctcctttaatataccttccatttcaacagctttttgtttctgacacatgatttgtattgttgtcttaaagttctatgttcagatatgaaagccacacaccctatgtagccaagaagtccctgtgccctttgtttttaatgaaaaggcacttgaagaactgaagccataacaacagtcttctgtgtttattgtttcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcaggtactgtatatggcctaacatcccccgggggagggtgacttcaaggccatctcgggagggggattggaagtggaaggaagaccttgtctaaggctgttgcatcccacttccacataaccttagccctgaggttaacataatggggaatgctcctggaagagggcctgggtaggtgtgcttcctcccatctgtagcccacgctgctgccacagcattgcctttaagaattccaagccctgcagctgcaatagctggaatgccacagtttgctaatttccagaataaagagacgagttttacaaagacatctgcatttaaattatccccgtgtatgcttttattaatgtgaattaaatggcttaggagagattcagaaaggaagagttctgtgcttgcatgagaacatgcttatggctctctggcaaggatacagaaagccatgggtctgtgtccggaattagactggacactgcatctcagaagcccctcccacgtctgattttcagcattttatttgcataatgggatgtctgggcttatttaaaacacatgcactgcagtcctttcctgatttgcagaggggttctaaaggcagctttcttttttctctctcccagcacctgtgcataaggaaagagttggtgtggttttctacaatatgatattaaaattgccctttactaaggctgggactacttcattttgctttgtttctttcctaacccgtttgggtgttttcctgctttaatggaacccctgacagcatgggtccagcctgccagcccgagtgtgcctgggctgcagggaggggcagggagctctctcatgtccagaacttggccaggttgccacatggcaggggatgctaaggagaaactcgtggacagtttgccctctagagtcgtgtggggcagcagaaacactgatgggaaggaagaaagcttagaagccagcaagacagctgaccgttccattgaagtcaaaagcattaggcatatttttaaagaactttgccgtatattatcagatgttgcccacatcatgacactcagagtcaggcaaggtagaaacaatgatctttttttttgatgtattattgaacatgaggctcagttctattacctgagggcagtacaaacttgtagttaaagatcaggtattagagtcagatagaaatgagtaggacccccaagtctgtcttgtagcagctgtgcaacttggggcaaatcatctaccctctgcctcagtttctttatctgtgaaatgagacaaggtcagtggtgctgtttgaaaatggctgttttgagagttataagatataatctatttctaagcacctggcccttgaaagcactcagtaaaagatacctattaagtgagctgcttaaaatcacatccttgagatgaatccagttcctctgacccctaagtccatgttgtttcctcccatgccaaggagggccctcagagagaaacagtaatgagatgagactacaattccactcctgtgtttacacatttccagttcaagttgagctggccttttagtgtgacagttgttcccacacaccattattgcctccccctttatcagaaagccatttgatcatgaactacattccatgtgttttctgtgaccaagtagagtgatgatccgagtcggcagcctcctggctcaccgggtgctttgcatatggtgctgagcaggagaagaaatcatgtttgtgtaatggaagcaccaaatacgatgttggatatatagaagggctgctaacgtttatccccagaagcgtggacaaatgtgacaccacactcccagcacaggcctggctcctattttctgtctgtgatttttgaattggtttttccagcccagtttctcttttatccagccataatttgaaaaataaaatggaaattggaatcttttgtctgcatctcctctccacctcctccaccttttttcctttctataaaataaaactcacggtcacattttaatcatctggttttgaagaaaagcagatagaggcatttgcacacggcatgcttcattctgttgctctcctggggttctgtttctctggggagaatgagttgaggctggggtacttctcagggagcttgttctatcctcttacgcatttctggccaagtacaaaagctgagcagtctttctccttctaattttcaattctattgcattataaatagagttggacagagatatcactgtgggagctagcttcatgatttgttgcccctttaaaccatttgaaaaatatttacttagcatttatttagagaaaaggctgagaagtgtgtgggggagggaccactcatgtctagacttagctttgcctctaatttcccctgtggaccagctctggcctcaagtttgcatgcttcctgcaagaaaacacatacttgctgggctcatctttctttgagggcagtttggggaccatcggcaattgctctgtcattttccctgggagtttcacctcacacatcaagcagcttatcaaaaatttctttgcagttctctcttagagaaaggttttggtacataccattttcttcattttgtaattgttagggatgattaaatggcccttgtagattgatgcttggggcagcctgctagctaggtattcctgagtttggctctaccattagactgtttgcagtgggactgtcctttctgcactttttgtctgtttcataccccgtacttacacccctgaccctgctactgcatgatcagtgcatgcatgacaagagaacagtgctgtgcacatactgggtgcttaataatggcttgaacaattgtgtctgctgttttcttctttcttttccctcctgatactcttccaagggagtctgtatggagtagagtaaaacaaaacaaaaacttcacatgggctttagtgtctgaaggcctaagtttgagtcccagttctaccttttattagccattttctccctaatccttgactccctcatctccaaaggggaaatagttaaaagacctgtttctccgtcttaggagaaacagatgcaccattgtctgtgaaaatgctttgtcaatcatgagaggatcatgccatttaaaaaattactggattaagaatttaaggagctgtcctttctaaggcagctgaattattgtccaaactcgccaaccctagttgattctatcccctagatatctctagaatgagcccatgtctccaaacctcatgggcattccctttttctagccaagctgcctttctttctcctgaagaagtgcagtatttgtctcttgggtcttatgcctctagtcttattcttttcaatccagagtcaattctctaaagggcatatctgatcttgtcaatcccatgcctaaaatccttcagtggctcttcattgccctcaaaataataatccaaacattccagttatgtgattttggataagttcctcaaattttctatgccttggtttcctcatctgaagagttgggatagtaatactcacccctagagaggtaccgtggtgaacacatcatgagatgctgcttagacagcttctggcacagtgtcaggcttgcggcagattatcagtgagggcttcctgaacaagtgaatgcaggaatgattgactacggtaccagtagtgtttgacaactgttacttttaggggttggacttagaaagtaggctttgcttgcaccctgtgtatcatatcctcttaacttgtggagtttcctgagtgaggatgtcaccggaaaatctcattctctcctctctctatagggaggaaccagcctcttggggtaggggagagagaattaatttccattcttctcctttggcccaaggtctatgcagcatgttccagaagtctgcttgtagtgggaagtaggctggtataggaatgaagaatgtattttctgtctcggtgggcccttccagtgaataggacttcccttccctccacttgggctgtaagtgattttgatagcatcaactagactcacccaaagccacacggccgggaaggagcattctcaagaaggagaggatctgttgttcaacaagtcttattctttggactcctgaaggaagctttggaagtcaaaggagaaaaatgagctttgtttgaagagggcattattcttcctaagagcaataagcccaacattctctatgtcattcatcttcccaacatccctgtgagctggggagggagtgctactgccaacacatcttatagatgggacaagagggtcacagaaatattcatgactttctcaagtttctgcagtcagtggtagactctgaaataggcaaaatatcttgttattctcaaaccactgctctttcctgagacagcaactctgggggcgaaaacgaggggacagtgagactcagcccaccttctctttgcacaccaagcctctgttacatggaggaggaagaggttgtcttcaaatcactgctgggttcagtatcctttaaggagaccttcagatgtttcctctgcctatctttcattgaatggttgctctgtgagcattatccagaaaaactttcccaggagatggccagacagatgtgaaacactcagtaatatatccagagctcgatggaggaatcccatgcaatcaggaagccaagtagaaggcagttgatcactccatctgctgttgttgtctttagtccagaactggacctcagaagtaggattcaaaagaacaggctcatcgagactcctcagttatattatacttttaaatgtactttctcaggaaattaagccttccatgtgtgctagcagagaaagatttttattttgttttgtttttctaaaggatgttttgaaggttgctattaagtttgtggttgaaagataatgaacttaggtagccgatctgcagtcaaatataccaccactaaaatataaatatttgttcttttgcagtcagtatctgcagaacagtaagcgaaagagccggcctgtgagtgtaaaaacatttgaagatatcccgttagaagaaccagaagtaaaagtaatcccagatgtaagtacgtcttttaaaaatagtcttagaaataatacaaaggatgaaacactagctagataaatattagcctaagcattaaagttttggagcctcattagaaggctgccctcgagtgtgtgtatcatggggtcattatggagatggaactttgtttttttcataagtaaagcccttggtccaaggttcaagacagtgtagctttctgaccaatttcactaaagtgcaagtagtgtcatagtgaagacagcgatggtaacaggcattctcagctgctgatttgtaaattttctcttctccctggcctgtgtctactcataggaagcagttgcttccttttgtagcttggacaatttgtggctatgatacctttatgttcttccacaggaccttatttgatagacatgatagatgggttgagaaatcagcttaattaaatagttggtcattttatatgctcaattaactgtgccatctcattgtctcttaaaaaggacaaccagacggacagtggtatggttcttgcctcagaagagctgaaaactttggaagacagaaccaaattatctccatcttttgggtaagactcagccatattaaaaagacaaatttcaataggaatttttggaaggaacttaggactttcagtgtaagtgcagaattttccctatggggtctttgttggttggagaaattagcatcaatttaacaaataaagaatggaaactaaccacacaataaaattaagtgataaatctaaaaataatctgaaataaattagagaatttggtcaatttttatgagaattcatgaatactagggaatttctgtgtatatttactgtggtcagtaatggctaaatgaaaaaggtgattggatgtgatccgtaaagctgtcaatatgattacaatctttgtggactctgaagaatttttaagtctgtatacaaatgggtgcatctgtgcttaagaagtatgatatataaataagccaatatctatttgtttgagacatttaaatattattgtctgaattcgaagtatttcattgtgagaaaagtattaaaattagttttaaatataatctcccttctatggctcagtaggaatttgtaggtgtcttgaatacgtgtacgttctcttaacataacaaatcaatgaaaatctatatttataagaataatagaataagtgtagttatgtatttgctggagtttatttgctagagtattcttacctaaaggtaagaatagaggaggttttgatctgcttataatcttttatataaaatgggaatactcatgggtttttgaataatgctcataccaaaaagaaaacaaacaaaaaaaaccccaacatattaaaaggtgccattgtgctattttattgttttctttaaggcccaaggtaagaaattgtgaaagtcaatgatatgtttcattcattgattcaaaaaatgtttattcggcaagtatcatgtgcagagcaccatgccattgcttgagacacctacattagttttgttggggttgaattgaaagaaaaaattgtatttctcattatttgaagtaacttttaaactatgtataaacacgagttactaaaattcccttttgcagttttaacatgaagaagttggggaaaacacctattaccgggaaaaaacaccttagaatggcttgtgaaagtgtaaatcctgaagttttagatcaacacagcctgcatttctaggctttgacatgattaccgtctgtcaggattccatgccattgaaaacattttctagttgctgctgagtgacaggggttctcagtccttccaaggaatgtggttttgatgagtaaaaagcagcgtttgatatgtctggcttgactgcacacatgcttcaagttattaaagtttaaagttgctcaagagctttattacaaccatacacatgccccgtaattcccaaattgccacaataggaaaagcacaagtgaaatttaagaacatcccaatttccttgaatatcatgcaagtggccctttggcgcctgtcactgtatacaaatttgtcaatctgcgaggccataaacatgttccatcagttggggcctttgcataactcgagagaactgcctttcatctcatttgaggcttgaaagacttggacctgagtaagaggacttatctgcaactactaattcatgcgagtacctgaaaatagaccttgtccctgtaaacctgctatgctgattaacaactgggagagatacggggctgcggtctccagggagatggcagccatatggagttgggaatggggtgagggtaaaaagcaaaagaattgtcttctctctgccaactcctttgtttgccatttcttctgcagtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 2 - DNA, 6055 bp, Homo sapiens
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 3 - DNA, 16279 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaaggtaagcggtcgtgccctgctggcgccgcgggccgctgcgagcgcctctcccggctggggacgtgcgtgcgagcgcgcgcgtgggggctccgtgccccacgcgggtccatgggcaccaggcgtgcggcgtccccctctgtcgtcttaggtgcagggggagggggcgcgcgcgctaggtgggagggtacccggagagaggctcaccgcccacgcgggccctgcccacccaccggagtcaccgcacgtacgatctgggccgaccagccgagggcgggagccggaggaggaggccgagggggctgggcttgcgttgccgctgccggctgaagtttgctcccggccgctggtcccggacgaactggaagtctgagcagcgggggcgggagccagagaccagtgggcagggggtgctcggaccttggaccgcgggagggcagagagcgtggagggggcagggcgcaggagggagagggggcttgctgtcactgccactcggtctcttcagccctcgccgcgagtttgggaaaagttttggggtggattgctgcggggaccccccctccctgctgggccacctgcgccgcgccaaccccgcccgtccccgctcgcgtcccgctcggtgcccgccctcccccgcccggccgggtgcgcgcggcgcggagccgattacatcagcccgggcctggccggccgcgtgttcccggagcctcggctgcccgaatggggagcccagagtggcgagcggcacccctccccccgccagccctccgcgggaaggtgacctctcgaggtagccccagcccggggatccagagaaccatccctaccccttcctactgtctccagaccctacctctgcccagtgctaggaggaatttcctgacgccccttctcttcacccatttcctttttagcctggagagaagcccctgtcaccccgcttattttcatttctctctgcggagaagatccatctaacccctttctggccccagagtccagggaaaggatgatcactgtcagaagtcgtggcgcgggagcccactgggcgctttgtcacattccaccgaaagtcccgacttggtgacagtgtgcttcccttccctcgccaacagttccgagtgagctgtgctttagctctcgtgggggtgggtcaagggaggatttgaagagtcattgccccactttacccttttggagaaatggcttgaaatttgctgtgacacgggcagcatgggaatagtccttcctgaaccctggaaaggagctcctgccagccttgcacacactttgtcctggtgaaaggcagccctggagcaggtgtttttttggaactccaaacctgcccacccaacttgcttctgaaagggactctaaagggtccctttccgctcctctctgacgccttccctcagccagaattcccttggagaggaggcaagaggaaagccatggacaggggtcgctgctaacaccgcaagttcctcagaccctggcacaaaggccttggctacaggcctccaagtagggaggagggggaggagtggctgcctggccacagtgtgaccttcagaggcccccagagaaggacacctggcccctgcctgcctagaaccgcccctcctgtgctccctggccttggaaggggtatgaaatttccgtcccctttcctccttggggcccaggaggagtggagggtcccgggagaatattgtcagggggaaggcagggggtgtcatgggaatgggtgagggggctgaggtgcagaatccagggggtccctgcaggagccgcagtggtaagctgtccagctggaagcctggtaactgttgttttctcttgagaggggcttcctgtgaccttggctgtctctgggagcagggctggggtacctgagtggggtgcatttggggtgtgtgggaaggagagggaaagaaagatggacagtgggactctcccctagcagggtctggtgttccgtaggctagagtgcccctctgctctgcgagtgctgggcgggaggggagttggtgagagctggagacccccaggaagggctggcagaagcctttccttttgggtgctgtcaggtccgcatgtcttggcgtgttgaccttcacagcttctggcgaggggaggaatgatctgatgcgggtggggagggttagaggaggcctcaggcctaaggtggtgcagggggccccctaggggctgggcagtgccaaggcataaaagccttccctggtccctggtggcatttgaaggtgcccaggtgagaggggcttggcacctcctcaccctgggagggagaagaaaccagggaacaggtaggagtgggagacaggtgaggctttggaaatctattgaggctctggagagatttgtgtagagaggaaaatgtggttctcccccagggtctcctcctgggtttttaccctctaagcaacctgtgggcatgctgggttattcctaaggactagaagagcttggatgggggagggtggttggtgcccttcggtcctcggcacccccctccgtctccaacaccagctcaccctggtatttgtcatgtcagcaggagaaggtcaccatgttgtttttctcgcccctagtccttccttcctgccccagtccaaatttgtcctcctatttgaccttaatacttaccatggctttggaccagggaactagggggatagtgagagcagggagagggaagtgtggggaaggtacaggggacctcgacagtgaagcattctggggttttcctcctgcatttcgagctccccagcccccaacatctggttagtctttaacttcctcgggttcataaccatagcagtccaggagtggtgggcatattctgtgcccgtggggacccccggttgtgtcctgttcgactcagaagacttggagaagccagaggctgttggtgggagggaagtgaggagggaggaggggctgggtggctgggcctgtgcaccccagcccctgcccatgcccatgccttgctctctttctgtcctcagtggtcccaggctgcacccatggcagaaggaggagggcagaatcatcacgaaggtgagtccccctggctgttggatggggttccctgtcctctcaggggatgggtggatggcctaattcctttttcttcagaactgtggggaggaaggggaaggggcacaggaatataaggatcaagaaagaaagagctgggcaccacgaggttcaccctcagtttcgtgaggactctccgctgttcaggtctctgctagaagtaggacttgttgcctttttcttctgctctttccagtaaaattttatttggagaaggagtcgtgcgcacagagcaggaagacagtgttcagggatcctaggtgttgggggaagtgtcccttgtttcccctagctcccaggggagagtggacatttagtgtcatttcctatatagacatgtcccatttgtgggaactgtgacccttcctgtgtgagctggaggcacagagggctcagcctaatgggatctctcctcccttccctggtttgcattcctttgggggtggagaaaaccccatttgactatgttcgggtgctgtgaacttccctcccaggccagcagagggctggctgtagctcccaggcgccccgcccccctgcccaaccccgagtccgcctgccttttgttccgttgtggtttggatcctcccatttctctggggacaccctggctctccccaccactgactgtggcctgtgctctccacctctggggagggaaggccctggggtcttccttcccgcgagtttccctgacctaaatctggcgtggctgggtagtggccagcagtggtgatgcccagcctgttctgcctcctccttccccaccccaggagccctttccttggcctaggacctggcttctcagccactgaccggccccctgcttccagtgcgccacttaccccttccagcttcccagtggtctctggtctgggagaggcaggacaaaggtctttgtttgctggagaaaaggttgtctgcgataaataaggaaaaccacgaaagcctggttgttggagtgtacgtgtgtgctcccccaggcagtggaggccagccctccttggaggggcggctgcctgatgaaggatgcgggtgaggttccccgcctccacctcccatgggacttggggattcattccaaggggaagctttttgggggaattcctaccccaggtctttttaccctcagttaccaaccccttgcccaggccagaccttcctgctatcccctcctgggccacaagcctggccctcctctgtcccaattgtgatgaaggggcagttcaaaacttcttgattagtcatcttctcccctatcgacttggctttaaaaaatgaccttttcagacttctagtctcgttcactctttttgatgatgctttgccgtaacccttcgtgggtagagaaggattctgtgcccattggtggtctggataaaagaaatagagacctcacaggaagcagtggactggcctgtttccccactgttctttctgttttcacacctgtggccttctccccaccttcttcccaatcaacctattgtgtacatagcccccctcattgtcctttattcttctggaaagcagaccttggagggaggagtgagggggaggctcagctgtggtctctggggggtgggggttgggagctggggtggaagtccacgaagcatacacttaagatgctttggtgaagttctaaacttcatattacccaggctgaaaaaagagcacttgttcctagggctggaaatggaagccaaaacaccacctttttcagcctgtttcagcatctttagagatcagcccaacccacttacacagttgagcagagttggaggcctagagaggggagggactggcccaaggtcataccaactcatggccagagcctgggcctcctcactggccaggtgttatttcttccctctgggtagggaacctatttcagggacaggattgctatgtggtagtggtggtggggtgcgataggcgtggcaggctgggccacaatttggagtagtcatgccagagtcctgcatttatttattctcaagggccccgcctctgtggcccagaattaccccttcatgctccagtgcaccccaggcttcgtggccagcctgggaaactgtctctaccctggtctcccttcagatcagcttctagaaatgtttcgtggctacagtggcagcactgttttttccatgatgcaagcagtttgccctcttgggcggggttatcagtggctggcagggctggcacagcgtgtccgcccactgccacctgtgggttccaggagggcccagcccctgtgctgatgcccaccaccttctcagctcatgtctggggaagaggactggcagggggaaaggtgcctcctcctgaaaggtgcctcctctgtttttgcctaatataggcttgggaacactttgatgtcagctaattctgactcctttacttactagctgtgcggccttggggcaacttacttagcctctttgagcctcctgttccccatctgtaaaatggaatctcaatagtgtctaatagtaccatgtggagaaacttgtgtgaaatgatagctgtggactactgtacacagtactcaggatgtagtaagtgctcaataaacagctgttggtatggttgacgttatggtagtggttgtggggaggacgtaggaaactggagactagcttggcaaagctggctcttcctccttttagggaaagcttagagcatccccatggggtatacccatactcagactgtcctctggcatcgaggttggcccaggattcagttcagctgtcacagtgaggtggcgggatcagatgtggcaggccatgtcccttggaacttgagtacatcgtgtgatctctggaatgaaaacaggccttcaccagtgttgatggtggaaagcttagggaagtgcttcaaacacagtaggagggacttacgttagattttggaaggacttgcctgattcggaagctccaaagagtggcattacagagctgggtggagagaggggctagccatcttttgtgtcgcccaccgggctcatgtgtcatcgcctctcatgcagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcaggtgggcatctttgggaagtggggcaagggggggatagggaggggggtaacactttgggaacaggtggtcccaggtcgtttcctggctagatttgccttgtctggctcctgcccctgagttgcacaggggaggtatggtggggtcttgccttctgtggagaagatgcttcattcccagcccaggttcccagcaagccccaaccatctccttctccctgatggttgcccatgggctcaggaggggacagatggatgcctgtgtcaggagcccctctctccctctcttggagagagtcctgagtgcccccccttcttgggggctttgtttgggaagctggatgagcctggtccatggagagtttaaaaagtcttttggtgttacctggtaatggggcacatctcagcccagatagggtgggagggagctgtgaaacacagggagggggttgctttcgggtatctactaggagtcagggtgaagcctagagaggatgaaagaaggggaggggatggggagtggtaagaacctaggatttgaattcccagcctggccaacccttgcagccatgtcttggcctcaagtggaacaagggctccttgaggccagcagggttgggggagttggggtgggcctgagcctctttcctgctagagctcttggtcctccctgcctccaccacccatccctgctctgcagaacccctgggtgctgagtggcaggagccccagggttgtcccatctgggtatggctggctgggtcactaacctctgtgatctgcttccttcctttccagattatgcggatcaaacctcaccaaggccagcacataggagagatgagcttcctacagcacaacaaatgtgaatgcaggtgaggatgtagtcacggattcattatcagcaagtggctgcagggtgcctgatctgtgccagggttaagcatgctgtactttttggcccccgtccagcttcccgctatgtgacctttggcattttacttcaatgtgcctcagtttctacatctgtaaaatgggcacaatagtagtatacttcatagcattgttataatgattaaacaagttatatatgaaaagattaaaacagtgttgctccataataaatgctgtttttactgtgattattattgttgttatccctatcattatcatcaccatcttaacccttccctgttttgctcttttctctctccctacccattgcagaccaaagaaagatagagcaagacaagaaaagtaagtggccctgactttagcacttctccctctccatggccggttgtcttggtttggggctcttggctacctctgttgggggctcccatagcctccctgggtcagggacttggtcttgtgggggacttgtggtggcagcaacaatgggatggagccaactccaggatgatggctctagggctagtgagaaaacatagccaggagcctggcacttcctttggaagggacaatgccttctgggtctccagatcattcctgaccaggacttgctgtttcggtgtgtcagggggcactgtggacactggctcactggcttgctctaggacacccacagtggggagagggagtgggtggcagagaggccagcttttgtgtgtcagaggaaatggcctcttttggtggctgctgtgacggtgcagttggatgcgaggccggctggagggtggtttctcagtgcatgccctcctgtaggcggcaggcggcagacacacagccctcttggccagggagaaaaagttgaatgttggtcattttcagaggcttgtgagtgctccgtgttaaggggcaggtaggatggggtgggggacaaggtctggcggcagtaacccttcaagacagggtgggcggctggcatcagcaagagcttgcagggaaagagagactgagagagagcacctgtgccctgccctttcccccacaccatcttgtctgcctccagtgctgtgcggacattgaagcccccaccaggcctcaaccccttgcctcttccctcagctcccagcttccagagcgaggggatgcggaaaccttccttccaccctttggtgctttctcctaagggggacagacttgccctctctggtcccttctccccctcctttcttccctgtgacagacatcctgaggtgtgttctcttgggcttggcaggcatggagagctctggttctcttgaaggggacaggctacagcctgccccccttcctgtttccccaaatgactgctctgccatggggagagtagggggctcgcctgggctcggaagagtgtctggtgagatggtgtagcaggctttgacaggctggggagagaactccctgccaagtaccgcccaagcctctcctccccagacctccttaactcccaccccatcctgctgcctgcccagggctccaggacacccagccctgcctcccagtccaggtcgtgctgagcaggctggtgttgctcttggttccgtgccagctcccaaggtagccgcttcccccacaccgggattcccagaggttctgtcgcagttgcaaatgaaggcacaaggcctgatacacagccctccctcccactcctgctccccatccaggcaggtctctgaccttctccccaaagtctggcctaccttttatcacccccggaccttcagggtcagacttggacagggctgctgggcaaagagccttccctcaggctttgccccctgccggggactgggagccactgtgagtgtggagacctttgggtcctgtgccctccacccagtctcggcttcccaccaaagccttgtcaggggctgggtttgccatcccatggtgggcagcgtgaggagaagaaagagccatcgagtgcttgctgcccagacacgcctgtgtgcgcccgcgcatgcctccccagagaccacctgcctcctgacacttcctccgggaagcggccctgtgtggctttgctttggtcgttcccccatccctgcccaccttaccacttcttttactccccccaccgcccccgctctctctctgtctctgtttttttattttccagaaaatcagttcgaggaaagggaaaggggcaaaaacgaaagcgcaagaaatcccggtataagtcctggagcgtgtacgttggtgcccgctgctgtctaatgccctggagcctccctggcccccagtacaacctccgcctgccattccctgtaaccctgcctccctcccctggtccttccctggctctcatcctcctggcccgtgtctctctctcactctctcactccactaattggcaccaacgggtagatttggtggtggcattgctggtccagggttggggtgaatgggggtgccgacttggcctggaggattaagggaggggaccctggcttggctgggcaccgattttctctcacccactgggcactggtggcgggcccatgttggcacaggtgcctgctcacccaactggtttccattgctctaggcttctgcactcgtctggaagctgagggtggtggggagggcagacatggcccaagaagggctgtgaatgactggaggcagcttgctgaatgactccttggctgaaggaggagcttgggtgggatcagacaccatgtggcggcctcccttcatctggtggaagtgccctggctcctcacggaggtggggcctctggaggggagccccctattccggcccaacccatggcacccacagaggcctccttgcagggcagcctcttcctctgggtcggaggctgtggtgggccctgccctgggccctctggccaccagcggcctggcctggggacaccgcctccgggcttagcctcccatcacaccctactttagcccaccttggtggaagggcctggacatgagccttgcacggggagaaggtggcccctgattgccatccccagcaggtgaagagtcaaggcgtgctccgatgggggcaacagcagttgggtccctgtggcctgagactcacccttgtctcccagagacacagcattgccccttatggcagcctctccctgcactctctgcccgtctgtgcccgcctcttcctgcggcaggtgtcctagccagtgctgcctctttccgccgctctctctgtcttttgctgtagcgctcggatccttccagggcctgggggctgaccggctgggtgggggtgcagctgcggacatgttagggggtgttgcatggtgattttttttctctctctctgctgatgctctagcttagatgtctttccttttgcctttttgcagtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcaggttggttcccagagggcaagcaagtcagagaggggcatcacacagagatggggagagagagagagaaagagagtgagcgagcgagcgagcgggagagcgcctgagaggggccagctgcttgctcagtttctagctgcctgcctggtgactgctgccttctctgcttttaaggcccctgtggtgggctgcaggcactggtccagcctggcggggcctgttccgaggttgccctggttgcctgagtggtaggctggtgtggcttagtgtagtggtgtggacgcaagctgtgtgttgtgtcctgtggtccttctgctcatagtggctgttggtcctgatgttattactacctctggtagtaatgctgagaagctgaaagccgattccaggtgtggacaatgtcaacaaagcacagatgctctcgctggggccttgcctcggccctttgaagtctgcatggctgggcttctcactcactcagtgtttcttgctgggggaaggaattgagtctcccacttcagactgggcctccctgaggaaagggttgtgtctccccactcagactgaggttccctgagggtagggctgtgtctctcccctccgacctgggctccctgatagggctgtctccccgctcagactgaggctccctcaggccagggctatgtctccctcctcagactggggctctgagggcaaggggtctggctgttcgtttaggatggggcacttttgcctacacactgaaggagctgtagcatccaagaatactagatacctttaatcctccaccagtcatggtgacaaccccaagcagcccacacattttcaagtgcccccaggatgcgtggagggaggggtctgtgcccattctcctgacattagcctgtgagctccgtaagcccgggcctcgtttacgtacctttgtgagccccgggcatctgtacctctttcctttgcccatactggggaccaaggaagtgtcaagtgcatgagtgaatgtgtgactcagttcagagggtgaggtcaggagcacagggtcgggacaggtggctggcatcttttaatgccttagcttatgttctttataccaacttggcctgtgctcagagtgagggaggccctgggggtcagggtaagcgtcagtcagggaggcaagactttgtggggatttcctagacagggccaaggcacccccagctcaccccgaggctgtgttagggaagtccttggagtgtctcccctcccccagcaatgttcttgtggcttgtgtgtgctcaggggatgctgggaaccaggcctgggtagttggtgtggggtgctgtctgtcttggccctatgtgaaaccaagagggcgtatattagtgctggggtgggggctctgcctaacttcagggctggatgaggggagtctcagttccccaggggtccttgggaaagataagggacttgacattttagggtttttaggtgattattctgctgatgggggtttgtgtgaagtgacctgggagctaactgaagttactctaacctcccaatacctttacccaacccccaagctggctgtatctgggaatatcagtttccaaaattggaggcttaggactccgtttcggggctccccagaagggtagggcctgttctgcctccttctcacaatcacccaggggcaggggcatgctgagaaagttcttggaggccccctttgcttcagctggagtagtgaagccgccgaattgtctctccccatcctaagtgaagcagcatatttgaaaggaaagacaacctgttacctgggcctgcaacctccaggcagctcaagagagatgaggcctacagccacagtgggaggggacatggggaatggagatggtccctcaccttcctggggcctcctgctctacgctaccccctcgggagcctcctgtccccagggcaggcccttgccattgttggtcacccggccaagcctctctgcctcaggcgttctcccagaagatctgcccactctcttccccacaccagcccctagagactgaactgaaaaccctcctcagcagggagcctcttctgattaacttcatccagctctggtcacccatcagctcttaaaatgtcaagtggggactgttctttggtatccgttcatttgttgctttgtaaagtgttcccatgtccttgtcttgtctcaagtagattgcaagctcaggagggtagactgggagcccctgagtggagctgctgctcaggccggggctccctgagggcagggctggggctgttctcatactggggctttctgccccaggaccacaccttcctgtcctctctgctcttatggtgccggaggctgcagtgacccaggggcccccaggaatggggaggccgcctgcctcatcgccaggcctcctcacttggccctaaccccagcctttgttttccatttccctcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaa

SEQ ID NO: 4 - DNA, 2503 bp, Homo sapiens
agcccgggcctggccggccgcgtgttcccggagcctcggctgcccgaatggggagcccagagtggcgagcggcacccctccccccgccagccctccgcgggaaggtgacctctcgagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatct

SEQ ID NO: 5 - DNA, 30 bp, Artificial Sequence
  Description of Artificial Sequence Synthetic primer
acgttggatgttagggaagtccttggagtg

SEQ ID NO: 6 - DNA, 30 bp, Artificial Sequence
  Description of Artificial Sequence Synthetic primer
acgttggatggtttcacatagggccaagac

SEQ ID NO: 7 - DNA, 15 bp, Artificial Sequence
  Description of Artificial Sequence Synthetic primer
ctcccctcccccagc

SEQ ID NO: 8 - DNA, 29 bp, Artificial Sequence
  Description of Artificial Sequence Synthetic primer
acgttggatgagactccggcggaagcatt

SEQ ID NO: 9 - DNA, 30 bp, Artificial Sequence
  Description of Artificial Sequence Synthetic primer
acgttggatgaactctctaatcttccgggc

SEQ ID NO: 10 - DNA, 18 bp, Artificial Sequence
  Description of Artificial Sequence Synthetic primer
agggcgggtgacccagca

SEQ ID NO: 11 - DNA, 30 bp, Artificial Sequence
  Description of Artificial Sequence Synthetic primer
acgttggatgggagcacgatggacaaaagc

SEQ ID NO: 12 - DNA, 30 bp, Artificial Sequence
  Description of Artificial Sequence Synthetic primer
acgttggatgatcagaaaacgcacttgccc

SEQ ID NO: 13 - DNA, 19 bp, Artificial Sequence
  Description of Artificial Sequence Synthetic primer
ttgggaaatagcgggaatg

SEQ ID NO: 14 - DNA, 30 bp, Artificial Sequence
  Description of Artificial Sequence Synthetic primer
acgttggatgtcctccacacttctccattc

SEQ ID NO: 15 - DNA, 30 bp, Artificial Sequence
  Description of Artificial Sequence Synthetic primer
acgttggatgcttttccttactcttgactc

SEQ ID NO: 16 - DNA, 20 bp, Artificial Sequence
  Description of Artificial Sequence Synthetic primer
gggcttgtcactgagacagc

SEQ ID NO: 17 - DNA, 30 bp, Artificial Sequence
  Description of Artificial Sequence Synthetic primer
acgttggatggaaacttgtaaaccgagacc

SEQ ID NO: 18 - DNA, 30 bp, Artificial Sequence
  Description of Artificial Sequence Synthetic primer
acgttggatggtacaatccttggtcactcc

SEQ ID NO: 19 - DNA, 21 bp, Artificial Sequence
  Description of Artificial Sequence Synthetic primer
agcaccttaactatagatggt

(19) United States
(12) Patent Application Publication
(10) Pub. No.: US 2014/0336055 A1
(43) Pub. Date: Nov. 13, 2014

(54) GENETIC MARKERS FOR MACULAR DEGENERATION DISORDER TREATMENT

(71) Applicant: SEQUENOM, INC., San Diego, CA (US)

(72) Inventors: Gregory HANNUM, San Diego, CA (US); Karsten E. Schmidt, San Diego, CA (US)

(73) Assignee: SEQUENOM, INC., San Diego, CA (US)

(21) Appl. No.: 14/271,269

(22) Filed: May 6, 2014

Related U.S. Application Data
(60) Provisional application No. 61/820,369, filed on May 7, 2013.

(51) Int. Cl. C12Q 1/68

(52) U.S. Cl. CPC C12Q 1/6883
USPC 506/2; 435/6.11; 506/9

(57) ABSTRACT

Provided in part herein are genetic variations (e.g., single nucleotide polymorphisms) associated with a vascular endothelial growth factor (VEGF) suppression response to an anti-VEGF agent for treatment of a macular degeneration disorder (e.g., age-related macular degeneration (AMD)). Also provided herein are methods for determining a genotype that includes such genetic variations, methods for predicting a VEGF suppression response for a subject according to a genotype, and methods for selecting a treatment suitable for treating a macular degeneration disorder (e.g., wet AMD) for a subject in need thereof according to a genotype.

### RELATED PATENT APPLICATION

This patent application claims the benefit of U.S. provisional application No. 61/820,369, filed on May 7, 2013, entitled GENETIC MARKERS FOR MACULAR DEGENERATION DISORDER TREATMENT, naming Karsten E. Schmidt et al. as inventors and designated by attorney docket no. SEQ-6009-PV. The entire content of the foregoing provisional application is incorporated herein by reference in its entirety, including all text, tables and drawings.

### FIELD

The technology relates in part to genetic variations (e.g., single nucleotide polymorphisms) associated with a vascular endothelial growth factor (VEGF) suppression response to an anti-VEGF agent for treatment of a macular degeneration disorder (e.g., age-related macular degeneration (AMD)).

### BACKGROUND

Age-related macular degeneration (AMD) is the leading cause of irreversible blindness in developed countries. AMD is defined as an abnormality of the retinal pigment epithelium (RPE) that leads to overlying photoreceptor degeneration of the macula and consequent loss of central vision. AMD often leads to a loss of central visual acuity, and can progress in a manner that results in severe visual impairment and blindness. Visual loss in wet AMD is more sudden and may be more severe than in dry AMD. Clinical presentation and course of AMD are variable, and AMD symptoms may present as early as the fifth decade or as late as the ninth decade of life. AMD clinical symptoms range from no visual disturbances in early disease to profound loss of central vision in the advanced late stages of the disease.

In wet AMD, blood vessels invade the macula from the layer under the retina, the choroid, when there is a lack of oxygen in the cells, which is known as choroidal neovascularization (CNV). These new blood vessels are unstable and leak fluid and blood under the retina which causes retinal damage in wet AMD. Vascular endothelial growth factor (VEGF) activity has been associated with ocular blood vessel formation, and agents that inhibit VEGF action have been administered to subjects to reduce blood vessel formation and thereby treat wet AMD. Examples of such agents are anti-VEGF antibodies ranibizumab and bevacizumab, pegylated anti-VEGF aptamer pegaptanib, and immunoadhesins such as aflibercept and conbercept.

### SUMMARY

Provided herein are genetic methods for selecting and/or assessing a treatment regimen for treating an ocular degeneration disorder such as age-related macular degeneration (AMD), and specifically wet AMD. Certain treatments of AMD include administration of an anti vascular endothelial growth factor (anti-VEGF) agent that suppresses VEGF for a period of time in a subject. Genetic methods provided herein can be used to determine (e.g., predict) a VEGF suppression response to an anti-VEGF therapy, and allow for selection and/or assessment of a suitable anti-VEGF treatment and dosing interval according to the determination.

Thus, provided in certain aspects are methods for determining a genotype for a subject, which includes determining a genotype of one or more genetic marker alleles at one or more genetic marker loci associated with (i) a level of ocular VEGF and/or (ii) a VEGF suppression response to an anti-VEGF treatment (e.g., VEGF suppression time), for nucleic acid from a subject. A method provided herein sometimes is performed for nucleic acid from a sample from a subject displaying at least one indicator of wet AMD. A genotype determined sometimes includes one or more single-nucleotide polymorphism (SNP) alleles at each of the SNP loci rs1870377 and rs2071559. A genotype determined sometimes includes one or more SNP alleles in linkage disequilibrium with an allele of rs1870377 or an allele of rs2071559, or an allele of rs1870377 allele and an allele of rs2071559. A VEGF suppression response sometimes is determined for the subject according to the genotype. An AMD treatment regimen and dosing interval sometimes is selected for the subject according to the genotype.

Certain embodiments are described further in the following description, examples, claims and drawings.

### DETAILED DESCRIPTION

Provided herein are genetic methods for selecting and/or assessing an ocular degeneration disorder treatment regimen. Such methods provide several advantages.

For example, many treatment methods for AMD involve administering an anti-VEGF treatment and then adjusting the treatment based on one or more symptoms displayed by the subject, without performing a genetic test. Such treatments often involve multiple patient visits to a health care professional for the purpose monitoring and observing one or more symptoms of the ocular degeneration disorder. Examples of such observation-intensive treatment methods include treat and extend treatment and pro rata needed (PRN) treatments. Genetic methods described herein can provide a health care professional with a prediction of a VEGF suppression response, which can facilitate selection of a therapy and dosing interval individualized for a particular subject, thereby obviating and/or reducing the frequency of patient visits.

Another advantage of genetic methods described herein is that they can be performed using a sample readily obtained from a subject (e.g., using buccal cells from a mouth swab or blood sample). Genetic methods described herein do not require samples obtained by ocular needle injection and aspiration of ocular fluid (e.g., aqueous humor, vitreous humor) for determining a VEGF suppression response. The foregoing advantages of genetic methods described herein can improve quality of, and reduce monetary expenditures associated with, AMD patient care.

### Macular Degeneration Disorders, Indicators and Diagnosis

A macular degeneration disorder sometimes is an age-related macular degeneration (AMD) disorder. Non-limiting examples of AMD disorders are dry AMD and wet AMD. Wet AMD often is associated with choroidal neovascularization (CNV) as described in greater detail herein.

A genotype sometimes is determined for a subject displaying one or more indicators of a macular degeneration disorder (e.g., 1, 2, 3, 4, 5 or more indicators of a macular degeneration disorder). In some embodiments, a genotype is determined for a subject for whom no indicator of a macular degeneration disorder has been observed.

Non-limiting examples dry AMD indicators include (i) the need for brighter light when reading or doing close work, (ii) increasing difficulty adapting to low light levels (e.g., as when entering a dimly lit restaurant), (iii) increasing blurriness of printed words, (iv) decrease in the intensity or brightness of colors, (v) difficulty recognizing faces, (vi) gradual increase in the haziness of central or overall vision, (vi) crooked central vision, (vii) blurred or blind spot in the center of field of vision, (viii) hallucinations of geometric shapes or people, (ix) hyper-pigmentation or hypo-pigmentation of the retinal pigment epithelium (RPE), (x) presence of drusen, and (xi) geographic atrophy of the RPE and photoreceptors. Non-limiting examples of wet AMD indicators include (i) visual distortions, (ii) decreased central vision, (iii) decreased intensity or brightness of colors, (iv) well-defined blurry spot or blind spot in your field of vision, (iv) abrupt onset, (v) rapid worsening, (vi) hallucinations of geometric shapes, animals or people, (vii) hyper-pigmentation or hypo-pigmentation of the retinal pigment epithelium (RPE), (viii) presence of drusen, and (ix) choroidal neovascularization (CNV). Visual distortions sometimes are (i) straight lines appearing wavy or crooked, (ii) objects (e.g., doorway or street sign) appearing lopsided, and/or (iii) objects appearing smaller or farther away than they really are. Such indicators may be present for one or both eyes of a subject.

A genotype sometimes is determined for a subject diagnosed with a macular degeneration disorder (e.g., wet AMD, CNV). Non-limiting examples of diagnostics for dry AMD and wet AMD include (i) central vision defect testing, (ii) examination of the back of the eye, (iii) angiogram (e.g., fluorescein angiogram); and (iv) optical coherence tomography. An Amsler grid can be used to test for defects in central vision, and macular degeneration can cause the straight lines in the grid to appear faded, broken or distorted. Presence of fluid or blood identified in an examination of the back of the eye, in which pupils are dilated and an optical device scans the back of the eye, can diagnose wet AMD. In a fluorescein angiogram, a colored dye is injected into an arm vein, the dye travels to the blood vessels in the eye, a camera images the blood vessels as the dye travels through the blood vessels, and camera images show the presence or absence of blood vessel or retinal abnormalities that may be associated with wet macular degeneration. In optical coherence tomography, imaging displays detailed cross-sectional images of the eye and identifies retinal abnormalities, such as retina swelling or leaking blood vessels.

Early, intermediate or advanced stage dry AMD or wet AMD can be diagnosed using diagnostic methods based in part on size of drusen and level of breakdown in macular cells. For example, early AMD is characterized by drusen (greater than 63 um) and hyper-pigmentation or hypo-pigmentation of the retinal pigment epithelium (RPE). Intermediate AMD is characterized by the accumulation of focal or diffuse drusen (greater than 125 um) and hyper-pigmentation or hypo-pigmentation of the RPE. Advanced dry AMD is associated with vision loss due to geographic atrophy of the RPE and photoreceptors. Advanced wet AMD is associated with choroidal neovascularization (CNV), which is observed as neovascular choriocapillary invasion across Bruch's membrane into the RPE and photoreceptor layers. Certain environmental and genetic factors can be taken into account when diagnosing an AMD condition, including without limitation, one or more of age, race (e.g., higher prevalence in Caucasian and African descent populations), diet (e.g., fat intake), smoking history, body mass index (e.g., obesity), hypertension, cholesterol level (e.g., elevated cholesterol), oxidative stress, light exposure history, fibulin-5 mutation, CFHR1 deletion, CFHR3 deletion, and the like.

### Genotypes

Genotypes can be determined for one or more genetic markers in nucleic acid from a subject, which are described in greater detail hereafter.

Nucleic Acid

A genotype can be determined using nucleic acid. Nucleic acid used to determine a genotype often is from a suitable sample from a subject, and sometimes is a processed version thereof. A subject can be any living or non-living organism, including but not limited to a human, a non-human animal, a plant, a bacterium, a fungus or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any suitable age (e.g., an embryo, a fetus, infant, child, adult).

Nucleic acid utilized for determining a genotype sometimes is cellular nucleic acid or processed version thereof. Cellular nucleic acid often is isolated from a source having intact cells. Non-limiting examples of sources for cellular nucleic acid are blood cells, tissue cells, organ cells, tumor cells, hair cells, skin cells, and bone cells. Nucleic acid sometime is circulatory extracellular nucleic acid, or cell-free nucleic acid, or a processed version thereof. Such nucleic acid sometimes is from an acellular source (e.g., nucleic acid from urine or a cell-free blood component (e.g., plasma, serum)). Nucleic acid may be isolated from any type of suitable biological specimen or sample (e.g., a test sample). Non-limiting examples of specimens include fluid or tissue from a subject, including, without limitation, cerebrospinal fluid, spinal fluid, lavage fluid (e.g., bronchoalveolar, gastric, peritoneal, ductal, ear, arthroscopic), urine, feces, sputum, saliva, nasal mucous, prostate fluid, lavage, semen, lymphatic fluid, bile, tears, sweat, breast milk, breast fluid, biopsy sample (e.g., cancer biopsy), cell or tissue sample (e.g., from the liver, lung, spleen, pancreas, colon, skin, bladder, eye, brain, esophagus, head, neck, ovary, testes, prostate, the like or combination thereof). A sample sometimes includes buccal cells (e.g., from a mouth swab). In some embodiments, a biological sample may be blood and sometimes a blood fraction (e.g., plasma or serum). As used herein, the term “blood” encompasses whole blood or any fractions of blood, such as serum and plasma as conventionally defined, for example. Blood or fractions thereof often comprise nucleosomes (e.g., maternal and/or fetal nucleosomes). Nucleosomes comprise nucleic acids and are sometimes cell-free or intracellular. Blood also comprises buffy coats. Buffy coats sometimes are isolated by utilizing a ficoll gradient. Buffy coats can comprise white blood cells (e.g., leukocytes, T-cells, B-cells, platelets, and the like). In some embodiments, buffy coats comprise maternal and/or fetal nucleic acid. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation. A fluid or tissue sample from which nucleic acid is extracted may be acellular (e.g., cell-free). In some embodiments, a fluid or tissue sample may contain cellular elements or cellular remnants. In some embodiments cancer cells may be included in a sample.

Any suitable method known in the art for obtaining a sample from a subject can be utilized. Any suitable method known in the art for isolating and/or purifying nucleic acid from the sample can be utilized. Obtaining a sample sometimes includes obtaining a sample directly (e.g., collecting a sample, e.g., a test sample) from a subject, and sometimes includes obtaining a sample from another who has collected a sample from a subject. Obtaining nucleic acid includes isolating nucleic acid from a sample, and sometimes includes obtaining nucleic acid from another who has isolated nucleic acid from a sample.

Nucleic acid from a sample can be processed by a suitable method prior to, or as part of, determining a genotype. A suitable combination of nucleic acid modification processes known in the art (e.g., described herein) may be utilized.

Nucleic acid sometimes is subjected to a fragmentation or cleavage process, which may be a specific cleavage process or a non-specific fragmentation process. Non-limiting examples of fragmentation and cleavage processes include physical fragmentation processes, chemical fragmentation processes and enzymatic cleavage process (e.g., a process making use of one or more restriction enzymes and/or nuclease enzymes).

Nucleic acid sometimes is subjected to a methylation-specific modification process. Non-limiting examples of methylation-specific modification processes, which also can be used for detecting and/or quantifying a methylation state of a nucleic acid, include bisulfite treatment of DNA, bisulfite sequencing, methylation specific PCR (MSP), quantitative methylation specific PCR (QPSP), combined bisulfite restriction analysis (COBRA), methylation-sensitive single nucleotide primer extension (Ms-SNuPE), MethylLight, methylation pyrosequencing, immunoprecipitation with 5-Methyl Cytosine (MeDIP), Methyl CpG Immunoprecipitation (MCIp; e.g., use of an antibody that specifically binds to a methyl-CpG binding domain (MBD) of a MBD2 methyl binding protein (MBD-Fc) for immunoprecipitation of methylated or unmethylated DNA), methyl-dependent enzyme digestion with McrBC, and processes disclosed in International Application Publication No. WO 2011/034631 published on Mar. 24, 2011 (International Application No. PCT/US2010/027879 filed on Mar. 18, 2010) and in International Application Publication No. WO 2012/149339 published on Nov. 1, 2012 (International Application No. PCT/US2012/035479 filed on Apr. 27, 2012).

In some embodiments, nucleic acid is subjected to an amplification process. Non-limiting examples of amplification processes include polymerase chain reaction (PCR); ligation amplification (or ligase chain reaction (LCR)); amplification methods based on the use of Q-beta replicase or template-dependent polymerase (see US Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, the like and combinations thereof.

Nucleic acid sometimes is processed by a method that incorporates or appends a detectable label or tag into or to the nucleic acid. Non-limiting examples of detectable labels include fluorescent labels such as organic fluorophores, lanthanide fluorophores (chelated lanthanides; dipicolinate-based Terbium (III) chelators), transition metal-ligand complex fluorophores (e.g., complexes of Ruthenium, Rhenium or Osmium); quantum dot fluorophores, isothiocyanate fluorophore derivatives (e.g., FITC, TRITC), succinimidyl ester fluorophores (e.g., NHS-fluorescein), maleimide-activated fluorophores (e.g., fluorescein-5-maleimide), and amidite fluorophores (e.g., 6-FAM phosphoramidite); radioactive isotopes (e.g., I-125, I-131, S-35, P-31, P-32, C-14, H-3, Be-7, Mg-28, Co-57, Zn-65, Cu-67, Ge-68, Sr-82, Rb-83, Tc-95m, Tc-96, Pd-103, Cd-109, and Xe-127); light scattering labels (e.g., light scattering gold nanorods, resonance light scattering particles); an enzymic or protein label (e.g., green fluorescence protein (GFP), peroxidase); or other chromogenic label or dye (e.g., cyanine). Non-limiting examples of organic fluorophores include xanthene derivatives (e.g., fluorescein, rhodamine, Oregon green, eosin, Texas red); cyanine derivatives (e.g., cyanine, indocarbocyanine, oxacarbocyanine, thiacarbocyanine, merocyanine); naphthalene derivatives (dansyl, prodan derivatives); coumarin derivatives; oxadiazole derivatives (e.g., pyridyloxazole, nitrobenzoxadiazole, benzoxadiazole); pyrene derivatives (e.g., cascade blue); oxazine derivatives (e.g., Nile red, Nile blue, cresyl violet, oxazine 170); acridine derivatives (e.g., proflavin, acridine orange, acridine yellow); arylmethine derivatives (e.g., auramine, crystal violet, malachite green); and tetrapyrrole derivatives (e.g., porphin, phtalocyanine, bilirubin).

Nucleic acid sometimes is processed by a method that incorporates or appends a capture agent or mass-distinguishable label into or to the nucleic acid. Non-limiting examples of capture agents include biotin, avidin and streptavidin. Any suitable mass-distinguishable label known in the art can be utilized, and mass-distinguishable labels that permit multiplexing in a particular mass window for mass spectrometry analysis sometimes are utilized. Methods for incorporating or appending a capture agent or mass-distinguishable label into or to a nucleic acid are known in the art, and sometimes include amplifying sample nucleic acid using one or more amplification primers that include a capture agent or mass-distinguishable label.

Nucleic acid isolated from a sample may be modified by a method used to process it, and a processing method may or may not result in a modified nucleic acid. Any suitable type of nucleic acid can be used to determine a genotype. Non-limiting examples of nucleic acid that can be utilized for genotyping include deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), DNA or RNA analogs (e.g., containing base analogs, sugar analogs and/or a non-native backbone and the like), RNA/DNA hybrids and polyamide nucleic acids (PNAs).

A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell, in certain embodiments. A nucleic acid in some embodiments can be from a single chromosome (e.g., a nucleic acid sample may be from one chromosome of a sample obtained from a diploid organism). The term also may include, as equivalents, derivatives, variants and analogs of RNA or DNA synthesized from nucleotide analogs, single-stranded (e.g., “sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base thymine is replaced with uracil.

Genetic Markers

A genotype generally includes the identity of a nucleotide or nucleotides present at a genetic location (locus). A genetic locus sometimes is referred to as a genetic marker and sometimes is polymorphic when the nucleotide or nucleotides a the locus vary among individuals in a population. A nucleotide or nucleotide sequence at a genetic locus or marker sometimes is referred to as an allele (e.g., a polynucleotide sequence at a locus). An allele sometimes is referred to as a minor allele or major allele. An allele occurring with less frequency than another allele, referred to as a minor allele, often occurs at a frequency in a population greater than the frequency of the occurrence of a spontaneous mutation. A minor allele frequency sometimes is about 5% or greater in a population (e.g., about 6% or more, 7% or more, 8% or more, 9% or more, 10% or more, 11% or more, 12% or more, 15% or more, 20% or more, 25% or more, 30% or more, 35% or more, 40% or more, 45% or more up to 49.9%). A subject may be homozygous for a genetic marker allele (i.e., same alleles on chromosomes) and sometimes is heterozygous for a genetic marker allele (i.e., different alleles on chromosomes).

A genetic locus sometimes includes one nucleotide, as in the case of a single nucleotide polymorphism (SNP), for example. A genetic locus sometimes includes two or more nucleotides, and sometimes is about 2 contiguous nucleotides to about 100 contiguous nucleotides in length (e.g., about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 contiguous nucleotides). Non-limiting examples of genetic loci types having more than one nucleotide include restriction fragment length polymorphisms (RFLP), simple sequence length polymorphisms (SSLP), amplified fragment length polymorphisms (AFLP), random amplification of polymorphic DNAs (RAPD), variable number tandem repeats (VNTR), microsatellite polymorphisms, simple sequence repeats (SSR), short tandem repeats (STR), single feature polymorphisms (SFP), diversity array technology markers (DArT) and restriction site associated DNA markers (RAD markers).

A genotype can include an allele for one or more genetic markers, and sometimes includes allele sequence information informative as to whether a subject is heterozygous or homozygous for allele(s) at each genetic locus or marker. A genotype sometimes includes alleles for about 2 or more genetic markers, and sometimes includes alleles for about 2 to about 100 genetic markers (e.g., about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 genetic markers). A genotype sometimes includes alleles for only one type of genetic marker (e.g., only SNPs) and sometimes includes alleles for different types of genetic markers (e.g., SNPs and STRs).

The identity of a nucleotide or polynucleotide sequence at a genetic locus for a genotype sometimes is for one chromosome, and sometimes is for two chromosomes, (e.g., the nucleotide or nucleotides at a genetic locus may be the same or different on each chromosome). The identity of a nucleotide or polynucleotide sequence at a genetic locus for a genotype sometimes is for one nucleic acid strand for single-stranded or double-stranded nucleic acid, and sometimes is for two nucleic acid strands for double-stranded nucleic acid. A genotype sometimes includes the identity of a nucleotide or polynucleotide sequence at two or more genetic loci or markers on one chromosome, and such genotypes sometimes are presented as a haplotype (i.e., a combination of alleles at adjacent loci on a chromosome that are inherited together). Genetic marker loci in a genotype sometimes are located in a single chromosome, and sometimes are located within about 0.5 kilobases (kb) to about 100 kb (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 kb).

A genetic marker allele reported in a genotype sometimes is associated with an ocular vascular endothelial growth factor (VEGF) suppression response to a treatment that suppresses ocular VEGF. Ocular VEGF in the suppression response sometimes is retinal VEGF. An ocular VEGF suppression response sometimes is an ocular VEGF suppression response time. Non-limiting examples of ocular VEGF suppression times include about 2 days until a baseline ocular VEGF level is restored after treatment with a VEGF suppressor to about 120 days until a baseline ocular VEGF level is restored after treatment with a VEGF suppressor (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 115 days until a baseline VEGF level is restored). A baseline ocular VEGF level often is an ocular VEGF level prior to treatment with a VEGF suppressor, and a baseline ocular VEGF level sometimes is a retinal VEGF level, aqueous humor VEGF level, and/or vitreous humor VEGF level. Restoration of an ocular VEGF baseline level generally is an ocular VEGF level within about 10% or less of an ocular VEGF baseline level for the subject prior to treatment with an ocular VEGF suppressor (e.g., about 9%, 8%, 7%, 6%, 5%, 4%, 3%, 2% or 1% or less of the VEGF baseline level). A baseline ocular VEGF level sometimes is about 10 picograms per milliliter (pg/ml) VEGF to about 500 pg/ml VEGF (e.g., about 15, 20, 25, 30, 35, 40, 45, 50, 55, 60, 65, 70, 75, 80, 85, 90, 95, 100, 105, 110, 115, 120, 130, 140, 150, 160, 170, 180, 190, 200, 250, 300, 350, 400, 450 pg/ml VEGF). An ocular VEGF level suppressed by a VEGF suppressor sometimes is to about 9 pg/ml of ocular VEGF or less (e.g., about 8, 7, 6, 5, 4, 3, 2 or 1 pg/ml or less). Suitable methods for measuring ocular VEGF levels and ocular VEGF suppression times are known in the art (e.g., Muether et al., Am. Acad. Ophthalmology 119(10): 2082-2086. (2012)).

A genetic marker allele reported in a genotype sometimes is associated with a relatively short ocular VEGF suppression time, sometimes is associated with a relatively long ocular VEGF suppression time, or sometimes is associated with a relatively average ocular VEGF suppression time (e.g., mean, median, mode ocular VEGF suppression time) for a population. A relatively short ocular VEGF suppression time sometimes is at least about 5 days less (e.g., about 15, 14, 13, 12, 11, 10, 9, 8, 7 or 6 days less) than an average VEGF suppression time (e.g., mean, median, mode) in a population. A relatively long VEGF ocular suppression time sometimes is at least about 5 days more (e.g., about 6, 7, 8, 9, 10, 11, 12, 13, 14, 15 days more) than the average VEGF suppression time (e.g., mean, median, mode) for a population. A relatively average ocular VEGF suppression time sometimes is within about 5 days (e.g., about 4, 3, 2, 1 days) of the average VEGF suppression time (e.g., mean, median, mode) for a population.

A genetic marker allele sometimes is associated with an ocular VEGF suppression time for a particular class of VEGF suppressors or particular VEGF suppressor. Examples of VEGF suppressor agents and classes of agents are described herein. Non-limiting examples of classes of VEGF suppressors include agents that (i) bind to, cleave or inhibit production of a VEGF, (ii) bind to, cleave or inhibit production of a VEGFR and (iii) bind to, cleave or inhibit production of a cytoplasmic protein participating in VEGFR signaling pathway (e.g., a tyrosine protein kinase). Non-limiting examples of ocular VEGF suppressor agents include antibody, aptamer, ankyrin repeat protein and recombinant protein agents. Non-limiting examples of ocular VEGF suppressor agents include ranibizumab, bevacizumab, pegaptanib, aflibercept, conbercept or an agent that elicits an average (e.g., mean, median, mode) ocular VEGF suppression time similar to the average ocular VEGF suppression time elicited by ranibizumab, bevacizumab, pegaptanib or aflibercept. A similar average ocular VEGF suppression time generally is within about 25% or less (e.g., about 20% or less, 15% or less, 10% or less, 5% or less) of the average ocular VEGF suppression time elicited by ranibizumab, bevacizumab, pegaptanib or aflibercept in a population.

A genetic marker allele sometimes is associated with an ocular VEGF suppression response in a particular population. A population sometimes is ethnically diverse, and sometimes is predominantly composed of an ethnic group (e.g., Caucasian, Asian, Asian-American, African, African-American, Hispanic and the like). Degree of association between a particular genetic marker allele with an ocular VEGF suppression response can vary between populations. When a genetic marker is located in a conserved genomic region (e.g., the genomic region for VEGFR-2 generally is conserved), degree of association for the marker with an ocular VEGF suppression response often is low.

A genotype in some embodiments includes one or more alleles for two or more SNP markers. Non-limiting examples of SNP loci include loci chosen from (i) rs1870377, rs2071559, rs3025033, rs3025039, a SNP allele in linkage disequilibrium with an allele of one or more of the foregoing SNP loci, or combination thereof; (ii) rs1870377, rs2071559, rs3025033, rs3025039, rs2305948, a SNP allele in linkage disequilibrium with an allele of one or more of the foregoing SNP loci, or combination thereof; or (iii) rs1870377, rs2071559, rs3025033, rs3025039, rs2305948, a SNP allele in linkage disequilibrium with an allele of one or more of the foregoing SNP loci, a SNP allele in a polynucleotide that encodes a polypeptide in a VEGF signaling pathway, a SNP allele in a first polynucleotide in operable connection with a second polynucleotide that encodes a polypeptide in a VEGF signaling pathway, or combination thereof. In some embodiments, a genotype comprises one or more SNP alleles at each of the SNP loci comprising rs1870377 and rs2071559. In certain embodiments, a genotype comprises one or more SNP alleles at each of the SNP loci consisting of rs1870377, rs2071559 and one or more SNP alleles in linkage disequilibrium with an allele of rs1870377 or an allele of rs2071559, or an allele of rs1870377 allele and an allele of rs2071559. In some embodiments, a genotype comprises one or more SNP alleles at each of the SNP loci consisting of rs1870377 and rs2071559. In certain embodiments, the presence or absence of a thymine allele at rs1870377, or an adenine allele at rs1870377 allele, or a thymine allele and an adenine allele at rs1870377, is determined. In some embodiments, the presence or absence of a guanine allele at rs2071559 or an adenine allele at rs2071559, or a guanine allele and an adenine allele at rs2071559, is determined.

Loci rs1870377, rs2071559 and rs2305948 are within genomic DNA comprising an open reading frame that encodes vascular endothelial growth factor (VEGF) receptor 2 (VEGFR-2). Human VEGFR-2 genomic DNA is deposited and includes the nucleotide sequence of SEQ ID NO: 1. Loci rs1870377, rs2071559 and rs2305948 are at positions 28330, 47722 and 34914 in SEQ ID NO: 1, respectively. Provided as SEQ ID NO: 2 is a human VEGFR-2 complementary DNA nucleotide sequence.

Loci rs3025033 and rs3025039 are within genomic DNA comprising an open reading frame that encodes vascular endothelial growth factor A (VEGF-A). Human VEGF-A genomic DNA is deposited and includes the nucleotide sequence of SEQ ID NO: 3. Loci rs3025033 and rs3025039 are at positions 13130 and 14591 in SEQ ID NO: 3, respectively. Provided as SEQ ID NO: 4 is a human VEGF-A complementary DNA nucleotide sequence.

A SNP allele in linkage disequilibrium with another SNP allele sometimes is characterized as having an R-squared assessment of linkage disequilibrium of about 0.3 or greater (e.g., an R-squared value of 0.30 or greater, 0.35 or greater, 0.40 or greater, 0.45 or greater, 0.50 or greater, 0.55 or greater, 0.60 or greater, 0.65 or greater, 0.70 or greater, 0.75 or greater, 0.80 or greater, 0.85 or greater, 0.90 or greater, 0.95 or greater). A SNP allele in linkage disequilibrium with another SNP allele sometimes is characterized as having a D-prime assessment of linkage disequilibrium of about 0.6 or greater (e.g., a D-prime assessment of 0.60 or greater, 0.65 or greater, 0.70 or greater, 0.75 or greater, 0.80 or greater, 0.85 or greater, 0.90 or greater, 0.95 or greater). R-squared and D-prime assessments of linkage disequilibrium are known in the art.

In some embodiments, a SNP allele in linkage disequilibrium with an allele of rs1870377 is chosen from an allele of rs7677779, rs13136007, rs58415820, rs2305946, rs3816584, rs6838752, rs2219471, rs1870378, rs1870379, rs35624269, rs17085267, rs17085265, rs17085262, rs13127286, rs10016064, rs4864532, rs1458830, rs17709898, rs11940163, rs7671745, rs6846151, rs17085326 and rs7673274.

In certain embodiments, a SNP allele in linkage disequilibrium with an allele of rs2071559 is chosen from an allele of rs28695311, rs2219469, rs6837695, rs4864956, rs7686613, rs13143757, rs58309017, rs2412637, rs7679993, rs7680198, rs7675314, rs1458829, rs7696256, rs17712245, rs1380057, rs1580217, rs1580216, rs2125493, rs1547512, rs1547511, rs62304733, rs6554237, rs17081840, rs7667298, rs11936364, rs9994560, rs1350542, rs1350543, rs55713360, rs1380069, rs11722032, rs36104862, rs12502008, rs7693746, rs1380061, rs1380062, rs1380063, rs1380064, rs4241992, rs4864957, rs4864958, rs10517342, rs7662807, rs75208589, rs74866484, rs11935575, rs1458822, rs9312658, rs73236109, rs1903068, rs4516787, rs6816309, rs6833067, rs6811163, rs1458823, rs4356965, rs12331507, rs12646502, rs1551641, rs1551642, rs1551643, rs1551645, rs17773813, rs78025085, rs6842494, rs12331597, rs17773240, rs28411232, rs12331471, rs9312655, rs10012589, rs10012701, rs9312656, rs9312657, rs12505096, rs12498317, rs28838369, rs28680424, rs73236111, rs9997685, rs1551644, rs17711320, rs10517343, rs13134246, rs13134290, rs13134291, rs13134452, rs10020668, rs10013228, rs28584303, rs12331538, rs35729366, rs28517654, rs73236106, rs17711225, rs9284955, rs1380068, rs1350545, rs9998950, rs62304743, rs2239702, rs41408948, rs73236104 and rs10026340.

In some embodiments, a SNP allele in linkage disequilibrium with an allele of rs3025033 is chosen from an allele of rs3025030, rs3025029, rs3025039, rs3025040, rs6899540, rs78807370, rs73416585, rs9472126 and rs12204488. In certain embodiments, a SNP allele in linkage disequilibrium with an allele of rs3025039 is chosen from an allele of rs3025039, rs3025030, rs3025029, rs3025033, rs3025040, rs6899540, rs78807370, rs73416585 and rs9472126. In some embodiments a SNP allele in linkage disequilibrium with an allele of rs2305948 is chosen from rs2305949 and rs34945396.

The following Table A provides genomic polynucleotide positions corresponding to selected SNP positions described herein.

TABLE A
SNP positions in SEQ ID NO: 1 and
SEQ ID NO: 3

| SNP | Genomic | |
|---|---|---|
| rsID | polynucleotide | position |
| 3025029 | VEGFA (SEQ ID NO: 3) | 12611 |
| 3025030 | VEGFA (SEQ ID NO: 3) | 12642 |
| 3025033 | VEGFA (SEQ ID NO: 3) | 13130 |
| 3025039 | VEGFA (SEQ ID NO: 3) | 14591 |
| 3025040 | VEGFA (SEQ ID NO: 3) | 15106 |
| 7671745 | VEGFR2 (SEQ ID NO: 1) | 12192 |
| 11940163 | VEGFR2 (SEQ ID NO: 1) | 12671 |
| 13127286 | VEGFR2 (SEQ ID NO: 1) | 12672 |
| 17709898 | VEGFR2 (SEQ ID NO: 1) | 13079 |
| 1458830 | VEGFR2 (SEQ ID NO: 1) | 13358 |
| 17085262 | VEGFR2 (SEQ ID NO: 1) | 14497 |
| 17085265 | VEGFR2 (SEQ ID NO: 1) | 14508 |
| 17085267 | VEGFR2 (SEQ ID NO: 1) | 15218 |
| 35624269 | VEGFR2 (SEQ ID NO: 1) | 15451 |
| 4864532 | VEGFR2 (SEQ ID NO: 1) | 15760 |
| 2219471 | VEGFR2 (SEQ ID NO: 1) | 16515 |
| 6838752 | VEGFR2 (SEQ ID NO: 1) | 19457 |
| 3816584 | VEGFR2 (SEQ ID NO: 1) | 19921 |
| 2305946 | VEGFR2 (SEQ ID NO: 1) | 19961 |
| 58415820 | VEGFR2 (SEQ ID NO: 1) | 20790 |
| 1870379 | VEGFR2 (SEQ ID NO: 1) | 21660 |
| 1870378 | VEGFR2 (SEQ ID NO: 1) | 21809 |
| 7677779 | VEGFR2 (SEQ ID NO: 1) | 23040 |
| 13136007 | VEGFR2 (SEQ ID NO: 1) | 24362 |
| 10016064 | VEGFR2 (SEQ ID NO: 1) | 25561 |
| 1870377 | VEGFR2 (SEQ ID NO: 1) | 28330 |
| 6846151 | VEGFR2 (SEQ ID NO: 1) | 29646 |
| 7673274 | VEGFR2 (SEQ ID NO: 1) | 31075 |
| 17085326 | VEGFR2 (SEQ ID NO: 1) | 32732 |
| 2305948 | VEGFR2 (SEQ ID NO: 1) | 34914 |
| 2305949 | VEGFR2 (SEQ ID NO: 1) | 35812 |
| 34945396 | VEGFR2 (SEQ ID NO: 1) | 38140 |
| 1380057 | VEGFR2 (SEQ ID NO: 1) | 45031 |
| 73236104 | VEGFR2 (SEQ ID NO: 1) | 46310 |
| 12502008 | VEGFR2 (SEQ ID NO: 1) | 46398 |
| 7667298 | VEGFR2 (SEQ ID NO: 1) | 47087 |
| 9994560 | VEGFR2 (SEQ ID NO: 1) | 47183 |
| 41408948 | VEGFR2 (SEQ ID NO: 1) | 47381 |
| 55713360 | VEGFR2 (SEQ ID NO: 1) | 47423 |
| 28695311 | VEGFR2 (SEQ ID NO: 1) | 47461 |
| 2239702 | VEGFR2 (SEQ ID NO: 1) | 47495 |
| 2071559 | VEGFR2 (SEQ ID NO: 1) | 47722 |
| 28517654 | VEGFR2 (SEQ ID NO: 1) | 48824 |

### Genotype Determination Processes

A genotype for nucleic acid from a subject can be determined using any suitable process known in the art. Determining a genotype sometimes includes obtaining a genotype for a subject already stored in a database. A genotype sometimes is obtained from a database using a computer, microprocessor, memory or combination thereof. Determining a genotype sometimes includes obtaining the genotype from another who already has performed a genetic analysis on nucleic acid from the subject. Determining a genotype sometimes includes determining the nucleotide or polynucleotide sequence of one or more genetic marker alleles in nucleic acid from a subject. Determining a genotype sometimes comprises analyzing a nucleic acid from the subject, or analyzing a nucleic acid derived from nucleic acid from the subject. Any suitable nucleic acid analysis process that provides a genotype can be utilized, as described in greater detail herein (e.g., a sequencing process or mass spectrometry process). Determining a genotype sometimes includes obtaining nucleic acid from a subject, which sometimes includes one or more of isolating a sample from the subject, isolating nucleic acid from the sample, and processing the nucleic acid prior to genotype analysis.

Any suitable technology can be used to determine a genotype for a nucleic acid. Determining a genotype sometimes includes detecting and/or quantifying the genotype. Non-limiting examples of technologies that can be utilized to determine a genotype include mass spectrometry, amplification (e.g., digital PCR, quantitative polymerase chain reaction (qPCR)), sequencing (e.g., nanopore sequencing, base extension sequencing (e.g., single base extension sequencing), sequencing by synthesis), array hybridization (e.g., microarray hybridization; gene-chip analysis), flow cytometry, gel electrophoresis (e.g., capillary electrophoresis), cytofluorimetric analysis, fluorescence microscopy, confocal laser scanning microscopy, laser scanning cytometry, affinity chromatography, manual batch mode separation, electric field suspension, the like and combinations of the foregoing. Further detail is provided hereafter for certain genotype detection and/or quantification technologies.

Mass Spectrometry

In some embodiments, mass spectrometry is used to detect and/or quantify nucleic acid fragments. Mass spectrometry methods typically are used to determine the mass of a molecule, such as a nucleic acid fragment. In some embodiments, mass spectrometry is used in conjunction with another detection, enrichment and/or separation method known in the art or described herein such as, for example, MassARRAY, primer extension (e.g., MASSEXTEND), probe extension, methods using mass modified probes and/or primers, and the like. The relative signal strength, e.g., mass peak on a spectra, for a particular nucleic acid fragment can indicate the relative population of the fragment species amongst other nucleic acids in the sample (see e.g., Jurinke et al. (2004) Mol. Biotechnol. 26, 147-164).

Mass spectrometry generally works by ionizing chemical compounds to generate charged molecules or molecule fragments and measuring their mass-to-charge ratios. A typical mass spectrometry procedure involves several steps, including (1) loading a sample onto a mass spectrometry instrument followed by vaporization, (2) ionization of the sample components by any one of a variety of methods (e.g., impacting with an electron beam), resulting in charged particles (ions), (3) separation of ions according to their mass-to-charge ratio in an analyzer by electromagnetic fields, (4) detection of ions (e.g., by a quantitative method), and (5) processing of ion signals into mass spectra.

Mass spectrometry methods are known, and include without limitation quadrupole mass spectrometry, ion trap mass spectrometry, time-of-flight mass spectrometry, gas chromatography mass spectrometry and tandem mass spectrometry can be used with a method described herein. Processes associated with mass spectrometry are generation of gas-phase ions derived from the sample, and measurement of ions. Movement of gas-phase ions can be precisely controlled using electromagnetic fields generated in the mass spectrometer, and movement of ions in these electromagnetic fields is proportional to the mass to charge ratio (m/z) of each ion, which forms the basis of measuring m/z and mass. Movement of ions in these electromagnetic fields allows for containment and focusing of the ions which accounts for high sensitivity of mass spectrometry. During the course of m/z measurement, ions are transmitted with high efficiency to particle detectors that record the arrival of these ions. The quantity of ions at each m/z is demonstrated by peaks on a graph where the x axis is m/z and the y axis is relative abundance. Different mass spectrometers have different levels of resolution (i.e., the ability to resolve peaks between ions closely related in mass). Resolution generally is defined as R=m/delta m, where m is the ion mass and delta m is the difference in mass between two peaks in a mass spectrum. For example, a mass spectrometer with a resolution of 1000 can resolve an ion with a m/z of 100.0 from an ion with a m/z of 100.1.

Certain mass spectrometry methods can utilize various combinations of ion sources and mass analyzers which allows for flexibility in designing customized detection protocols. In some embodiments, mass spectrometers can be programmed to transmit all ions from the ion source into the mass spectrometer either sequentially or at the same time. In some embodiments, a mass spectrometer can be programmed to select ions of a particular mass for transmission into the mass spectrometer while blocking other ions.

Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Mass analyzers include, for example, a quadrupole mass filter, ion trap mass analyzer and time-of-flight mass analyzer.

An ion formation process generally is a starting point for mass spectrum analysis. Several ionization methods are available and the choice of ionization method depends on the sample used for analysis. For example, for the analysis of polypeptides a relatively gentle ionization procedure such as electrospray ionization (ESI) can be desirable. For ESI, a solution containing the sample is passed through a fine needle at high potential which creates a strong electrical field resulting in a fine spray of highly charged droplets that is directed into the mass spectrometer. Other ionization procedures include, for example, fast-atom bombardment (FAB) which uses a high-energy beam of neutral atoms to strike a solid sample causing desorption and ionization.

Matrix-assisted laser desorption ionization (MALDI) is a method in which a laser pulse is used to strike a sample that has been crystallized in an UV-absorbing compound matrix (e.g., 2,5-dihydroxybenzoic acid, alpha-cyano-4-hydroxycinammic acid, 3-hydroxypicolinic acid (3-HPA), di-ammoniumcitrate (DAC) and combinations thereof). Other ionization procedures known in the art include, for example, plasma and glow discharge, plasma desorption ionization, resonance ionization, and secondary ionization.

A variety of mass analyzers are available that can be paired with different ion sources. Different mass analyzers have different advantages as known in the art and as described herein. The mass spectrometer and methods chosen for detection depends on the particular assay, for example, a more sensitive mass analyzer can be used when a small amount of ions are generated for detection. Several types of mass analyzers and mass spectrometry methods are described below.

Ion mobility mass (IM) spectrometry is a gas-phase separation method. IM separates gas-phase ions based on their collision cross-section and can be coupled with time-of-flight (TOF) mass spectrometry. IM-MS methods are known in the art.

Quadrupole mass spectrometry utilizes a quadrupole mass filter or analyzer. This type of mass analyzer is composed of four rods arranged as two sets of two electrically connected rods. A combination of rf and dc voltages are applied to each pair of rods which produces fields that cause an oscillating movement of the ions as they move from the beginning of the mass filter to the end. The result of these fields is the production of a high-pass mass filter in one pair of rods and a low-pass filter in the other pair of rods. Overlap between the high-pass and low-pass filter leaves a defined m/z that can pass both filters and traverse the length of the quadrupole. This m/z is selected and remains stable in the quadrupole mass filter while all other m/z have unstable trajectories and do not remain in the mass filter. A mass spectrum results by ramping the applied fields such that an increasing m/z is selected to pass through the mass filter and reach the detector. In addition, quadrupoles can also be set up to contain and transmit ions of all m/z by applying a rf-only field. This allows quadrupoles to function as a lens or focusing system in regions of the mass spectrometer where ion transmission is needed without mass filtering.

A quadrupole mass analyzer, as well as the other mass analyzers described herein, can be programmed to analyze a defined m/z or mass range. Since the desired mass range of nucleic acid fragment is known, in some instances, a mass spectrometer can be programmed to transmit ions of the projected correct mass range while excluding ions of a higher or lower mass range. The ability to select a mass range can decrease the background noise in the assay and thus increase the signal-to-noise ratio. Thus, in some instances, a mass spectrometer can accomplish a separation step as well as detection and identification of certain mass-distinguishable nucleic acid fragments.

Ion trap mass spectrometry utilizes an ion trap mass analyzer. Typically, fields are applied such that ions of all m/z are initially trapped and oscillate in the mass analyzer. Ions enter the ion trap from the ion source through a focusing device such as an octapole lens system. Ion trapping takes place in the trapping region before excitation and ejection through an electrode to the detector. Mass analysis can be accomplished by sequentially applying voltages that increase the amplitude of the oscillations in a way that ejects ions of increasing m/z out of the trap and into the detector. In contrast to quadrupole mass spectrometry, all ions are retained in the fields of the mass analyzer except those with the selected m/z. Control of the number of ions can be accomplished by varying the time over which ions are injected into the trap.

Time-of-flight mass spectrometry utilizes a time-of-flight mass analyzer. Typically, an ion is first given a fixed amount of kinetic energy by acceleration in an electric field (generated by high voltage). Following acceleration, the ion enters a field-free or “drift” region where it travels at a velocity that is inversely proportional to its m/z. Therefore, ions with low m/z travel more rapidly than ions with high m/z. The time required for ions to travel the length of the field-free region is measured and used to calculate the m/z of the ion.

Gas chromatography mass spectrometry often can a target in real-time. The gas chromatography (GC) portion of the system separates the chemical mixture into pulses of analyte and the mass spectrometer (MS) identifies and quantifies the analyte.

Tandem mass spectrometry can utilize combinations of the mass analyzers described above. Tandem mass spectrometers can use a first mass analyzer to separate ions according to their m/z in order to isolate an ion of interest for further analysis. The isolated ion of interest is then broken into fragment ions (called collisionally activated dissociation or collisionally induced dissociation) and the fragment ions are analyzed by the second mass analyzer. These types of tandem mass spectrometer systems are called tandem in space systems because the two mass analyzers are separated in space, usually by a collision cell. Tandem mass spectrometer systems also include tandem in time systems where one mass analyzer is used, however the mass analyzer is used sequentially to isolate an ion, induce fragmentation, and then perform mass analysis.

Mass spectrometers in the tandem in space category have more than one mass analyzer. For example, a tandem quadrupole mass spectrometer system can have a first quadrupole mass filter, followed by a collision cell, followed by a second quadrupole mass filter and then the detector. Another arrangement is to use a quadrupole mass filter for the first mass analyzer and a time-of-flight mass analyzer for the second mass analyzer with a collision cell separating the two mass analyzers. Other tandem systems are known in the art including reflectron-time-of-flight, tandem sector and sector-quadrupole mass spectrometry.

Mass spectrometers in the tandem in time category have one mass analyzer that performs different functions at different times. For example, an ion trap mass spectrometer can be used to trap ions of all m/z. A series of rf scan functions are applied which ejects ions of all m/z from the trap except the m/z of ions of interest. After the m/z of interest has been isolated, an rf pulse is applied to produce collisions with gas molecules in the trap to induce fragmentation of the ions. Then the m/z values of the fragmented ions are measured by the mass analyzer. Ion cyclotron resonance instruments, also known as Fourier transform mass spectrometers, are an example of tandem-in-time systems.

Several types of tandem mass spectrometry experiments can be performed by controlling the ions that are selected in each stage of the experiment. The different types of experiments utilize different modes of operation, sometimes called “scans,” of the mass analyzers. In a first example, called a mass spectrum scan, the first mass analyzer and the collision cell transmit all ions for mass analysis into the second mass analyzer. In a second example, called a product ion scan, the ions of interest are mass-selected in the first mass analyzer and then fragmented in the collision cell. The ions formed are then mass analyzed by scanning the second mass analyzer. In a third example, called a precursor ion scan, the first mass analyzer is scanned to sequentially transmit the mass analyzed ions into the collision cell for fragmentation. The second mass analyzer mass-selects the product ion of interest for transmission to the detector. Therefore, the detector signal is the result of all precursor ions that can be fragmented into a common product ion. Other experimental formats include neutral loss scans where a constant mass difference is accounted for in the mass scans.

For quantification, controls may be used which can provide a signal in relation to the amount of the nucleic acid fragment, for example, that is present or is introduced. A control to allow conversion of relative mass signals into absolute quantities can be accomplished by addition of a known quantity of a mass tag or mass label to each sample before detection of the nucleic acid fragments. Any mass tag that does not interfere with detection of the fragments can be used for normalizing the mass signal. Such standards typically have separation properties that are different from those of any of the molecular tags in the sample, and could have the same or different mass signatures.

A separation step sometimes can be used to remove salts, enzymes, or other buffer components from the nucleic acid sample. Several methods well known in the art, such as chromatography, gel electrophoresis, or precipitation, can be used to clean up the sample. For example, size exclusion chromatography or affinity chromatography can be used to remove salt from a sample. The choice of separation method can depend on the amount of a sample. For example, when small amounts of sample are available or a miniaturized apparatus is used, a micro-affinity chromatography separation step can be used. In addition, whether a separation step is desired, and the choice of separation method, can depend on the detection method used. Salts sometimes can absorb energy from the laser in matrix-assisted laser desorption/ionization and result in lower ionization efficiency. Thus, the efficiency of matrix-assisted laser desorption/ionization and electrospray ionization sometimes can be improved by removing salts from a sample.

MASSEXTEND technology may be used in some embodiments. Generally, a primer hybridizes to sample nucleic acid at a sequence within or adjacent to a site of interest. The addition of a DNA polymerase, plus a mixture of nucleotides and terminators, allows extension of the primer through the site of interest, and generates a unique mass product. The resultant mass of the primer extension product is then analyzed (e.g., using mass spectrometry) and used to determine the sequence and/or identity of the site of interest.

Nanopores

In some embodiments, nucleic acid fragments are detected and/or quantified using a nanopore. A nanopore can be used to obtain nucleotide sequencing information for nucleic acid fragments. In some embodiments, nucleic acid fragments are detected and/or quantified using a nanopore without obtaining nucleotide sequences. A nanopore is a small hole or channel, typically of the order of 1 nanometer in diameter. Certain transmembrane cellular proteins can act as nanopores (e.g., alpha-hemolysin). Nanopores can be synthesized (e.g., using a silicon platform). Immersion of a nanopore in a conducting fluid and application of a potential across it results in a slight electrical current due to conduction of ions through the nanopore. The amount of current which flows is sensitive to the size of the nanopore. As a nucleic acid fragment passes through a nanopore, the nucleic acid molecule obstructs the nanopore to a certain degree and generates a change to the current. In some embodiments, the duration of current change as the nucleic acid fragment passes through the nanopore can be measured.

In some embodiments, nanopore technology can be used in a method described herein for obtaining nucleotide sequence information for nucleic acid fragments. Nanopore sequencing is a single-molecule sequencing technology whereby a single nucleic acid molecule (e.g. DNA) is sequenced directly as it passes through a nanopore. As described above, immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree and generates characteristic changes to the current. The amount of current which can pass through the nanopore at any given moment therefore varies depending on whether the nanopore is blocked by an A, a C, a G, a T, or sometimes methyl-C. The change in the current through the nanopore as the DNA molecule passes through the nanopore represents a direct reading of the DNA sequence. In some embodiments, a nanopore can be used to identify individual DNA bases as they pass through the nanopore in the correct order (e.g., International Patent Application No. WO2010/004265).

There are a number of ways that nanopores can be used to sequence nucleic acid molecules. In some embodiments, an exonuclease enzyme, such as a deoxyribonuclease, is used. In this case, the exonuclease enzyme is used to sequentially detach nucleotides from a nucleic acid (e.g. DNA) molecule. The nucleotides are then detected and discriminated by the nanopore in order of their release, thus reading the sequence of the original strand. For such an embodiment, the exonuclease enzyme can be attached to the nanopore such that a proportion of the nucleotides released from the DNA molecule is capable of entering and interacting with the channel of the nanopore. The exonuclease can be attached to the nanopore structure at a site in close proximity to the part of the nanopore that forms the opening of the channel. In some embodiments, the exonuclease enzyme can be attached to the nanopore structure such that its nucleotide exit trajectory site is orientated towards the part of the nanopore that forms part of the opening.

In some embodiments, nanopore sequencing of nucleic acids involves the use of an enzyme that pushes or pulls the nucleic acid (e.g. DNA) molecule through the pore. In this case, the ionic current fluctuates as a nucleotide in the DNA molecule passes through the pore. The fluctuations in the current are indicative of the DNA sequence. For such an embodiment, the enzyme can be attached to the nanopore structure such that it is capable of pushing or pulling the target nucleic acid through the channel of a nanopore without interfering with the flow of ionic current through the pore. The enzyme can be attached to the nanopore structure at a site in close proximity to the part of the structure that forms part of the opening. The enzyme can be attached to the subunit, for example, such that its active site is orientated towards the part of the structure that forms part of the opening.

In some embodiments, nanopore sequencing of nucleic acids involves detection of polymerase bi-products in close proximity to a nanopore detector. In this case, nucleoside phosphates (nucleotides) are labeled so that a phosphate labeled species is released upon the addition of a polymerase to the nucleotide strand and the phosphate labeled species is detected by the pore. Typically, the phosphate species contains a specific label for each nucleotide. As nucleotides are sequentially added to the nucleic acid strand, the bi-products of the base addition are detected. The order that the phosphate labeled species are detected can be used to determine the sequence of the nucleic acid strand.

Probes

In some embodiments, nucleic acid fragments are detected and/or quantified using one or more probes. In some embodiments, quantification comprises quantifying target nucleic acid specifically hybridized to the probe. In some embodiments, quantification comprises quantifying the probe in the hybridization product. In some embodiments, quantification comprises quantifying target nucleic acid specifically hybridized to the probe and quantifying the probe in the hybridization product. In some embodiments, quantification comprises quantifying the probe after dissociating from the hybridization product. Quantification of hybridization product, probe and/or nucleic acid target can comprise use of, for example, mass spectrometry, MASSARRAY and/or MASSEXTEND technology, as described herein.

In some embodiments, probes are designed such that they each hybridize to a nucleic acid of interest in a sample. For example, a probe may comprise a polynucleotide sequence that is complementary to a nucleic acid of interest or may comprise a series of monomers that can bind to a nucleic acid of interest. Probes may be any length suitable to hybridize (e.g., completely hybridize) to one or more nucleic acid fragments of interest. For example, probes may be of any length which spans or extends beyond the length of a nucleic acid fragment to which it hybridizes. Probes may be about 10 bp or more in length. For example, probes may be at least about 20, 30, 40, 50, 60, 70, 80, 100, 200, 300, 400, 500, 600, 700, 800, 900 or 1000 bp in length. In some embodiments, a detection and/or quantification method is used to detect and/or quantify probe-nucleic acid fragment duplexes.

Probes may be designed and synthesized according to methods known in the art and described herein for oligonucleotides (e.g., capture oligonucleotides). Probes also may include any of the properties known in the art and described herein for oligonucleotides. Probes herein may be designed such that they comprise nucleotides (e.g., adenine (A), thymine (T), cytosine (C), guanine (G) and uracil (U)), modified nucleotides (e.g., mass-modified nucleotides, pseudouridine, dihydrouridine, inosine (I), and 7-methylguanosine), synthetic nucleotides, degenerate bases (e.g., 6H,8H-3,4-dihydropyrimido[4,5-c][1,2]oxazin-7-one (P), 2-amino-6-methoxyaminopurine (K), N6-methoxyadenine (Z), and hypoxanthine (I)), universal bases and/or monomers other than nucleotides, modified nucleotides or synthetic nucleotides, mass tags or combinations thereof.

In some embodiments, probes are dissociated (i.e., separated) from their corresponding nucleic acid fragments. Probes may be separated from their corresponding nucleic acid fragments using any method known in the art, including, but not limited to, heat denaturation. Probes can be distinguished from corresponding nucleic acid fragments by a method known in the art or described herein for labeling and/or isolating a species of molecule in a mixture. For example, a probe and/or nucleic acid fragment may comprise a detectable property such that a probe is distinguishable from the nucleic acid to which it hybridizes. Non-limiting examples of detectable properties include mass properties, optical properties, electrical properties, magnetic properties, chemical properties, and time and/or speed through an opening of known size. In some embodiments, probes and sample nucleic acid fragments are physically separated from each other. Separation can be accomplished, for example, using capture ligands, such as biotin or other affinity ligands, and capture agents, such as avidin, streptavidin, an antibody, or a receptor. A probe or nucleic acid fragment can contain a capture ligand having specific binding activity for a capture agent. For example, fragments from a nucleic acid sample can be biotinylated or attached to an affinity ligand using methods well known in the art and separated away from the probes using a pull-down assay with steptavidin-coated beads, for example. In some embodiments, a capture ligand and capture agent or any other moiety (e.g., mass tag) can be used to add mass to the nucleic acid fragments such that they can be excluded from the mass range of the probes detected in a mass spectrometer. In some embodiments, mass is added to the probes, addition of a mass tag for example, to shift the mass range away from the mass range for the nucleic acid fragments. In some embodiments, a detection and/or quantification method is used to detect and/or quantify dissociated nucleic acid fragments. In some embodiments, detection and/or quantification method is used to detect and/or quantify dissociated probes.

Digital PCR

In some embodiments, nucleic acid fragments are detected and/or quantified using digital PCR technology. Digital polymerase chain reaction (digital PCR or dPCR) can be used, for example, to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g. fetal alleles and maternal alleles). Alleles can be enumerated to determine copy number.

Nucleic Acid Sequencing

In some embodiments, nucleic acids (e.g., nucleic acid fragments, sample nucleic acid, circulating cell-free nucleic acid) may be sequenced. In some embodiments, a full or substantially full sequence is obtained and sometimes a partial sequence is obtained. In some embodiments, a nucleic acid is not sequenced, and the sequence of a nucleic acid is not determined by a sequencing method, when performing a method described herein. Sequencing, mapping and related analytical methods are known in the art (e.g., United States Patent Application Publication US2009/0029377, incorporated by reference). Certain aspects of such processes are described hereafter.

Certain sequencing technologies generate nucleotide sequence reads. As used herein, “reads” (i.e., “a read”, “a sequence read”) are short nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads).

In some embodiments the nominal, average, mean or absolute length of single-end reads sometimes is about 20 contiguous nucleotides to about 50 contiguous nucleotides, sometimes about 30 contiguous nucleotides to about 40 contiguous nucleotides, and sometimes about 35 contiguous nucleotides or about 36 contiguous nucleotides. In some embodiments, the nominal, average, mean or absolute length of single-end reads is about 20 to about 30 bases in length. In some embodiments, the nominal, average, mean or absolute length of single-end reads is about 24 to about 28 bases in length. In some embodiments, the nominal, average, mean or absolute length of single-end reads is about 21, 22, 23, 24, 25, 26, 27, 28 or about 29 bases in length.

In certain embodiments, the nominal, average, mean or absolute length of the paired-end reads sometimes is about 10 contiguous nucleotides to about 50 contiguous nucleotides (e.g., about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length), sometimes is about 15 contiguous nucleotides to about 25 contiguous nucleotides, and sometimes is about 17 contiguous nucleotides, about 18 contiguous nucleotides, about 20 contiguous nucleotides, about 25 contiguous nucleotides, about 36 contiguous nucleotides or about 45 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from the blood of a pregnant female can be reads from a mixture of fetal and maternal nucleic acid. A mixture of relatively short reads can be transformed by processes described herein into a representation of a genomic nucleic acid present in the pregnant female and/or in the fetus. A mixture of relatively short reads can be transformed into a representation of a copy number variation (e.g., a maternal and/or fetal copy number variation), genetic variation or an aneuploidy, for example. Reads of a mixture of maternal and fetal nucleic acid can be transformed into a representation of a composite chromosome or a segment thereof comprising features of one or both maternal and fetal chromosomes. In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

Sequence reads can be mapped and the number of reads or sequence tags mapping to a specified nucleic acid region (e.g., a chromosome, a bin, a genomic section) are referred to as counts. In some embodiments, counts can be manipulated or transformed (e.g., normalized, combined, added, filtered, selected, averaged, derived as a mean, the like, or a combination thereof). In some embodiments, counts can be transformed to produce normalized counts. Normalized counts for multiple genomic sections can be provided in a profile (e.g., a genomic profile, a chromosome profile, a profile of a segment of a chromosome). One or more different elevations in a profile also can be manipulated or transformed (e.g., counts associated with elevations can be normalized) and elevations can be adjusted.

In some embodiments, one nucleic acid sample from one individual is sequenced. In certain embodiments, nucleic acid samples from two or more biological samples, where each biological sample is from one individual or two or more individuals, are pooled and the pool is sequenced. In the latter embodiments, a nucleic acid sample from each biological sample often is identified by one or more unique identification tags.

In some embodiments, a fraction of the genome is sequenced, which sometimes is expressed in the amount of the genome covered by the determined nucleotide sequences (e.g., “fold” coverage less than 1). When a genome is sequenced with about 1-fold coverage, roughly 100% of the nucleotide sequence of the genome is represented by reads. A genome also can be sequenced with redundancy, where a given region of the genome can be covered by two or more reads or overlapping reads (e.g., “fold” coverage greater than 1). In some embodiments, a genome is sequenced with about 0.01-fold to about 100-fold coverage, about 0.2-fold to 20-fold coverage, or about 0.2-fold to about 1-fold coverage (e.g., about 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold coverage).

In certain embodiments, a subset of nucleic acid fragments is selected prior to sequencing. In certain embodiments, hybridization-based techniques (e.g., using oligonucleotide arrays) can be used to first select for nucleic acid sequences from certain chromosomes (e.g., a potentially aneuploid chromosome and other chromosome(s) not involved in the aneuploidy tested) or a segment thereof (e.g., a sub-chromosomal region). In some embodiments, nucleic acid can be fractionated by size (e.g., by gel electrophoresis, size exclusion chromatography or by microfluidics-based approach) and in certain instances, fetal nucleic acid can be enriched by selecting for nucleic acid having a lower molecular weight (e.g., less than 300 base pairs, less than 200 base pairs, less than 150 base pairs, less than 100 base pairs). In some embodiments, fetal nucleic acid can be enriched by suppressing maternal background nucleic acid, such as by the addition of formaldehyde. In some embodiments, a portion or subset of a pre-selected set of nucleic acid fragments is sequenced randomly. In some embodiments, the nucleic acid is amplified prior to sequencing. In some embodiments, a portion or subset of the nucleic acid is amplified prior to sequencing.

In some embodiments, a sequencing library is prepared prior to or during a sequencing process. Methods for preparing a sequencing library are known in the art and commercially available platforms may be used for certain applications. Certain commercially available library platforms may be compatible with certain nucleotide sequencing processes described herein. For example, one or more commercially available library platforms may be compatible with a sequencing by synthesis process. In some embodiments, a ligation-based library preparation method is used (e.g., ILLUMINA TRUSEQ, Illumina, San Diego Calif.). Ligation-based library preparation methods typically use a methylated adaptor design which can incorporate an index sequence at the initial ligation step and often can be used to prepare samples for single-read sequencing, paired-end sequencing and multiplexed sequencing. In some embodiments, a transposon-based library preparation method is used (e.g., EPICENTRE NEXTERA, Illumina, Inc., California). Transposon-based methods typically use in vitro transposition to simultaneously fragment and tag DNA in a single-tube reaction (often allowing incorporation of platform-specific tags and optional barcodes), and prepare sequencer-ready libraries.

Any sequencing method suitable for conducting methods described herein can be utilized. In some embodiments, a high-throughput sequencing method is used. High-throughput sequencing methods generally involve clonally amplified DNA templates or single DNA molecules that are sequenced in a massively parallel fashion within a flow cell (e.g. as described in Metzker M Nature Rev 11:31-46 (2010); Volkerding et al. Clin Chem 55:641-658 (2009)). Such sequencing methods also can provide digital quantitative information, where each sequence read is a countable “sequence tag” or “count” representing an individual clonal DNA template, a single DNA molecule, bin or chromosome. Next generation sequencing techniques capable of sequencing DNA in a massively parallel fashion are collectively referred to herein as “massively parallel sequencing” (MPS). Certain MPS techniques include a sequencing-by-synthesis process. High-throughput sequencing technologies include, for example, sequencing-by-synthesis with reversible dye terminators, sequencing by oligonucleotide probe ligation, pyrosequencing and real time sequencing. Non-limiting examples of MPS include Massively Parallel Signature Sequencing (MPSS), Polony sequencing, Pyrosequencing, Illumina (Solexa) sequencing, SOLiD sequencing, Ion semiconductor sequencing, DNA nanoball sequencing, Helioscope single molecule sequencing, single molecule real time (SMRT) sequencing, nanopore sequencing, ION Torrent and RNA polymerase (RNAP) sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, first generation technology, such as, for example, Sanger sequencing including the automated Sanger sequencing, can be used in a method provided herein. Additional sequencing technologies that include the use of developing nucleic acid imaging technologies (e.g. transmission electron microscopy (TEM) and atomic force microscopy (AFM)), also are contemplated herein. Examples of various sequencing technologies are described below.

A nucleic acid sequencing technology that may be used in a method described herein is sequencing-by-synthesis and reversible terminator-based sequencing (e.g. Illumina's Genome Analyzer; Genome Analyzer II; HISEQ 2000; HISEQ 2500 (IIlumina, San Diego Calif.)). With this technology, millions of nucleic acid (e.g. DNA) fragments can be sequenced in parallel. In one example of this type of sequencing technology, a flow cell is used which contains an optically transparent slide with 8 individual lanes on the surfaces of which are bound oligonucleotide anchors (e.g., adaptor primers). A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs.

In certain sequencing by synthesis procedures, for example, template DNA (e.g., circulating cell-free DNA (ccfDNA)) sometimes can be fragmented into lengths of several hundred base pairs in preparation for library generation. In some embodiments, library preparation can be performed without further fragmentation or size selection of the template DNA (e.g., ccfDNA). Sample isolation and library generation may be performed using automated methods and apparatus, in certain embodiments. Briefly, template DNA is end repaired by a fill-in reaction, exonuclease reaction or a combination of a fill-in reaction and exonuclease reaction. The resulting blunt-end repaired template DNA is extended by a single nucleotide, which is complementary to a single nucleotide overhang on the 3′ end of an adapter primer, and often increases ligation efficiency. Any complementary nucleotides can be used for the extension/overhang nucleotides (e.g., A/T, C/G), however adenine frequently is used to extend the end-repaired DNA, and thymine often is used as the 3′ end overhang nucleotide.

In certain sequencing by synthesis procedures, for example, adapter oligonucleotides are complementary to the flow-cell anchors, and sometimes are utilized to associate the modified template DNA (e.g., end-repaired and single nucleotide extended) with a solid support, such as the inside surface of a flow cell, for example. In some embodiments, the adapter also includes identifiers (i.e., indexing nucleotides, or “barcode” nucleotides (e.g., a unique sequence of nucleotides usable as an identifier to allow unambiguous identification of a sample and/or chromosome)), one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). Identifiers or nucleotides contained in an adapter often are six or more nucleotides in length, and frequently are positioned in the adaptor such that the identifier nucleotides are the first nucleotides sequenced during the sequencing reaction. In certain embodiments, identifier nucleotides are associated with a sample but are sequenced in a separate sequencing reaction to avoid compromising the quality of sequence reads. Subsequently, the reads from the identifier sequencing and the DNA template sequencing are linked together and the reads de-multiplexed. After linking and de-multiplexing the sequence reads and/or identifiers can be further adjusted or processed as described herein.

In certain sequencing by synthesis procedures, utilization of identifiers allows multiplexing of sequence reactions in a flow cell lane, thereby allowing analysis of multiple samples per flow cell lane. The number of samples that can be analyzed in a given flow cell lane often is dependent on the number of unique identifiers utilized during library preparation and/or probe design. Non limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively). A method described herein can be performed using any number of unique identifiers (e.g., 4, 8, 12, 24, 48, 96, or more). The greater the number of unique identifiers, the greater the number of samples and/or chromosomes, for example, that can be multiplexed in a single flow cell lane. Multiplexing using 12 identifiers, for example, allows simultaneous analysis of 96 samples (e.g., equal to the number of wells in a 96 well microwell plate) in an 8 lane flow cell. Similarly, multiplexing using 48 identifiers, for example, allows simultaneous analysis of 384 samples (e.g., equal to the number of wells in a 384 well microwell plate) in an 8 lane flow cell.

In certain sequencing by synthesis procedures, adapter-modified, single-stranded template DNA is added to the flow cell and immobilized by hybridization to the anchors under limiting-dilution conditions. In contrast to emulsion PCR, DNA templates are amplified in the flow cell by “bridge” amplification, which relies on captured DNA strands “arching” over and hybridizing to an adjacent anchor oligonucleotide. Multiple amplification cycles convert the single-molecule DNA template to a clonally amplified arching “cluster,” with each cluster containing approximately 1000 clonal molecules. Approximately 1×10̂9 separate clusters can be generated per flow cell. For sequencing, the clusters are denatured, and a subsequent chemical cleavage reaction and wash leave only forward strands for single-end sequencing. Sequencing of the forward strands is initiated by hybridizing a primer complementary to the adapter sequences, which is followed by addition of polymerase and a mixture of four differently colored fluorescent reversible dye terminators. The terminators are incorporated according to sequence complementarity in each strand in a clonal cluster. After incorporation, excess reagents are washed away, the clusters are optically interrogated, and the fluorescence is recorded. With successive chemical steps, the reversible dye terminators are unblocked, the fluorescent labels are cleaved and washed away, and the next sequencing cycle is performed. This iterative, sequencing-by-synthesis process sometimes requires approximately 2.5 days to generate read lengths of 36 bases. With 50×106 clusters per flow cell, the overall sequence output can be greater than 1 billion base pairs (Gb) per analytical run.

Another nucleic acid sequencing technology that may be used with a method described herein is 454 sequencing (Roche). 454 sequencing uses a large-scale parallel pyrosequencing system capable of sequencing about 400-600 megabases of DNA per run. The process typically involves two steps. In the first step, sample nucleic acid (e.g. DNA) is sometimes fractionated into smaller fragments (300-800 base pairs) and polished (made blunt at each end). Short adaptors are then ligated onto the ends of the fragments. These adaptors provide priming sequences for both amplification and sequencing of the sample-library fragments. One adaptor (Adaptor B) contains a 5′-biotin tag for immobilization of the DNA library onto streptavidin-coated beads. After nick repair, the non-biotinylated strand is released and used as a single-stranded template DNA (sstDNA) library. The sstDNA library is assessed for its quality and the optimal amount (DNA copies per bead) needed for emPCR is determined by titration. The sstDNA library is immobilized onto beads. The beads containing a library fragment carry a single sstDNA molecule. The bead-bound library is emulsified with the amplification reagents in a water-in-oil mixture. Each bead is captured within its own microreactor where PCR amplification occurs. This results in bead-immobilized, clonally amplified DNA fragments.

In the second step of 454 sequencing, single-stranded template DNA library beads are added to an incubation mix containing DNA polymerase and are layered with beads containing sulfurylase and luciferase onto a device containing pico-liter sized wells. Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing exploits the release of pyrophosphate (PPi) upon nucleotide addition. PPi is converted to ATP by ATP sulfurylase in the presence of adenosine 5′ phosphosulfate. Luciferase uses ATP to convert luciferin to oxyluciferin, and this reaction generates light that is discerned and analyzed (see, for example, Margulies, M. et al. Nature 437:376-380 (2005)).

Another nucleic acid sequencing technology that may be used in a method provided herein is Applied Biosystems' SOLiD™ technology. In SOLiD™ sequencing-by-ligation, a library of nucleic acid fragments is prepared from the sample and is used to prepare clonal bead populations. With this method, one species of nucleic acid fragment will be present on the surface of each bead (e.g. magnetic bead). Sample nucleic acid (e.g. genomic DNA) is sheared into fragments, and adaptors are subsequently attached to the 5′ and 3′ ends of the fragments to generate a fragment library. The adapters are typically universal adapter sequences so that the starting sequence of every fragment is both known and identical. Emulsion PCR takes place in microreactors containing all the necessary reagents for PCR. The resulting PCR products attached to the beads are then covalently bound to a glass slide. Primers then hybridize to the adapter sequence within the library template. A set of four fluorescently labeled di-base probes compete for ligation to the sequencing primer. Specificity of the di-base probe is achieved by interrogating every 1st and 2nd base in each ligation reaction. Multiple cycles of ligation, detection and cleavage are performed with the number of cycles determining the eventual read length. Following a series of ligation cycles, the extension product is removed and the template is reset with a primer complementary to the n-1 position for a second round of ligation cycles. Often, five rounds of primer reset are completed for each sequence tag. Through the primer reset process, each base is interrogated in two independent ligation reactions by two different primers. For example, the base at read position 5 is assayed by primer number 2 in ligation cycle 2 and by primer number 3 in ligation cycle 1.

Another nucleic acid sequencing technology that may be used in a method described herein is Helicos True Single Molecule Sequencing (tSMS). In the tSMS technique, a polyA sequence is added to the 3′ end of each nucleic acid (e.g. DNA) strand from the sample. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm2. The flow cell is then loaded into a sequencing apparatus and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step (see, for example, Harris T. D. et al., Science 320:106-109 (2008)).

Another nucleic acid sequencing technology that may be used in a method provided herein is the single molecule, real-time (SMRT™) sequencing technology of Pacific Biosciences. With this method, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is then repeated.

Another nucleic acid sequencing technology that may be used in a method described herein is ION TORRENT (Life Technologies) single molecule sequencing which pairs semiconductor technology with a simple sequencing chemistry to directly translate chemically encoded information (A, C, G, T) into digital information (0, 1) on a semiconductor chip. ION TORRENT uses a high-density array of micro-machined wells to perform nucleic acid sequencing in a massively parallel way. Each well holds a different DNA molecule. Beneath the wells is an ion-sensitive layer and beneath that an ion sensor. Typically, when a nucleotide is incorporated into a strand of DNA by a polymerase, a hydrogen ion is released as a byproduct. If a nucleotide, for example a C, is added to a DNA template and is then incorporated into a strand of DNA, a hydrogen ion will be released. The charge from that ion will change the pH of the solution, which can be detected by an ion sensor. A sequencer can call the base, going directly from chemical information to digital information. The sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection (i.e. detection without scanning, cameras or light), each nucleotide incorporation is recorded in seconds.

Another nucleic acid sequencing technology that may be used in a method described herein is the chemical-sensitive field effect transistor (CHEMFET) array. In one example of this sequencing technique, DNA molecules are placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a CHEMFET sensor. An array can have multiple CHEMFET sensors. In another example, single nucleic acids are attached to beads, and the nucleic acids can be amplified on the bead, and the individual beads can be transferred to individual reaction chambers on a CHEMFET array, with each chamber having a CHEMFET sensor, and the nucleic acids can be sequenced (see, for example, U.S. Patent Application Publication No. 2009/0026082).

Another nucleic acid sequencing technology that may be used in a method described herein is electron microscopy. In one example of this sequencing technique, individual nucleic acid (e.g. DNA) molecules are labeled using metallic labels that are distinguishable using an electron microscope. These molecules are then stretched on a flat surface and imaged using an electron microscope to measure sequences (see, for example, Moudrianakis E. N. and Beer M. Proc Natl Acad Sci USA. 1965 March; 53:564-71). In some embodiments, transmission electron microscopy (TEM) is used (e.g. Halcyon Molecular's TEM method). This method, termed Individual Molecule Placement Rapid Nano Transfer (IMPRNT), includes utilizing single atom resolution transmission electron microscope imaging of high-molecular weight (e.g. about 150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA (see, for example, International Patent Application No. WO 2009/046445).

Other sequencing methods that may be used to conduct methods herein include digital PCR and sequencing by hybridization. Digital polymerase chain reaction (digital PCR or dPCR) can be used to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g. fetal alleles and maternal alleles). Alleles can be enumerated to determine copy number. In sequencing by hybridization, the method involves contacting a plurality of polynucleotide sequences with a plurality of polynucleotide probes, where each of the plurality of polynucleotide probes can be optionally tethered to a substrate. The substrate can be a flat surface with an array of known nucleotide sequences, in some embodiments. The pattern of hybridization to the array can be used to determine the polynucleotide sequences present in the sample. In some embodiments, each probe is tethered to a bead, e.g., a magnetic bead or the like. Hybridization to the beads can be identified and used to identify the plurality of polynucleotide sequences within the sample.

In some embodiments, chromosome-specific sequencing is performed. In some embodiments, chromosome-specific sequencing is performed utilizing DANSR (digital analysis of selected regions). Digital analysis of selected regions enables simultaneous quantification of hundreds of loci by cfDNA-dependent catenation of two locus-specific oligonucleotides via an intervening ‘bridge’ oligo to form a PCR template. In some embodiments, chromosome-specific sequencing is performed by generating a library enriched in chromosome-specific sequences. In some embodiments, sequence reads are obtained only for a selected set of chromosomes.

The length of the sequence read often is associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median, mode or average length of about 4 bp to 900 bp long (e.g. about 5 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median, mode or average length of about 1,000 bp or more.

### Genotype Uses

A genotype for a subject may be provided for the purpose of predicting an ocular VEGF suppression response and required dosing interval to a treatment that suppresses ocular VEGF. An ocular VEGF suppression response prediction sometimes is provided by an entity that provides the genotype. An ocular VEGF suppression response prediction sometimes is provided to a patient (i.e., test subject) by a health care provider who utilizes the genotype for providing the prediction. A health care provider may base the prediction solely on a genotype for a subject, or may utilize the genotype in conjunction with other information to provide the prediction (i.e., collectively providing a prediction according to the genotype). Other information that a health care provider may utilize to provide a prediction includes smoking history, age, BMI and other information known as being associated with an ocular degeneration condition (e.g., described herein), for example.

An ocular VEGF suppression response predicted sometimes is an ocular VEGF suppression time in response to a VEGF suppressor. An ocular suppression time prediction for a subject sometimes is provided in units of days.

An ocular VEGF suppression time sometimes is provided with or without an estimate of variation or error. An estimate of variation or error can be expressed using one or more suitable statistics known in the art. An estimate of variation or error sometimes is an indicator for accuracy (e.g., standard error) or precision (e.g., coefficient of variation), or accuracy and precision, of a predicted ocular VEGF suppression time. Non-limiting examples of estimates of variation or error include standard error, relative standard error, normalized standard error, standard error of the mean (SEM), standard deviation, relative standard deviation, coefficient of variation, root mean square deviation (RMSD), root mean square error (RMSE), normalized root mean square deviation (NRMSD), normalized root mean square error (NRMSE), coefficient of variation of the root mean square deviation (CVRMSD) and coefficient of variation of the root mean square error (CVRMSE). An estimate of error sometimes is about 15% of the ocular VEGF suppression time prediction, or less (e.g., about 14% of the ocular VEGF suppression time prediction or less, 13% of the ocular VEGF suppression time prediction or less, 12% of the ocular VEGF suppression time prediction or less, 11% of the ocular VEGF suppression time prediction or less, 10% of the ocular VEGF suppression time prediction or less, 9% of the ocular VEGF suppression time prediction or less, 8% of the ocular VEGF suppression time prediction or less, 7% of the ocular VEGF suppression time prediction or less, 6% of the ocular VEGF suppression time prediction or less, 5% of the ocular VEGF suppression time prediction or less, 4% of the ocular VEGF suppression time prediction or less, 3% of the ocular VEGF suppression time prediction or less, 1% of the ocular VEGF suppression time prediction or less, 1% of the ocular VEGF suppression time prediction or less). An estimate of variation or error can be utilized to determine a confidence level for the prediction, such as a confidence interval (e.g., 95% confidence interval, 90% confidence interval), for example.

An ocular VEGF suppression response sometimes is categorization of a subject into an ocular VEGF suppression time group. Non-limiting examples of such groups are subjects displaying a relatively low ocular VEGF suppression time, subjects displaying a relatively high ocular VEGF suppression time and subjects displaying a relatively average ocular VEGF suppression time (e.g., mean, median, mode). A relatively short ocular VEGF suppression time sometimes is at least about 5 days less (e.g., about 15, 14, 13, 12, 11, 10, 9, 8, 7, 6 days less) than the average VEGF suppression time (e.g., mean, median, mode) for a population in response to a particular VEGF suppressor. A relatively long VEGF ocular suppression time sometimes is at least about 5 days more (e.g., about 6, 7, 8, 9, 10, 11, 12, 13, 14, 15 days more) than the average VEGF suppression time (e.g., mean, median, mode) for a population in response to a particular VEGF suppressor. A relatively average ocular VEGF suppression time sometimes is within about 5 days (e.g., about 4, 3, 2, 1 days) of the average VEGF suppression time (e.g., mean, median, mode) for a population in response to a particular VEGF suppressor.

Confidence associated with categorizing a test subject into a ocular VEGF suppression response group (e.g., ocular VEGF suppression time group) can be assessed with any suitable statistical method known in the art, and can be provided as any suitable statistic known in the art. Non-limiting examples of such statistics include sensitivity (e.g., a sensitivity of 0.80 or greater (e.g., 0.85, 0.90, 0.91, 0.92, 0.93, 0.94, 0.95, 0.96, 0.97, 0.98, 0.99)), specificity (e.g., a specificity of 0.80 or greater (e.g., 0.85, 0.90, 0.91, 0.92, 0.93, 0.94, 0.95, 0.96, 0.97, 0.98, 0.99)), area under the curve (AUC) for a receiver operating characteristic (ROC) curve (e.g., an AUC of 0.70 or greater (e.g., 0.75, 0.80, 0.85, 0.90, 0.91, 0.92, 0.93, 0.94, 0.95, 0.96, 0.97, 0.98, 0.99)), or a combination of the foregoing.

In certain embodiments, a genotype comprising two alleles of rs1870377 is determined, and a VEGF suppression time predicted for a genotype comprising homozygous thymine alleles is longer than a VEGF suppression time predicted for a genotype comprising heterozygous adenine and thymine alleles. In some embodiments, a genotype comprising two alleles of rs1870377 is determined, and a relatively high VEGF suppression time is predicted for a genotype comprising homozygous thymine alleles. In certain embodiments, a genotype comprising two alleles of rs2071559 is determined, and a VEGF suppression time predicted for a genotype comprising heterozygous guanine and adenine alleles is longer than (i) a VEGF suppression time predicted for a genotype comprising homozygous adenine alleles, and (ii) a VEGF suppression time predicted for a genotype comprising homozygous guanine alleles. In some embodiments, a genotype comprising two alleles of rs1870377 and two alleles of rs2071559 is determined, and (i) a VEGF suppression time predicted for a genotype comprising heterozygous guanine and adenine alleles for rs2071559 and homozygous thymine alleles for rs1870377, is longer than (ii) a VEGF suppression time predicted for a genotype comprising homozygous guanine or adenine alleles for rs2071559 and homozygous adenine alleles or heterozygous adenine and thymine alleles for rs1870377. In certain embodiments, a genotype comprising two alleles of rs1870377 and two alleles of rs2071559 is determined, and a relatively long VEGF suppression time is predicted for a genotype comprising heterozygous guanine and adenine alleles for rs2071559 and homozygous thymine alleles for rs1870377. In some embodiments, a genotype comprising two alleles of rs1870377 and two alleles of rs2071559 is determined, and a relatively short VEGF suppression time is predicted for a genotype comprising homozygous guanine or adenine alleles for rs2071559 and homozygous adenine alleles or heterozygous adenine and thymine alleles for rs1870377.

A genotype sometimes is utilized to select and/or dose a VEGF suppressor agent for a subject or group of subjects. A VEGF suppressor agent sometimes is an ocular VEGF suppressor agent, and an ocular VEGF suppression response prediction sometimes is utilized to select and/or dose the agent for a particular subject or group of subjects.

A genotype and/or ocular VEGF suppression response prediction sometimes is utilized to select a dosing interval for a subject for a particular VEGF suppressor. An ocular VEGF suppression time sometimes is predicted according to a genotype for a subject, and a dosing interval for a particular VEGF suppressor is selected according to the ocular VEGF suppression time prediction. The dosing interval selected sometimes is less than or equal to the ocular VEGF suppression time predicted for the subject. A dosing interval sometimes is about 5 days or less (e.g., about 4, 3, 2, 1 day(s) less) than the ocular VEGF suppression time predicted for the subject.

A genotype and/or ocular VEGF suppression response prediction sometimes is utilized to select a VEGF suppression treatment for administration to a subject. A VEGF suppression treatment sometimes is selected according to an average VEGF suppression time (e.g., average ocular VEGF suppression time) for the treatment in a population. An average VEGF suppression time for the treatment often is inversely proportional to, or greater than, an ocular VEGF suppression time prediction for a subject. A treatment sometimes is selected for which an average ocular VEGF suppression time is (i) fewer than 5 days (e.g., 4, 3, 2, 1 day(s)) less than, or (ii) at least one day (e.g., 2, 3, 4, 5, 6, 7, 8, 9, 10, 11, 12, 13, 14, 15 days) greater than, an ocular VEGF suppression time predicted for a subject.

In some embodiments, a genotype comprising two alleles of rs1870377 and two alleles of rs2071559 is determined, and a treatment predicted to suppress VEGF for a relatively shorter amount of time is selected for a genotype comprising heterozygous guanine and adenine alleles for rs2071559 and homozygous thymine alleles for rs1870377. In certain embodiments, genotype comprising two alleles of rs1870377 and two alleles of rs2071559 is determined, and a treatment predicted to suppress VEGF for a relatively longer amount of time is selected for a genotype comprising homozygous guanine or adenine alleles for rs2071559 and homozygous adenine alleles or heterozygous adenine and thymine alleles for rs1870377.

A genotype and/or ocular VEGF suppression response prediction sometimes is utilized to select a VEGF suppression treatment for administration to a subject according to potency of a VEGF suppressor. In some embodiments, potency of the treatment is inversely proportional to the suppression time prediction for the subject. For example, a subject for whom a relatively low ocular VEGF response time is predicted may be administered a relatively more potent VEGF suppressor, such as aflibercept (relative to ranibizumab or bevacizumab).

In another example, a subject for whom a relatively long ocular VEGF response time is predicted may be administered a relatively less potent VEGF suppressor, such as pegaptanib (relative to ranibizumab or bevacizumab).

### Macular Degeneration Disorder Treatments

Any suitable type of macular degeneration treatment may be administered to a subject for whom a genotype has been obtained. A macular degeneration treatment selected often suppresses ocular VEGF in a subject for a period of time.

A treatment selected sometimes inhibits association of a VEGF to a native VEGF receptor (VEGFR). The VEGF and/or VEGFR targeted by the treatment generally is/are present in the eye. A treatment selected sometimes comprises an agent configured to specifically associate with a VEGF (e.g., specifically bind to a VEGF present in the eye), specifically cleave a VEGF, specifically inhibit production of a VEGF, or combination of the foregoing. A treatment selected sometimes comprises an agent configured to specifically associate with a VEGFR (e.g., specifically bind to a VEGFR present in the eye), specifically cleave a VEGFR, specifically inhibit production of a VEGFR, or combination of the foregoing. Non-limiting examples of agents configured to specifically cleave a VEGFR are pigment epithelium-derived factor (PEDF), which also is known as serpin F1 (SERPINF1), and small molecule brivanib. Non-limiting examples of agents configured to specifically inhibit production of a VEGF or VEGFR include agents that inhibit production of a VEGF or VEGFR mRNA (e.g., transcription factor inhibitor, splice mechanism inhibitor, RNAi, siRNA, catalytic RNA).

A treatment selected sometimes comprises an agent configured to inhibit intracellular signaling of a VEGFR. A treatment selected sometimes comprises an agent configured to inhibit a protein tyrosine kinase involved in a VEGFR signaling pathway. A therapeutic agent sometimes inhibits (e.g., specifically inhibits) an intracellular protein tyrosine kinase, and sometimes inhibits a receptor protein tyrosine kinase (RTK). A therapeutic agent sometimes is a multi-targeted protein tyrosine kinase inhibitor, non-limiting examples of which include sunitinib, sorafenib, pazopanib and vatalanib.

A VEGF targeted by a treatment sometimes is a VEGF-A, VEGF-B, VEGF-C, VEGF-D, VEGF-E, a splice variant of any one of the foregoing, subtype of any one of the foregoing, or a combination of at least two of the foregoing. An agent that targets a VEGF sometimes associates with (e.g., binds to) placental growth factor (PIGF), or portion thereof that includes a structure similar to VEGF. Accordingly, a therapeutic agent may be selected that can associate with, cleave, or inhibit production of a VEGF nucleic acid or protein, or another molecule having a structure similar to a structure in a VEGF nucleic acid or protein (e.g., PIGF).

A VEGFR targeted by a treatment sometimes is a VEGFR-1(FLT1), VEGFR-2(FLK/KDR), VEGFR-3(FLT4), splice variant of any one of the foregoing, subtype of any one of the foregoing, or combination of any two of the foregoing. An agent that targets a VEGFR sometimes associates with (e.g., binds to) neuroplilin 1 (NRP1), neuropilin 2 (NRP2), or portion thereof that includes a structure similar to VEGFR. Accordingly, a therapeutic agent may be selected that can associate with, cleave, inhibit production of, or inhibit signaling of a VEGFR nucleic acid or protein or another molecule having a structure similar to a structure in VEGFR nucleic acid or protein (e.g., neuropilin).

A therapeutic selected sometimes includes an antibody agent or functional fragment thereof. Non-limiting examples of antibody agents are ranibizumab, bevacizumab or a functional fragment of one of the foregoing antibodies (ranibizumab and bevacizumab specifically bind to certain VEGF-A subtypes); an antibody or functional fragment that specifically binds to VEGFR-2 (e.g., DC101 antibody); and an antibody or functional fragment thereof that specifically binds to a neuropilin protein.

A therapeutic selected sometimes includes an ankyrin repeat protein agent or functional fragment thereof. An ankyrin repeat protein sometimes is referred to as a DARPin, and a non-limiting example of an ankyrin repeat protein is MP0112, which specifically binds to certain VEGF-A subtypes.

A therapeutic selected sometimes includes an aptamer nucleic acid agent or functional fragment thereof. Non-limiting examples of aptamers include pegaptanib (binds to VEGF165); V7t1 (binds to VEGF165 and VEGF121 (VEGF-A subtypes)) and a combination thereof.

A therapeutic selected sometimes includes a soluble VEGFR agent or functional fragment thereof. Such agents can function as VEGF decoys or VEGF traps, sometimes are endogenous receptor (e.g., sFLT01) or a functional fragment thereof, and sometimes are recombinant receptor or a functional fragment thereof. Such agents sometimes are fusion proteins that can include any suitable number of VEGFR agents or functional fragments thereof, and can optionally include an antibody or antibody fragment (e.g., Fc fragment. A fusion protein sometimes includes immunoadhesin function, and can often specifically bind to one or more molecules to which it is targeted. A fusion protein can include one or more VEGF-binding portions from extracellular domains of human VEGFR-1 and VEGFR-2 fused to the Fc portion of the human IgG1 immunoglobulin (e.g., aflibercept). A fusion protein can include, for example, the second Ig domain of VEGFR1 and the third and fourth Ig domain of VEGFR2 fused to the constant region (Fc) of human IgG1 (e.g., conbercept).

A therapeutic selected sometimes includes a non-signal transducing VEGFR ligand. A non-signal transducing VEGFR ligand sometimes is native or recombinant, and sometimes is full length or a functional fragment thereof or a synthetic analog. Non-limiting examples of such agents include VEGF 120/121b, VEGF164b/165b, VEGF188b/189b molecule, or functional fragment or synthetic analog thereof.

In certain embodiments, a treatment selected includes administration of an agent chosen from ranibizumab, bevacizumab, aflibercept and pegaptanib. In some embodiments, a treatment selected includes administration of a photodynamic therapy (PDT), a photocoagulation therapy, or stereotactic radiosurgery, epimacular brachytherapy, or combination of any two or more of the foregoing.

### EXAMPLES

The examples set forth below illustrate certain embodiments and do not limit the technology.

### Example 1

### Sample Collection and Measurements

In this study with a follow-up of 12 months we included 283 patients from two study centers. Initial treatment consisted in 3 monthly ranibizumab injections. On monthly follow-up visits additional series of 3 monthly ranibizumab injections were initiated if necessary on the basis of clinical retreatment criteria. Multivariate data analysis was used to determine the influence of 125 selected tagged single nucleotide polymorphisms (tSNPs) in the VEGFA gene on visual acuity (VA) outcome at 12 months.

Patients were recruited for a prospective cohort study and informed written consent was obtained from all patients. The protocol was approved by the Ethics Committee of the University of Cologne and followed the tenets of the Declaration of Helsinki.

The patients included had active sub- or juxtafoveal CNV due to AMD confirmed by spectral-domain (SD) OCT and fluorescein angiography (FA) with indocyanine green. Further criteria in the study eye were no previous treatment for exudative AMD, such as photodynamic therapy or intravitreal injections in the study eye. Exclusion criteria included any previous ophthalmic surgery, except for cataract removal.

Patients initially received 3 consecutive, monthly intravitreal injections of 0.5 mg ranibizumab and were followed monthly for further evaluation and potential re-treatment. The consequent varying intervals between injections helped to determine the suppression duration of VEGF.

Before all intravitreal injections, 0.1 ml of aqueous humor was collected via a limbal paracentesis with a 30-gauge needle connected to an insulin syringe and immediately stored at −80° C. in sterile polypropylene tubes until analysis. Aqueous humor samples were analyzed with the Luminex xMAP microbead multiplex technology (Luminex 200, Luminex Inc., Austin, Tex.). Undiluted samples (50 μl) were analyzed and incubated for 2 hours at room temperature, protected from light. Analyses were performed according to the manufacturer's instructions (Angiogenesis Panel; R&D Systems, Wiesbaden, Germany). Standard curves for VEGF were generated using the reference standard supplied with the kit and showed a detection threshold of 4 pg/ml for VEGF.

### Example 2

### Genetic Analysis of SNPs and VEGF Suppression Time

Genetic analysis was performed for the suppression time of the VEGF treatment drug ranibizumab. Multiple SNP positions were genotyped using a multiplexed mass spectrometry extension assay (see, e.g., Oeth P, del Mistro G, Marnellos G, Shi T, van den Boom D, Qualitative and quantitative genotyping using single base primer extension coupled with matrix-assisted laser desorption/ionization time-of-flight mass spectrometry (MassARRAY), Methods Mol. Biol. 578:307-43 (2009) doi: 10.1007/978-1-60327-411-1_{—}20). Table B hereafter provides polymerase chain reaction (PCR) primers and extension oligonucleotides utilized for genotyping particular SNP positions.

TABLE B
PCR primers and extension primers

| | | | Extension |
|---|---|---|---|
| SNP rsID | PCR primer 2 | PCR primer 1 | Oligonucleotide |
| rs3025033 | ACGTTGGATGTTAGGGAAGTCCTTGGAGTG | ACGTTGGATGGTTTCACATAGGGCCAAGAC | CTCCCCTCCCCCAGC |
| rs3025039 | ACGTTGGATGAGACTCCGGCGGAAGCATT | ACGTTGGATGAACTCTCTAATCTTCCGGGC | aGGGCGGGTGACCCAGCA |
| rs2071559 | ACGTTGGATGGGAGCACGATGGACAAAAGC | ACGTTGGATGATCAGAAAACGCACTTGCCC | TTGGGAAATAGCGGGAATG |
| rs1870377 | ACGTTGGATGTCCTCCACACTTCTCCATTC | ACGTTGGATGCTTTTCCTTACTCTTGACTC | gggcTTGTCACTGAGACAGC |
| rs2305948 | ACGTTGGATGGAAACTTGTAAACCGAGACC | ACGTTGGATGGTACAATCCTTGGTCACTCC | AGCACCTTAACTATAGATGGT |

Statistics were performed using the statistical package ‘R’, version 2.15.2. A total of 426 samples were genotyped at 46 SNP locations, with a net call rate of 96%. Missing values were imputed using a k-nearest neighbor (KNN) analysis (k=10) with SNP values coded as 0,1,2 based on allele frequency (homozygous major, heterozygous, homozygous minor). Complement Factor H (CFH) haplotype states were determined from the SNPs rs1061170, rs12144939, and rs2274700 (Table 1). Lifetime risk scores were determined using the RetnaGene V1 formula.

TABLE 1
CFH Haplotype Calls

| rs1061170 | rs12144939 | rs2274700 | Haplotype |
|---|---|---|---|
| CC | GG | CC | H1/H1 |
| CT | GG | CT | H1/H2 |
| CT | GG | CC | H1/H3 |
| CT | GT | CT | H1/H4 |
| TT | GG | TT | H2/H2 |
| TT | GG | CT | H2/H3 |
| TT | GT | TT | H2/H4 |
| TT | GG | CC | H3/H3 |
| TT | GT | CT | H3/H4 |
| TT | TT | TT | H4/H4 |

Ten (10) SNP markers with allele frequencies less than 10% were dropped. Samples with VEGF suppression times (N=44) were tested for association with environmental and genetic factors using linear regression and F-statistics (Table 2). SNPs were tested using an indicator variable for each combination of alleles.

TABLE 2
Variable models and association p-values

| Variable | Model | Pvalue |
|---|---|---|
| rs2071559 | GG = 33.27, GA = 38.94, | 0.00047 |
| | AA = 33 | |
| rs1870377 | TT = 38.33, AT = 33.3, | 0.0057 |
| | AA = 35.67 | |
| rs3025033 | AA = 34.29, AG = 38, | 0.023 |
| | GG = 44 | |
| rs3025039 | CC = 34.5, CT = 37.73, | 0.047 |
| | TT = 44 | |
| rs2010963 | GG = 37, CG = 35.16, | 0.16 |
| | CC = 32.71 | |
| rs833068 | GG = 36.89, GA = 35.17, | 0.18 |
| | AA = 32.71 | |
| rs833069 | TT = 36.89, TC = 35.17, | 0.18 |
| | CC = 32.71 | |
| rs735286 | CC = 36.89, CT = 35.17, | 0.18 |
| | TT = 32.71 | |
| rs3024997 | GG = 36.89, GA = 35.17, | 0.18 |
| | AA = 32.71 | |
| Age_first_{—} | Intercept = 23.71, | 0.21 |
| ranibizumab | Age_first_ranibizumab = 0.15 | |
| rs2230199 | CC = 34.65, CG = 35.89, | 0.23 |
| | GG = 40 | |
| rs12264 | TT = 36.9, TC = 34, | 0.23 |
| | CC = 34.86 | |
| rs699946 | AA = 36.54, AG = 34.69, | 0.29 |
| | GG = 32.75 | |
| smoking | current = 33.33, stopped = 35.75, | 0.3 |
| | never = 36.69 | |
| rs7692791 | TT = 33.79, CT = 36.29, | 0.32 |
| | CC = 36.44 | |
| rs10020464 | CC = 35.44, CT = 36.08, | 0.33 |
| | TT = 31.33 | |
| BMI | Intercept = 40.77, BMI = −0.19 | 0.41 |
| rs10490924 | GG = 34.25, GT = 36.38, | 0.47 |
| | TT = 36.08 | |
| rs1061147 | AA = 35.13, CA = 35.14, | 0.59 |
| | CC = 37.25 | |
| rs1061170 | CC = 35.13, TC = 35.14, | 0.59 |
| | TT = 37.25 | |
| rs3025021 | CC = 36.53, CT = 34.82, | 0.59 |
| | TT = 35.2 | |
| gender | male = 35.1, female = 35.91 | 0.61 |
| rs699947 | CC = 34.79, CA = 35.52, | 0.66 |
| | AA = 37 | |
| rs35569394 | DEL = 34.79, DEL.INS = 35.52, | 0.66 |
| | INS = 37 | |
| rs1005230 | CC = 34.79, CT = 35.52, | 0.66 |
| | TT = 37 | |
| rs833061 | TT = 34.79, TC = 35.52, | 0.66 |
| | CC = 37 | |
| rs1413711 | CC = 34.77, CT = 35.5, | 0.66 |
| | TT = 37 | |
| rs2305948 | CC = 35.74, TC = 35 | 0.67 |
| rs2146323 | CC = 35.5, CA = 35.89, | 0.74 |
| | AA = 33.33 | |
| rs13207351 | AA = 36, GA = 35.59, | 0.77 |
| | GG = 34.29 | |
| rs2274700 | CC = 35.11, CT = 36.1 7, | 0.78 |
| | TT = 36.5 | |
| rs403846 | AA = 35.61, AG = 35, | 0.82 |
| | GG = 36.33 | |
| rs2235611 | CC = 35.44, TC = 35.75 | 0.86 |
| rs12144939 | GG = 35.64, GT = 34.71, | 0.88 |
| | TT = 37 | |
| rs1409153 | GG = 35.82, GA = 35.06, | 0.9 |
| | AA = 35.8 | |
| rs1570360 | GG = 35.67, GA = 35.42, | 0.95 |
| | AA = 34 | |
| CFHHaplotype | H1/H1 = 35.13, H1/H2 = 37.17, | 0.96 |
| | H1/H3 = 34.55, | |
| | H1/H4 = 33.75, H2/H2 = 36, | |
| | H2/H3 = 41, | |
| | H2/H4 = 36.5, H3/H4 = 35, | |
| | H4/H4 = 37 | |
| rs10922153 | GG = 35.61, GT = 35.61, | 0.97 |
| | TT = 35.12 | |
| rs1750311 | CC = 35.6, CA = 35.5, | 0.98 |
| | AA = 35 | |
| rs698859 | GG = 35.31, AG = 35.62, | 0.98 |
| | AA = 35.6 | |
| V1LTR | Intercept = 35.56, | 0.99 |
| | V1LTR = −0.05 | |
| rs2990510 | TT = 35.57, GT = 35.44, | 1 |
| | GG = 35.6 | |

One SNP, rs2071559, was shown to have a statistically significant association, passing a Bonferroni correction threshold (Table 2). A second SNP, rs1870377, showed a promising association (p<0.01) and two others, rs3025033 and rs3025039, showed a potential weak association (p<0.05). Interaction tests were performed for these SNPs and showed no significant effects (p>0.05).

The underlying SNP models (ex. dominant, recessive) for the top two associations, rs2071559 and rs1870377, were determined from visual inspections of the VEGF suppression time data. Together they form a two-SNP model, which predicts a longer response time for rs2071559 T-homozygous individuals and a longer response time for rs2071559 AG-heterozygous individuals. The two-SNP model coefficients were trained using linear regression of the two SNP indicator variables. Estimated VEGF response times for each sample group are shown in Table 3.

TABLE 3
2-SNP model estimates for VEGF suppression time (days)

| | rs2071559 (TT) | rs2071559 (AA/AT) |
|---|---|---|
| rs2071559 (AG) | 40.9 [38.28-43.42] | 36.96 [34.88-38.98] |
| rs2071559 (AA/GG) | 35.74 [34.15-37.43] | 31.8 [30.11-33.54] |

Confidence intervals (95%) in Table 3 were estimated using bootstrapping. The standard error (root mean square error (RMSE)) for VEGF suppression time was 3.8 days using this the-SNP model, compared to a standard deviation of 5.1 days when using the mean suppression time.

Diagnostic metrics using the two-SNP model were assessed for a two-category test. The two categories were (i) relatively short VEGF suppression time (<=35 days), and (ii) relatively high VEGF suppression time (>35 days). Assay sensitivity and specificity were estimated with a receiver operating characteristic (ROC) curve. The estimated area under the curve (AUC) was 0.73. This AUC value is evidence that these two markers are useful for predicting whether a subject will respond to an anti-VEGF agent with a relatively short VEGF suppression time or a relatively long VEGF suppression time. As there was not an independent test cohort, standard error and ROC statistics were based on the training set. This approach likely resulted in an inflated AUC and a reduced RMSE to some degree.

### Example 3

### Linkage Disequilibrium Analysis of Genetic Variants

Provided in the following table are R-squared and D-prime assessments of genetic markers in linkage disequilibrium with certain query SNP markers (left-most column). These assessments were provided using a SNP Annotation and Proxy (SNAP) search (Broad Institute).

TABLE 4
Linkage disequilibrium analysis of SNP variants

| SNP | Proxy | Distance | RSquared | DPrime | Arrays | Chromosome | Coordinate_HG18 |
|---|---|---|---|---|---|---|---|
| rs1870377 | rs1870377 | 0 | 1 | 1 | AG, I1, IM, IMD, IBC, OQ, OE, O24, | chr4 | 55667731 |
| | | | | | O28, O54, O5E, OEE, AAE | | |
| rs1870377 | rs7677779 | 5290 | 0.959 | 1 | None | chr4 | 55662441 |
| rs1870377 | rs13136007 | 3968 | 0.92 | 1 | IBC | chr4 | 55663763 |
| rs1870377 | rs58415820 | 7540 | 0.916 | 0.957 | None | chr4 | 55660191 |
| rs1870377 | rs2305946 | 8369 | 0.916 | 0.957 | None | chr4 | 55659362 |
| rs1870377 | rs3816584 | 8409 | 0.916 | 0.957 | AAH | chr4 | 55659322 |
| rs1870377 | rs6838752 | 8873 | 0.916 | 0.957 | I2, I5, I6, I6Q, IM, IMD, OQ, IWQ, OE, O24, | chr4 | 55658858 |
| | | | | | O28, O54, O5E, OEE | | |
| rs1870377 | rs2219471 | 11815 | 0.916 | 0.957 | AS, A5, A6, I3, I5, I6, I6Q, IM, IMD, IC, ICQ, CYT, OQ, | chr4 | 55655916 |
| | | | | | IWQ, OE, O24, O28, O54, O5E, OEE, AAH | | |
| rs1870377 | rs1870378 | 6521 | 0.876 | 0.956 | I1, IM, IMD, O54, O5E | chr4 | 55661210 |
| rs1870377 | rs1870379 | 6670 | 0.876 | 0.956 | None | chr4 | 55661061 |
| rs1870377 | rs35624269 | 12879 | 0.876 | 0.956 | None | chr4 | 55654852 |
| rs1870377 | rs17085267 | 13112 | 0.876 | 0.956 | None | chr4 | 55654619 |
| rs1870377 | rs17085265 | 13822 | 0.876 | 0.956 | OQ | chr4 | 55653909 |
| rs1870377 | rs17085262 | 13833 | 0.876 | 0.956 | IBC, AxM | chr4 | 55653898 |
| rs1870377 | rs13127286 | 15658 | 0.876 | 0.956 | None | chr4 | 55652073 |
| rs1870377 | rs10016064 | 2769 | 0.834 | 0.913 | None | chr4 | 55664962 |
| rs1870377 | rs4864532 | 12570 | 0.674 | 0.858 | None | chr4 | 55655161 |
| rs1870377 | rs1458830 | 14972 | 0.447 | 0.937 | None | chr4 | 55652759 |
| rs1870377 | rs17709898 | 15251 | 0.447 | 0.937 | I2, I5, I6, I6Q, IM, IMD, OQ, IWQ, OE, O24, | chr4 | 55652480 |
| | | | | | O28, O54, O5E, OEE | | |
| rs1870377 | rs11940163 | 15659 | 0.447 | 0.937 | None | chr4 | 55652072 |
| rs1870377 | rs7671745 | 16138 | 0.447 | 0.937 | AxM | chr4 | 55651593 |
| rs1870377 | rs6846151 | 1316 | 0.329 | 1 | None | chr4 | 55669047 |
| rs1870377 | rs17085326 | 4402 | 0.316 | 0.897 | IBC, OQ, AxM | chr4 | 55672133 |
| rs1870377 | rs7673274 | 2745 | 0.308 | 1 | AG, A6, IBC, ICA, ICB | chr4 | 55670476 |
| rs2071559 | rs2071559 | 0 | 1 | 1 | I1, I3, I5, I6, I6Q, IM, IMD, IC, ICQ, IBC, CYT, OQ, | chr4 | 55687123 |
| | | | | | IWQ, OE, O24, O28, O54, O5E, OEE | | |
| rs2071559 | rs28695311 | 261 | 0.967 | 1 | None | chr4 | 55686862 |
| rs2071559 | rs2219469 | 22059 | 0.934 | 0.966 | CYT, OQ, OE, O24, O28, O54, O5E, OEE, AAH | chr4 | 55709182 |
| rs2071559 | rs6837695 | 23474 | 0.934 | 0.966 | None | chr4 | 55710597 |
| rs2071559 | rs4864956 | 23475 | 0.934 | 0.966 | None | chr4 | 55710598 |
| rs2071559 | rs7686613 | 24355 | 0.934 | 0.966 | None | chr4 | 55711478 |
| rs2071559 | rs13143757 | 25601 | 0.934 | 0.966 | None | chr4 | 55712724 |
| rs2071559 | rs58309017 | 30639 | 0.934 | 0.966 | None | chr4 | 55717762 |
| rs2071559 | rs2412637 | 31079 | 0.934 | 0.966 | None | chr4 | 55718202 |
| rs2071559 | rs7679993 | 31251 | 0.934 | 0.966 | None | chr4 | 55718374 |
| rs2071559 | rs7680198 | 31371 | 0.934 | 0.966 | None | chr4 | 55718494 |
| rs2071559 | rs7675314 | 31397 | 0.934 | 0.966 | None | chr4 | 55718520 |
| rs2071559 | rs1458829 | 23705 | 0.901 | 0.965 | AG, AAH | chr4 | 55710828 |
| rs2071559 | rs7696256 | 31381 | 0.901 | 0.965 | None | chr4 | 55718504 |
| rs2071559 | rs17712245 | 31697 | 0.901 | 0.965 | None | chr4 | 55718820 |
| rs2071559 | rs1380057 | 2691 | 0.87 | 0.964 | None | chr4 | 55684432 |
| rs2071559 | rs1580217 | 24908 | 0.87 | 0.964 | AAH | chr4 | 55712031 |
| rs2071559 | rs1580216 | 24917 | 0.87 | 0.964 | None | chr4 | 55712040 |
| rs2071559 | rs2125493 | 28573 | 0.87 | 0.965 | None | chr4 | 55715696 |
| rs2071559 | rs1547512 | 30130 | 0.87 | 0.965 | I3, I5, I6, I6Q, IM, IMD, IC, ICQ, CYT, IWQ, O54, O5E | chr4 | 55717253 |
| rs2071559 | rs1547511 | 30302 | 0.87 | 0.965 | None | chr4 | 55717425 |
| rs2071559 | rs62304733 | 31833 | 0.87 | 0.965 | None | chr4 | 55718956 |
| rs2071559 | rs6554237 | 32995 | 0.87 | 0.965 | None | chr4 | 55720118 |
| rs2071559 | rs17081840 | 31813 | 0.869 | 0.932 | None | chr4 | 55718936 |
| rs2071559 | rs7667298 | 635 | 0.84 | 0.963 | AxM, ICA, ICB | chr4 | 55686488 |
| rs2071559 | rs11936364 | 26887 | 0.837 | 0.93 | None | chr4 | 55714010 |
| rs2071559 | rs9994560 | 539 | 0.81 | 0.962 | None | chr4 | 55686584 |
| rs2071559 | rs1350542 | 22027 | 0.806 | 0.928 | None | chr4 | 55709150 |
| rs2071559 | rs1350543 | 22023 | 0.777 | 0.926 | None | chr4 | 55709146 |
| rs2071559 | rs55713360 | 299 | 0.764 | 1 | None | chr4 | 55686824 |
| rs2071559 | rs1380069 | 10416 | 0.743 | 0.891 | None | chr4 | 55697539 |
| rs2071559 | rs11722032 | 32332 | 0.7 | 0.957 | CM | chr4 | 55719455 |
| rs2071559 | rs36104862 | 25268 | 0.568 | 0.906 | None | chr4 | 55712391 |
| rs2071559 | rs12502008 | 1324 | 0.56 | 1 | IMD, OQ, AxM, OE, O24, O28, O54, O5E, OEE | chr4 | 55685799 |
| rs2071559 | rs7693746 | 33631 | 0.542 | 0.902 | None | chr4 | 55720754 |
| rs2071559 | rs1380061 | 33759 | 0.542 | 0.902 | I2, I5, I6, I6Q, IM, IMD, IWQ | chr4 | 55720882 |
| rs2071559 | rs1380062 | 33823 | 0.542 | 0.902 | AAH | chr4 | 55720946 |
| rs2071559 | rs1380063 | 33826 | 0.542 | 0.902 | AxM | chr4 | 55720949 |
| rs2071559 | rs1380064 | 34096 | 0.542 | 0.902 | AAH | chr4 | 55721219 |
| rs2071559 | rs4241992 | 34235 | 0.542 | 0.902 | None | chr4 | 55721358 |
| rs2071559 | rs4864957 | 34360 | 0.542 | 0.902 | None | chr4 | 55721483 |
| rs2071559 | rs4864958 | 34499 | 0.542 | 0.902 | None | chr4 | 55721622 |
| rs2071559 | rs10517342 | 10492 | 0.52 | 0.947 | AX, I3, I5, I6, I6Q, IM, IMD, IC, ICQ, CYT, OQ, AxM, | chr4 | 55697615 |
| | | | | | IWQ, OE, O24, O28, O54, O5E, OEE | | |
| rs2071559 | rs7662807 | 33311 | 0.519 | 0.899 | None | chr4 | 55720434 |
| rs2071559 | rs75208589 | 34402 | 0.505 | 0.856 | None | chr4 | 55721525 |
| rs2071559 | rs74866484 | 34401 | 0.504 | 0.855 | None | chr4 | 55721524 |
| rs2071559 | rs11935575 | 27230 | 0.501 | 0.945 | None | chr4 | 55714353 |
| rs2071559 | rs1458822 | 35450 | 0.497 | 0.895 | None | chr4 | 55722573 |
| rs2071559 | rs9312658 | 13160 | 0.479 | 1 | None | chr4 | 55700283 |
| rs2071559 | rs73236109 | 15059 | 0.479 | 1 | None | chr4 | 55702182 |
| rs2071559 | rs1903068 | 16111 | 0.479 | 1 | None | chr4 | 55703234 |
| rs2071559 | rs4516787 | 17799 | 0.479 | 1 | AX, A6, I1 | chr4 | 55704922 |
| rs2071559 | rs6816309 | 35152 | 0.475 | 0.891 | AH, I3, I5, I6, I6Q, IM, IMD, IC, ICQ, CYT, OQ, IWQ, | chr4 | 55722275 |
| | | | | | OE, O24, O28, O54, O5E, OEE, AAH | | |
| rs2071559 | rs6833067 | 35170 | 0.475 | 0.891 | None | chr4 | 55722293 |
| rs2071559 | rs6811163 | 35211 | 0.475 | 0.891 | None | chr4 | 55722334 |
| rs2071559 | rs1458823 | 35472 | 0.475 | 0.891 | CM | chr4 | 55722595 |
| rs2071559 | rs4356965 | 33250 | 0.464 | 0.894 | A6, OQ, OE, O24, O28, O54, O5E, OEE | chr4 | 55720373 |
| rs2071559 | rs12331507 | 19523 | 0.456 | 0.939 | AN, A5, A6, I2, I5, I6, I6Q, IM, IMD, OQ, AxM, IWQ, | chr4 | 55706646 |
| | | | | | OE, O24, O28, O54, O5E, OEE, AAH | | |
| rs2071559 | rs12646502 | 35677 | 0.453 | 0.886 | None | chr4 | 55722800 |
| rs2071559 | rs1551641 | 1549 | 0.443 | 1 | None | chr4 | 55688672 |
| rs2071559 | rs1551642 | 1844 | 0.443 | 1 | None | chr4 | 55688967 |
| rs2071559 | rs1551643 | 1860 | 0.443 | 1 | IBC | chr4 | 55688983 |
| rs2071559 | rs1551645 | 1948 | 0.443 | 1 | None | chr4 | 55689071 |
| rs2071559 | rs17773813 | 16603 | 0.437 | 0.937 | None | chr4 | 55703726 |
| rs2071559 | rs78025085 | 34403 | 0.437 | 0.769 | None | chr4 | 55721526 |
| rs2071559 | rs6842494 | 14507 | 0.425 | 0.887 | None | chr4 | 55701630 |
| rs2071559 | rs12331597 | 5548 | 0.409 | 1 | None | chr4 | 55692671 |
| rs2071559 | rs17773240 | 7415 | 0.409 | 1 | None | chr4 | 55694538 |
| rs2071559 | rs28411232 | 7643 | 0.409 | 1 | None | chr4 | 55694766 |
| rs2071559 | rs12331471 | 9143 | 0.409 | 1 | None | chr4 | 55696266 |
| rs2071559 | rs9312655 | 10299 | 0.409 | 1 | AX, I3, I5, I6, I6Q, IM, IMD, IC, ICQ, IWQ, O54, O5E | chr4 | 55697422 |
| rs2071559 | rs10012589 | 10323 | 0.409 | 1 | None | chr4 | 55697446 |
| rs2071559 | rs10012701 | 10410 | 0.409 | 1 | None | chr4 | 55697533 |
| rs2071559 | rs9312656 | 10606 | 0.409 | 1 | None | chr4 | 55697729 |
| rs2071559 | rs9312657 | 11136 | 0.409 | 1 | None | chr4 | 55698259 |
| rs2071559 | rs12505096 | 13736 | 0.409 | 1 | I3, I5, I6, I6Q, IM, IMD, IC, ICQ, CYT, OQ, IWQ, OE, | chr4 | 55700859 |
| | | | | | O24, O28, O54, O5E, OEE, AAH | | |
| rs2071559 | rs12498317 | 14727 | 0.409 | 1 | I2, I5, I6, I6Q, IM, IMD, IWQ, O54, O5E | chr4 | 55701850 |
| rs2071559 | rs28838369 | 14883 | 0.409 | 1 | None | chr4 | 55702006 |
| rs2071559 | rs28680424 | 16879 | 0.409 | 1 | None | chr4 | 55704002 |
| rs2071559 | rs73236111 | 26702 | 0.409 | 1 | None | chr4 | 55713825 |
| rs2071559 | rs9997685 | 30537 | 0.409 | 1 | None | chr4 | 55717660 |
| rs2071559 | rs1551644 | 1900 | 0.4 | 0.931 | None | chr4 | 55689023 |
| rs2071559 | rs17711320 | 7687 | 0.392 | 1 | None | chr4 | 55694810 |
| rs2071559 | rs10517343 | 9901 | 0.392 | 1 | AX | chr4 | 55697024 |
| rs2071559 | rs13134246 | 35969 | 0.378 | 0.824 | None | chr4 | 55723092 |
| rs2071559 | rs13134290 | 36037 | 0.378 | 0.824 | None | chr4 | 55723160 |
| rs2071559 | rs13134291 | 36040 | 0.378 | 0.824 | None | chr4 | 55723163 |
| rs2071559 | rs13134452 | 36059 | 0.378 | 0.824 | None | chr4 | 55723182 |
| rs2071559 | rs10020668 | 4537 | 0.376 | 1 | A6 | chr4 | 55691660 |
| rs2071559 | rs10013228 | 4974 | 0.376 | 1 | IM, IMD, CYT, OQ, OE, O24, O28, O54, O5E, OEE | chr4 | 55692097 |
| rs2071559 | rs28584303 | 5256 | 0.376 | 1 | None | chr4 | 55692379 |
| rs2071559 | rs12331538 | 5720 | 0.376 | 1 | AG, AxM | chr4 | 55692843 |
| rs2071559 | rs35729366 | 34921 | 0.368 | 0.743 | None | chr4 | 55722044 |
| rs2071559 | rs28517654 | 1102 | 0.36 | 1 | None | chr4 | 55688225 |
| rs2071559 | rs73236106 | 4346 | 0.345 | 1 | None | chr4 | 55691469 |
| rs2071559 | rs17711225 | 6819 | 0.345 | 1 | None | chr4 | 55693942 |
| rs2071559 | rs9284955 | 10751 | 0.345 | 1 | AX, AN, A6 | chr4 | 55697874 |
| rs2071559 | rs1380068 | 11341 | 0.345 | 1 | None | chr4 | 55698464 |
| rs2071559 | rs1350545 | 12088 | 0.345 | 1 | AN, A5, A6, CM | chr4 | 55699211 |
| rs2071559 | rs9998950 | 4479 | 0.329 | 1 | None | chr4 | 55691602 |
| rs2071559 | rs62304743 | 34741 | 0.322 | 0.617 | None | chr4 | 55721864 |
| rs2071559 | rs2239702 | 227 | 0.315 | 1 | IM, IMD, IBC | chr4 | 55686896 |
| rs2071559 | rs41408948 | 341 | 0.315 | 1 | None | chr4 | 55686782 |
| rs2071559 | rs73236104 | 1412 | 0.315 | 1 | None | chr4 | 55685711 |
| rs2071559 | rs10026340 | 5770 | 0.315 | 1 | None | chr4 | 55692893 |
| rs3025033 | rs3025033 | 0 | 1 | 1 | I3, I5, I6, I6Q, IM, IMD, IC, ICQ, CYT, IWQ | chr6 | 43859053 |
| rs3025033 | rs3025030 | 488 | 0.943 | 1 | IMD, IBC, O54, O5E, ICA, ICB | chr6 | 43858565 |
| rs3025033 | rs3025029 | 519 | 0.943 | 1 | None | chr6 | 43858534 |
| rs3025033 | rs3025039 | 1461 | 0.943 | 1 | None | chr6 | 43860514 |
| rs3025033 | rs3025040 | 1976 | 0.83 | 0.938 | I1, IM, IMD | chr6 | 43861029 |
| rs3025033 | rs6899540 | 7249 | 0.42 | 0.685 | AN, A5, A6, IMD, CM | chr6 | 43866302 |
| rs3025033 | rs78807370 | 10016 | 0.42 | 0.685 | None | chr6 | 43869069 |
| rs3025033 | rs73416585 | 13885 | 0.39 | 0.678 | None | chr6 | 43872938 |
| rs3025033 | rs9472126 | 14225 | 0.363 | 0.671 | None | chr6 | 43873278 |
| rs3025033 | rs12204488 | 13284 | 0.325 | 0.715 | I3, I5, I6, I6Q, IM, IMD, IC, ICQ, CYT, OQ, CM, IWQ, | chr6 | 43872337 |
| | | | | | OE, O24, O28, O54, O5E, OEE, AAH | | |
| rs3025039 | rs3025039 | 0 | 1 | 1 | None | chr6 | 43860514 |
| rs3025039 | rs3025030 | 1949 | 1 | 1 | IMD, IBC, O54, O5E, ICA, ICB | chr6 | 43858565 |
| rs3025039 | rs3025029 | 1980 | 1 | 1 | None | chr6 | 43858534 |
| rs3025039 | rs3025033 | 1461 | 0.943 | 1 | I3, I5, I6, I6Q, IM, IMD, IC, ICQ, CYT, IWQ | chr6 | 43859053 |
| rs3025039 | rs3025040 | 515 | 0.883 | 0.939 | I1, IM, IMD | chr6 | 43861029 |
| rs3025039 | rs6899540 | 5788 | 0.375 | 0.666 | AN, A5, A6, IMD, CM | chr6 | 43866302 |
| rs3025039 | rs78807370 | 8555 | 0.375 | 0.666 | None | chr6 | 43869069 |
| rs3025039 | rs73416585 | 12424 | 0.348 | 0.659 | None | chr6 | 43872938 |
| rs3025039 | rs9472126 | 12764 | 0.323 | 0.652 | None | chr6 | 43873278 |
| rs2305948 | rs2305948 | 0 | 1 | 1 | AG, A6, I3, I5, I6, I6Q, IM, IMD, IC, ICQ, IBC, OQ, | chr4 | 55674315 |
| | | | | | AxM, CM, IWQ, OE, O24, O28, O54, O5E, OEE, AAE | | |
| rs2305948 | rs2305949 | 898 | 0.429 | 1 | I3, I5, I6, I6Q, IM, IMD, IC, ICQ, IBC, IWQ | chr4 | 55675213 |
| rs2305948 | rs34945396 | 3226 | 0.321 | 0.866 | None | chr4 | 55677541 |

The “Coordinate_HG18” designation in the last column of the table provides the position number for each SNP in Build 36 of the human genome, also referred to as NCBI36/hg18 (see, World Wide Web uniform resource locator (URL) address “snp-nexus.org/guide.html”)

### Example 4

### Non-Limiting Examples of Certain Embodiments

Provided hereafter are non-limiting examples of certain embodiments of the technology.

A1. A method for determining a genotype for a subject, comprising: determining a genotype of one or more genetic marker alleles at one or more genetic marker loci associated with (i) a level of ocular VEGF and/or (ii) a VEGF suppression response to an anti-VEGF treatment (e.g., VEGF suppression time), for nucleic acid from a subject.

A1.1. The method of embodiment A1, wherein the subject has been observed to have one or more indicators of age-related macular degeneration (AMD).

A1.2. The method of embodiment A1.1, wherein the AMD is wet AMD.

A1.3. The method of any one of embodiments A1 to A1.2, wherein the subject has been observed to have one or more indicators of choroidal neovascularization (CNV).

A1.4. The method of any one of embodiments A1 to A1.3, wherein the subject has been diagnosed as having AMD.

A1.5. The method of embodiment A1.4, wherein the subject has been diagnosed has having wet AMD.

A1.6. The method of embodiment A1.4 or A1.5, wherein the subject has been diagnosed as having CNV.

A1.7. The method of any one of embodiments A1 to A1.6, wherein the one or more genetic marker alleles are associated with an ocular VEGF suppression response to a treatment that suppresses ocular VEGF.

A1.8. The method of embodiment A1.7, wherein the VEGF suppression response is a VEGF suppression time.

A1.9. The method of any one of embodiments A1 to A1.8, wherein the genotype comprises two or more alleles for each of the one or more genetic marker loci.

A2. The method of any one of embodiments A1 to A1.8, wherein the genotype comprises two or more alleles for each of two or more genetic marker loci.

A3. The method of any one of embodiments A1 to A2, wherein at least one of the one or more genetic marker loci is a single-nucleotide polymorphism (SNP) locus.

A3.1. The method of embodiment A3, wherein the genotype comprises one or more SNP alleles at two or more SNP loci.

A4. The method of embodiment A3.1, wherein the genotype comprises two or more SNP alleles at each of the two or more SNP loci.

A4.1. The method of any one of embodiments A3 to A4, wherein the SNP locus or one or more of the SNP loci are in SEQ ID NOs: 1, 2, 3 and/or 4.

A4.2. The method of any one of embodiments A3 to A4.1, wherein the SNP locus or one or more of the SNP loci are in SEQ ID NO: 1.

A5. The method of any one of embodiments A3 to A4.2, wherein the SNP locus or loci are chosen from rs1870377, rs2071559, rs3025033, rs3025039, a SNP allele in linkage disequilibrium with an allele of one or more of the foregoing SNP loci, or combination thereof.

A6. The method of any one of embodiments A3 to A4.2, wherein the SNP locus or loci are chosen from rs1870377, rs2071559, rs3025033, rs3025039, rs2305948, a SNP allele in linkage disequilibrium with an allele of one or more of the foregoing SNP loci, or combination thereof.

A7. The method of any one of embodiments A3 to A4.2, wherein the SNP locus or loci are chosen from rs1870377, rs2071559, rs3025033, rs3025039, rs2305948, a SNP allele in linkage disequilibrium with an allele of one or more of the foregoing SNP loci, a SNP allele in a polynucleotide that encodes a polypeptide in a VEGF signaling pathway, a SNP allele in a first polynucleotide in operable connection with a second polynucleotide that encodes a polypeptide in a VEGF signaling pathway, or combination thereof.

A8. The method of any one of embodiments A3.1 to A7, wherein the genotype comprises one or more SNP alleles at each of the SNP loci comprising rs1870377 and rs2071559.

A8.1. The method of any one of embodiments A3.1 to A8, wherein the subject has been observed to display one or more indicators of wet AMD, and the genotype comprises one or more SNP alleles at each of the SNP loci comprising rs1870377 and rs2071559.

A9. The method of embodiment A8 or A8.1, wherein the genotype comprises one or more SNP alleles at each of the SNP loci consisting of rs1870377, rs2071559 and one or more SNP alleles in linkage disequilibrium with an allele of rs1870377 or an allele of rs2071559, or an allele of rs1870377 allele and an allele of rs2071559.

A10. The method of embodiment A8 or A8.1, wherein the genotype comprises one or more SNP alleles at each of the SNP loci consisting of rs1870377 and rs2071559.

A11. The method of any one of embodiments A3 to A10, wherein the presence or absence of a thymine allele at rs1870377, or an adenine allele at rs1870377 allele, or a thymine allele and an adenine allele at rs1870377, is determined.

A12. The method of any one of embodiments A3 to A11, wherein the presence or absence of a guanine allele at rs2071559 or an adenine allele at rs2071559, or a guanine allele and an adenine allele at rs2071559, is determined.

A13. The method of any one of embodiments A1 to A12, wherein the nucleic acid is cellular nucleic acid.

A14. The method of embodiment A13, wherein the nucleic acid is from buccal cells.

A15. The method of any one of embodiments A5 to A14, wherein a SNP allele in linkage disequilibrium with another SNP allele is characterized as having an R-squared assessment of linkage disequilibrium of 0.3 or greater.

A15.1. The method of any one of embodiments A5 to A14, wherein a SNP allele in linkage disequilibrium with another SNP allele is characterized as having a D-prime assessment of linkage disequilibrium of 0.6 or greater.

A16. The method of any one of embodiments A5 to A15, wherein a SNP allele in linkage disequilibrium with an allele of rs1870377 is chosen from an allele of rs7677779, rs13136007, rs58415820, rs2305946, rs3816584, rs6838752, rs2219471, rs1870378, rs1870379, rs35624269, rs17085267, rs17085265, rs17085262, rs13127286, rs10016064, rs4864532, rs1458830, rs17709898, rs11940163, rs7671745, rs6846151, rs17085326 and rs7673274.

A17. The method of any one of embodiments A3 to A16, wherein a SNP allele in linkage disequilibrium with an allele of rs2071559 is chosen from an allele of rs28695311, rs2219469, rs6837695, rs4864956, rs7686613, rs13143757, rs58309017, rs2412637, rs7679993, rs7680198, rs7675314, rs1458829, rs7696256, rs17712245, rs1380057, rs1580217, rs1580216, rs2125493, rs1547512, rs1547511, rs62304733, rs6554237, rs17081840, rs7667298, rs11936364, rs9994560, rs1350542, rs1350543, rs55713360, rs1380069, rs11722032, rs36104862, rs12502008, rs7693746, rs1380061, rs1380062, rs1380063, rs1380064, rs4241992, rs4864957, rs4864958, rs10517342, rs7662807, rs75208589, rs74866484, rs11935575, rs1458822, rs9312658, rs73236109, rs1903068, rs4516787, rs6816309, rs6833067, rs6811163, rs1458823, rs4356965, rs12331507, rs12646502, rs1551641, rs1551642, rs1551643, rs1551645, rs17773813, rs78025085, rs6842494, rs12331597, rs17773240, rs28411232, rs12331471, rs9312655, rs10012589, rs10012701, rs9312656, rs9312657, rs12505096, rs12498317, rs28838369, rs28680424, rs73236111, rs9997685, rs1551644, rs17711320, rs10517343, rs13134246, rs13134290, rs13134291, rs13134452, rs10020668, rs10013228, rs28584303, rs12331538, rs35729366, rs28517654, rs73236106, rs17711225, rs9284955, rs1380068, rs1350545, rs9998950, rs62304743, rs2239702, rs41408948, rs73236104 and rs10026340.

A18. The method of any one of embodiments A3 to A17, wherein a SNP allele in linkage disequilibrium with an allele of rs3025033 is chosen from an allele of rs3025030, rs3025029, rs3025039, rs3025040, rs6899540, rs78807370, rs73416585, rs9472126 and rs12204488.

A19. The method of any one of embodiments A3 to A18, wherein a SNP allele in linkage disequilibrium with an allele of rs3025039 is chosen from an allele of rs3025039, rs3025030, rs3025029, rs3025033, rs3025040, rs6899540, rs78807370, rs73416585 and rs9472126.

A20. The method of any one of embodiments A3 to A19, wherein a SNP allele in linkage disequilibrium with an allele of rs2305948 is chosen from rs2305949 and rs34945396.

A21. The method of any one of embodiments A1 to A20, wherein determining a genotype comprises obtaining the genotype from a database using a microprocessor.

A21.1. The method of any one of embodiments A1 to A20, wherein determining a genotype comprises obtaining the genotype from a database using a computer.

A21.2. The method of any one of embodiments A1 to A20, wherein determining a genotype comprises determining one or more nucleotides at the one or more genetic marker alleles in nucleic acid from the subject.

A21.3. The method of embodiment A21.2, wherein determining the genotype comprises analyzing a nucleic acid from the subject, or analyzing a nucleic acid derived from the nucleic acid from the subject.

A21.4. The method of embodiment A21.3, wherein the analyzing comprises a sequencing process, a mass spectrometry process, a polymerase chain reaction (PCR) process, or a combination thereof.

A21.5. The method of embodiment A21.3, wherein the analyzing comprises a sequencing process.

A21.6. The method of embodiment A21.3, wherein the analyzing comprises a mass spectrometry process.

A21.7. The method of embodiment A21.3, wherein the analyzing comprises a PCR process.

A21.8. The method of embodiment A21.7, wherein the PCR process is a digital PCR process.

A21.9. The method of any one of embodiments A21.2 to A21.8, which comprises obtaining the nucleic acid from the subject.

A22. The method of any one of embodiments A1 to A21.9, which comprises predicting for the subject, according to the genotype, a VEGF suppression response to a treatment that suppresses a VEGF, thereby providing a VEGF suppression prediction.

A23. The method of embodiment A22, wherein the prediction comprises a VEGF suppression time prediction.

A24. The method of embodiment A23, wherein a genotype comprising two alleles of rs1870377 is determined, and a VEGF suppression time predicted for a genotype comprising homozygous thymine alleles is longer than a VEGF suppression time predicted for a genotype comprising heterozygous adenine and thymine alleles.

A24.1. The method of embodiment A23, wherein a genotype comprising two alleles of rs1870377 is determined, and a relatively high VEGF suppression time is predicted for a genotype comprising homozygous thymine alleles.

A25. The method of embodiment A23, wherein a genotype comprising two alleles of rs2071559 is determined, and a VEGF suppression time predicted for a genotype comprising heterozygous guanine and adenine alleles is longer than (i) a VEGF suppression time predicted for a genotype comprising homozygous adenine alleles, and (ii) a VEGF suppression time predicted for a genotype comprising homozygous guanine alleles.

A26. The method of embodiment A23, wherein a genotype comprising two alleles of rs1870377 and two alleles of rs2071559 is determined, and
(i) a VEGF suppression time predicted for a genotype comprising heterozygous guanine and adenine alleles for rs2071559 and homozygous thymine alleles for rs1870377, is longer than
(ii) a VEGF suppression time predicted for a genotype comprising homozygous guanine or adenine alleles for rs2071559 and homozygous adenine alleles or heterozygous adenine and thymine alleles for rs1870377.

A26.1. The method of embodiment A23, wherein a genotype comprising two alleles of rs1870377 and two alleles of rs2071559 is determined, and

a relatively long VEGF suppression time is predicted for a genotype comprising heterozygous guanine and adenine alleles for rs2071559 and homozygous thymine alleles for rs1870377.

A26.2. The method of embodiment A23, wherein a genotype comprising two alleles of rs1870377 and two alleles of rs2071559 is determined, and a relatively short VEGF suppression time is predicted for a genotype comprising homozygous guanine or adenine alleles for rs2071559 and homozygous adenine alleles or heterozygous adenine and thymine alleles for rs1870377.

A27. The method of any one of embodiments A22 to A26.2, which comprises selecting a dosing interval for the treatment according to the prediction.

A28. The method of embodiment A27, wherein the dosing interval selected is less than or equal to the suppression time prediction for the subject.

A29. The method of any one of embodiments A22 to A28, which comprises selecting a treatment of the AMD according to the prediction.

A30. The method of embodiment A29, wherein the potency of the treatment is inversely proportional to the suppression time prediction for the subject.

A30.1. The method of embodiment A29 or A30, wherein the average VEGF elimination half-life for the treatment is inversely proportional to the suppression time prediction for the subject.

A31. The method of any one of embodiments A22 to A30, wherein the treatment that suppresses a VEGF inhibits association of a VEGF to a native VEGF receptor (VEGFR).

A32. The method of embodiment A31, wherein the treatment comprises an agent that specifically binds to a VEGF.

A33. The method of embodiment A31, wherein the treatment comprises an agent that specifically cleaves a VEGF.

A34. The method of embodiment A31, wherein the treatment comprises an agent that specifically inhibits production of a VEGF.

A35. The method of embodiment A31, wherein the treatment comprises an agent that specifically binds to a VEGFR.

A36. The method of embodiment A31, wherein the treatment comprises an agent that specifically cleaves a VEGFR.

A37. The method of embodiment A31, wherein the treatment comprises an agent that specifically inhibits production of a VEGFR.

A38. The method of any one of embodiments A22 to A30, wherein the treatment comprises an agent that inhibits intracellular signaling of a VEGFR.

A39. The method of embodiment A38, wherein the treatment comprises an agent that inhibits an intracellular protein tyrosine kinase.

A40. The method of any one of embodiments A22 to A39, wherein the VEGF is ocular VEGF and the VEGFR is ocular VEGFR.

A41. The method of any one of embodiments A22 to A40, wherein the VEGF is chosen from VEGF-A, VEGF-B, VEGF-C, VEGF-D, VEGF-E, placental growth factor (PIGF), a splice variant of any one of the foregoing, subtype of any one of the foregoing, or a combination of at least two of the foregoing.

A42. The method of any one of embodiments A31 to A38, wherein the VEGFR is chosen from VEGFR-1(FLT1), VEGFR-2(FLK/KDR), VEGFR-3(FLT4), neuroplilin 1 (NRP1), neuropilin 2 (NRP2), splice variant of any one of the foregoing, subtype of any one of the foregoing, or combination of any two of the foregoing.

A43. The method of any one of embodiments A22 to A42, wherein the treatment comprises an antibody agent or functional fragment thereof.

A44. The method of any one of embodiments A22 to A43, wherein the treatment comprises an ankyrin repeat protein agent or functional fragment thereof.

A45. The method of any one of embodiments A22 to A44, wherein the treatment comprises an aptamer agent or functional fragment thereof.

A46. The method of any one of embodiments A22 to A45, wherein the treatment comprises a soluble VEGFR agent or functional fragment thereof.

A47. The method of any one of embodiments A22 to A46, wherein the treatment comprises a non-signal transducing VEGFR ligand.

A48. The method of any one of embodiments A22 to A47, wherein the treatment comprises administration of an agent chosen from ranibizumab, bevacizumab, aflibercept and pegaptanib.

A49. The method of any one of embodiments A22 to A48, wherein the treatment comprises administration of a photodynamic therapy (PDT), a photocoagulation therapy, or stereotactic radiosurgery, epimacular brachytherapy, or combination of any two or more of the foregoing.

A50. The method of any one of embodiments A29 to A49, wherein a genotype comprising two alleles of rs1870377 and two alleles of rs2071559 is determined, and a treatment predicted to suppress VEGF for a relatively shorter amount of time is selected for a genotype comprising heterozygous guanine and adenine alleles for rs2071559 and homozygous thymine alleles for rs1870377.

A51. The method of embodiment A50, wherein the treatment predicted to suppress VEGF for a relatively shorter amount of time suppresses VEGF for an average time of about 20 days to about 35 days.

A52. The method of any one of embodiments A29 to A49, wherein a genotype comprising two alleles of rs1870377 and two alleles of rs2071559 is determined, and a treatment predicted to suppress VEGF for a relatively longer amount of time is selected for a genotype comprising homozygous guanine or adenine alleles for rs2071559 and homozygous adenine alleles or heterozygous adenine and thymine alleles for rs1870377.

A53. The method of embodiment A50, wherein the treatment predicted to suppress VEGF for a relatively longer amount of time suppresses VEGF for an average time of about 36 days to about 50 days.

A54. The method of any one of embodiments A22 to A53, wherein a genotype is determined prior to administration of the treatment to the subject.

A55. The method of any one of embodiments A22 to A54, wherein a genotype is determined as part of or prior to a treat and extend treatment.

A56. The method of any one of embodiments A22 to A54, wherein a genotype is determined as part of or prior to a pro rata needed (PRN) treatment.

A57. The method of any one of embodiments A1 to A56, wherein the ocular VEGF is retinal VEGF.

### Example 5

### Examples of Polynucleotides

Provided hereafter are non-limiting examples of certain polynucleotides described herein.

SEQ ID NO: 1
>gi|568815594:c55125595-55078259 Homo sapiens chromosome 4,
GRCh38 Primary Assembly
ACTGAGTCCCGGGACCCCGGGAGAGCGGTCAATGTGTGGTCGCTGCGTTTCCTCTGCCTGCGCCGGGCAT
CACTTGCGCGCCGCAGAAAGTCCGTCTGGCAGCCTGGATATCCTCTCCTACCGGCACCCGCAGACGCCCC
TGCAGCCGCGGTCGGCGCCCGGGCTCCCTAGCCCTGTGCGCTCAACTGTCCTGCGCTGCGGGGTGCCGCG
AGTTCCACCTCCGCGCCTCCTTCTCTAGACAGGCGCTGGGAGAAAGAACCGGCTCCCGAGTTCTGGGCAT
TTCGCCCGGCTCGAGGTGCAGGATGCAGAGCAAGGTGCTGCTGGCCGTCGCCCTGTGGCTCTGCGTGGAG
ACCCGGGCCGCCTCTGTGGGTAAGGAGCCCACTCTGGAGGAGGAAGGCAGACAGGTCGGGTGAGGGCGGA
GAGGACCTGAAAGCCAGATCTAACTCGGAATCGTAGAGCTGGAGAGTTGGACAGGACTTGACATTTTGCG
ATCTTTCATTTACCAGTGGGGAAACTGAGGCTCAGAGACTGGCCCAAGATTACCCAGCGAGTCTGTGGTC
GCCTGTGCTCTAGCCCAGTTCCTTTTCTAGGACTCTGGTTTGCGACAGGGACCTCGGCTGGAGCATGTCC
TGAGATGCCGACACACCCTCAGGCTCTTGGGAGGCTGGGGTGGGAAGGCGCCTGGGGTTGGCAGGCAGGA
GGTGCCTCCGCAGGCGAGAACAGGCGGTGAAAAGTTGTCTGGCTGCGCGCAACATCCTAGTCCGGGCCCG
GGGAAGAAAACCTTGCCGGAATCTCAGGCCGGGTCTCCCGGATCGGACGGTACACTCGGTTCTGCCTCTT
TGCGGGACCCGGCCCGTTGTTGTCTTCATGCTCGAACACACTTGCACACCACTGTGTGAAGTGGGGTCTG
GAGCGGAGAGAAACTTTTTTTCCTTCCTTGGTGCAGGACGCCGCTCTCCTTGCAGAGCGAAGAAGGGGGG
GAATAGGGACTTGTCCTGGGGGCTTTGACAGCTTCCCCAAGGGTCTCCAAGTAACAGCCAACTGTCCTGC
GTAAAGCATTGCACATCTTTCAAAGCGCTGTGGTCCTTGGTGTAAGCGCATAGTCAGAAGTTCAAGCTCC
GAAAACCTTTCCTGTGGGCCTTGGTACCTAGCTTTAGTGCCATTCCTTCCTCTCCCTGCCGCCTAAAATT
TCCGTCTCCTTCAATTAGGAACACACACGTTCTTCATGCAATAGCTGTCTGTCTTTTCTTCCTCACTTTC
CTTTCTCTCTCAACCCCTTAGATAATATTTCTTTCCTGCAGCCAGTTTGCTGATATCCAGATTTCCACCC
TTTGCAGGGTGAGAAAGGGGAAAGGGTCAGAGAAAGAAAAAAAAAAAGTCGAATAATTCAGGGAAAAAAA
TTTCTTACTCCCTAAGACAAGAATCACATGTCTTAGAAGACACTCACACCCACATACAGTACCAGGATCA
TCTGTCCATGGTTACTGAATTTTCTTTATAATGACTTGGTTCAACGGGTCCAGTCCACCATGGACACTCA
TTTGTCCCAGACAAGCCCTCTCTCTCCCCCTTTCTGGGCAGAGAATGAAGGTCTGGAACATGTGGTTGCT
CTGTATTCCACAAAGAAGTGAGTTGCTTTTAAGCCTGGGGTGTTTCCTAGCGTAGTAGTAACGGCAGGCC
GGTCGCCCTGAATATAATGGTGAACTTGCCCTTTTGGAGTGCATTACTTGCTTAATTGGATTGGGCTGTA
ATTGGTGCCATCAAATTCTAGAGACAGAGGCACTGTTGTTTTTCCTTCCCGTCTTTGAGCTGGAAGGGTA
ACAGTGCACAAATTAATTAATATTGGTTATGGGATTTGAACATAGAAGGGCTTTTTATTGAGTAGTAGCA
TGTGTACCTCTTACAGTTATTTCTTTAGAACTTTCTGAAGAGTCCAGCTCAAGCTTGCCAATGAAAACGA
ATGACATTTAATGGAGCAAAAACAAAAAACAAAAAACTATGTTGGTCTACAAATATGAATTTGAAGTTAT
TGAGAGCCTTGTTGAATAGATTTTTGTTGTAAACGTGTCTCTAGAATAGTATGGCATAGTCTCAGCTTCC
TATGAATGAAGGACATACCTTTTCTTTTTTAAAATATTTGTTACACAGGAAAGTGTGTCTAGAATGTGAT
CTGTGGCAATAAATTATGAGAGACCTTCAAGAGTTTCTGATTTTGGTAGCCGAGTGGGCACAGTTTATTG
AGAATCATTTTTACTGCCATTTGTTTTCTCACAAGAATGTGCCCAAATAATGGTTTTTTTCTCATTTGGA
TGGCAGTGTGAATTGTACATCATGTTTTCAGCATCTTTCTCAACCTAGTGTTCCCCAGTCAAGTTTGAAA
TCTGTGTTATCCAAATGAATTGTTTTCATTTTCCTTTTCTTAGACAAAGTGGGACTCCAGGTTTCATTTT
GCTTTTAAACATTTTGGTTTTTTGTTTGCCTGTTTTGGGGGCAGTTATTTCTTTCATATTAAAAAGTACT
GTGCAGGCTGGGTGCAGTGGCTCATTCCTGTAATCCCAGCACTTAGGGAAGCAGAGGCAGGAGGATCGCT
TGAGTCCAGGAGTTCAAGAAGTGCCTGGGCAACATAGCGAGACCCCATTCTCTATTTAAAACATAAATGT
AACCCCCGTTCCACGCACAAAGTACTGTGCAAATTAATTAAACATGACCACCCAGACCAGCAACTGTCCA
AGAGTGGCCCATAGACCATCTGTGGTAGGATAATTTGAAATGCTTGTTAAAATGCAGATTTGTAGACCCA
GGGATATTCTGACAGAGTCTAAAGTCTTAAGAACAAAACTGTTCTAAACATAAGTCAGTACCAATGCCAG
TTAATTTCTGAGATATATTGATATAACTTAGTTTCCAGTTTTTTAAAAACCATATTATTGACTTAAAAAC
CATGATATTGACCAGTTATGTCAGTAACTTATTTTGCACATCTGTGTGGTGTGTGAGAACATGTGCAGTC
ACTTATTCATTTTGCCTGCATTTGTTCATATTGGGATCCTCAGATTCAATGCACTGGATGTTTGCACTGG
GTATTTACTTATACTCTCTCTATTTATTCCGTCTCATACTTCGTCCTATTTGTTCATACTCTCTTATTTG
CCCAGCAAGGTCAATGCCAGTTTAGGCCTAGGGAGTCATTTTTTCTTAGTTGATATGACTTAGAAAGCTT
GGGAGCCTGCCCAACATCAATTACTTTTTTAAAGCTGGTATTTTCTAGGTCTTGATATTTATTAAGACCC
TAGCATAGTGGACAATTTTTCTTTCTCTCATGCTTTTTCAACACCTCATAGCTCTTCACATTTAGTTGAC
AGAGAATTCAGTTATCTTGCTGTAGAGTGACCCATGGTGAGGAATCTATGCCATGGTACTTTTCTGGTTC
TTATCCCTTATAGGTAAAGACAAGTTTCTTATGTCTGAAGCTTGATGTCAGGATGAGTTCAGGGCTTTGA
TGAATAAGTTCAGATCTCCCAATTGTAATTCATTAGCATTGCACTTAAAAAAATTTATATACGTTTTTAA
AAAAGGGTAATGCTAATGAATTACAATAGAGAGAAAAGTACATTAGTTTGCATGTATGTGTGAAACTGGG
AAAATTTTTCACGAAAATATTCATATACTTTTTAAAAAAAGGGTAATGCTAATGAATTACAGTAGACAGA
AAAGTATATTAATTTGCACATATGTGTAAAATTGGGAAAATTCCACACATACATAAAAGTATATTAATAT
GCATGTATGTGTGGAATTGGGGAATGTTTTCTCTTCCTCAGTTTCTCTCCCTTGCTTTTAATGTACAGTC
TTTATGAGCCATTATTTCAGCTGTGGCAGTTTGGTTACCAGGGGAAGCGCACTAGAAAATTGATAAAGGA
AAATGAGACAAGGTCATAGATTCTCTCACTCCCTTCAGGGTACGTAGATGAACTATATAAAAATCCGTCT
AAGTGGGATTCGTTAATCAGCAATTTAGTCAAATGTGTACATCCTATGTTCTATAAGAAATGTCAGTGGG
TCCTTTCCCAAGGGAGTGAGATCATCAGATGAAGGTTCATTTGGTTTCAATGTCCCGTATCCTTTTGTAA
GACCTTGAAGTTGGCAATGCAGGAAAACAGGAACTCCACCCTAGCTCCATGAATTGCAGAACTGTTGTGT
TGGTTTATGACCATCTGCCCATTCTTCCTGTTATGACACAGCTTGTGAACTTTTACTGAGAATGGTGAAA
AGTAAATTCCCAGTTTTATACAATGAATTGCTGAAGAGGCCTTTTAAAGTATAGAGTATGCATTGTTTAT
GGAAGGTGTTTCCTATTAGGTCTAACTCAGTGGCAACTACATTCATTTATTTAATTTGTTTCTAGGTTTG
CCTAGTGTTTCTCTTGATCTGCCCAGGCTCAGCATACAAAAAGACATACTTACAATTAAGGCTAATACAA
CTCTTCAAATTACTTGCAGGTAAGGATTCATTCTAGATCTAGATTTCTTGTGTTAAGTAACTGATTGTTT
ATTGAGTGGAAATAATTTCCAGTAGAGCAGAATTATAATAGAGCTTGTAGTAATTGTTCATAAGTGGTGA
GGTTTCTAAGAACTGATGTAATAATGGAAAATGAGAAGAATTTTCTCTCAAAAATTCTGTACAATTTTGC
TGGTGTTTTTATACTATTCTCTGCCAACATGCATACACACACACACACACACACGCACACAAATACACAC
CCACACCCACATTCCAATAACCAGTACAGCCACCTGGCGTATAGTAGACATACGCTCAATAAATATGAAT
GAATAAATGAAGTTGAGGGCATACATTTAAGGAATAGAGTTGAAAAAATTTGGGACTATATTTATTATGC
TTGGTATGATTCTTGAACACTTATTATCCCTTTCCAAAAACTTTGCTTTATAAGAAATTTATTACTATAA
TTACTTAGGCAGTAATATTTAATAGCAATTTAATATTTAGTGGGTAATATTACTGAGCGCATGATCTACA
TAAATAATGGACTTCGGGCCCTGCCTTGATATTCTGGAATGCATCTTTCCCCACTTGCTAGCAAGAAGTC
ATGCTATTGATTTTTGATAACTGGAGAAGTAGACTTCTTTGTCAAGAAGAAGAGGCCTTTAAATTTTGCC
TTTCAACCCTTACCCCAGGACGAAAGATAGAAGACCCTTGGGTTTAACATAGTGATCACACACGAAAGGC
ATGGAGCCTTCTTAGGACCTGTGTGTTTTTGGTAGAGACTGTGACAAGTGGAGGTGATGTTACCCTCCTG
GAAGAGTGCTGGGGGTCCACAAAGGACCTTGGGTAGGTTATTGCCATTGCTTCATACTTGTTGAATACTA
AGCATTAAACCGAATGACATACATCTATTTTAGACTGCAGTATAAAGAATACCCTAGCCCCTTACCAATA
CCCAGCCCTTGGGAAAAAACACAGTAGCAGGTGCTGTTTCTCTAGCTTTACTTGTTTAAGACACATTTCC
CATTAGATTTTCCTTTTACCGACCCTCGATAACAAGGTTATTTGAAATCCCCAAGGATCCCATGCTCCCT
TTTTAAAACTCTGCATAAACATTTCTTATGTTCTGAAAAAAACCATGGAGTGTGTTAAAAGTAACTTCAT
TGATTTAGCTGCAACTTCCTGGAAATTTTAAGTTCTTTGAATGAAGGGCCAATAATGTTACATTCTTCTT
GATGTTGACTATCTTCTTATCTTCCTTGGGGCCTTGTAGAGAAATGCTGCAGTACAAGCCATCTATGTTT
TAATGCGAGGTCCTTACAAGGTCCTGAGGGACTCTTACTTGCACCTCCTTCCTTCCTAACCTCACTTCTT
ACTCCCCTTTGCTCACTCTTACCTGGCTGCTCTGGTTTCCTGGCTGTTCCCTTAATACTCCAGATATGCA
CCTGCTCCAGGGCCTTTCCATGTGCTGTTTTTGCTCCTGTAATACTGCTCTTCATGATGTTCCTATGGCT
AGCTTTATCAAGACCACCTCCTGCAAAATTCTTTACTCTTTTCTTTGTATCTTCTATATTTTTCTCCATA
GTACTAAACACTATCTTTTATACAATAAACTTTCCTTACTTTTTAATTGCCTGTTTTCTCCAGTTAGACT
GAGGTTCCATAAAGGCATTGATTTTTGTCTGATTTGTTCACTGCTCTTTCTCTAGTCCTTAACAAGTTTG
GCACATAGTAGATGCTTAATAGATATTTGTTGAAAGAAAGAATGCATTAATTAATGGAAAACTCAGGAAT
CTTTATAAGTGACTTCTGAAGCTGAGTTTATAACTTTTCATCATATGTCAATCTGACTTGTTGGTAGAAG
ACTTTGTTTTTTTTTTTTTGAGGCAGGGTTGCCCTCTTGCCCAGGCTGAAGTGCAGTGGTGTGATTTTGG
CTCACTGCAACCTCCACCTCCCGGGTTCAAGCAATTCTCATGCCTCAGCCTCCTGAGTAGCTGGGATTAC
AGGCATGCGCCACCACACCTGGCTCATTTTTGTATTTTTAGTAGAGACAGGGTTTTACCATGTTGCCCAG
CCTGGTCTCGAACTCCTGGCCTCAGGTGATCCATCCGCCTTGGCCTCCCAAAGTGCTGGGATTATAGGCA
TGAGCCACCATGCCTGGCCGGTAGAAGACTGACTGTGTCTGTTGAAGAGTTTATTTAAGTTTCAAAACCA
AATTTTCTCTTTTCTTAGAAATAGCCTCACAGTCTGGCACTTCATATTAATACCTCCCTGAAATTAATTT
TTCAGGGGACAGAGGGACTTGGACTGGCTTTGGCCCAATAATCAGAGTGGCAGTGAGCAAAGGGTGGAGG
TGACTGAGTGCAGCGATGGCCTCTTCTGTAAGACACTCACAATTCCAAAAGTGATCGGAAATGACACTGG
AGCCTACAAGTGCTTCTACCGGGAAACTGACTTGGCCTCGGTCATTTATGTCTATGTTCAAGGTAAGTGG
TGAAATAAAATTCATTTCCCACGTCTCTTTACCAGTTATAAAAGACAATAGGCTCAAAGAAGAATTGAGT
ACAACAAAGGGCTTGCTCTAAAGGCTGTTTGCCAAGAGGAATACACACAATTCTTCTCTCCTGAGGCTTT
CTCTGAGAAATAAGACTCATTGATTCTGGAGCTTGGGCCGTGTTACCTCTTTTTTGCCCAGTTAGTTTGG
GTCTGATCTTTGTTTCCAAGGTAAATCTGTGTTCACTGTTGGCCATTGAGACTTATAAAAAGTCTTCCTA
TGTTTGAGAAGAAAACCTAAAATTCTTGAAATCGAGGAAGATTTGGGGGTGAATTATGGAGAAATTTCTG
TGGAGAGATAAGTTATCTACAGCAGAGTAGGAGATTTTCCCAAGAATGCATAGGAAAGCATTTTTTGCCA
AGGGCTCTGGAGTTTTTTGCACATAGGAACCTTTTTTTCTTACTAGTATTTCATAAAAAACAATTCCCAT
ACTCATGTGCAAATAAAGACATTGCTTCAGACTCTTTTCAGGACAATGTTTCTTTCCTTTGCTTGTTTGG
TCTGAGATCTTGGATGATATGCTGTATCTTTCTAGGATGTGCAGTTTGGGATTGATATTATGAAGGCTGA
CTTAACATCCATATAGTATAAAATAAATGTCACACATATTCTGCATTTATAATGAGTTATGCATTCTTTT
GTGTTTCAAAAATCTTACACTATCTTATCTTTTCTGTGAAAACCTAACTTAACTAATGAGATCCCTATGA
TATAAATTTAAGGAATGTAAGGGCTGCATCATAGTTTGGTTGGATGTACCAAATATTTTTCTTTTCAGTG
AAGATAAACAGACATTTTATGTATTTACGTATATGCCTTTTTACATCCCAGAGTATTTGAGACAGGTGAA
GATGACTTAGACTTTTTTCCCAGAAGCAGCTTTTACAGGGCAAGAATTTCATCAGCTTTGGGAAACACAC
TTGCATATCTCTGCTTACATTTCAGTAGTGTAATATGGTCAGTGCAATGAAAAAGTGGAGACCACATCAA
AATAACCTATGCCACTGGATTCACAATGTTTGAGAAATATCTTTGCCCAGAGTAAGCACTGTCAAAGATA
GAATTCTGTGCCCTCCTCCTTCCCTCCACAAGATTTGAAAGAGACAAGGCTCACATCTTGGAGAATTTCT
GGCTCCTTTTGACCTGGCAGTCTTGAGAGATGCAGCTCGGTCAGAAGATTGCAAGGATTTCCTGCTTTCA
GCCTGTCTAGAAATACTACAAGATGAACATCCCCCATATCTCATTATTTACTTCTTCCTAAGTCAGGAAA
CTTGGAGACATGTGAAAATTCATTTCATGAGTTTCAGTAAATATTTTATTTTGAGAGGCTGGGTGGTGGT
TTGGGTTTCTTTTGTTTATTTCCTTTTTTTGAGATACCGAAATAGAATTGATTTACTAAATAGGTTTAGT
CTTACGTCAAAGGGTTAATTTAGCTTCCAAAGGCTTGCTCTGTAAGCAAGTTATGTAATATTTCATAACA
TGTGGATGAAAGGTAGGCAATATTAAGAAGTGGCAATCCCTAGCACTGTTTATTGGTACACTGCCTGTCT
TTGGGTATACCATTAAATTCTGCTTCCTGTCTAAGCTTAAAGTTCTAGGAGTTGGGCTGTCCAAGATTTT
GGCCATGAAGTTAAACAATGGGAAAGGAAACACTGAAGTATTCTCTATGGATAGGTGTTTAATGTCCCCT
CTGGTCGCCACCTTACTTCCCTAGTCTTCTGACCCCATTCTCTTCAGCAATGGATGGAGCCAGGAAGTGA
GCCCTGGCCTCATAAGATAATGGCTATGGCATGTGGTGGGCTAGATTGGCTGCTTTTCTGTGCTTTCCAG
CTGGGAAGGAAATCAAACTTCTGCTGTTGCAGGGAATTAGCTGCCTTTGTCCCCTGTGGTTTAATTAACT
CTTTCTTCACTTTGACTGACTATTATGAAGCACTCTGAGAATGCTTGATGGGATGTGTTGGGCATAGCAA
TGTGAAATGTTATCTCTCTGAGATTTCAAGCATGACTCCACACCACATCATCTCTATCTCTGAGGAATGG
ACTAGGTTTCCAGCAGCATGTTAACATTGTATGAGTAATGTTTGATTGGCCTTGAAATCTTTTTTTTTTT
TTTTTTTTGAGACGGAGTTTTGCTCTTGTTGCCCAGGCTAAAGTGCAGTGGTGCTATCTCAGCTCACTGC
AACTTCTGCCCCCCGGTTCAAATGATTCTCCTGCCTCAGCCTCTGAAATAGCTGGGACTACAGGTGCGTG
CCATCATGCCTGGCTAATTTTTTGTATTTTTCGTAGAGATGGGGTTTTGCCACGTTGGTCAGGCTGGTCT
CAAACTCCTGACCTCAAGTGATCCACCTGCCTCAGCCTCCCAAAGTGCTGGGATTACAGGCGTGAGCCAA
GAACCCAGTCAGAATCTCTTCAGTTTTCTTCTCAGTCTTTGGAGTGGTGACTTTTCAAATGTTTGTCATT
GAAGATATCAATGACTGCTAAATGTTAAACTAAATGCAAAAACAATTAAACATGGTTTTAGAAAGAATCA
TATCCCTAGTCTTCAGAATCTTAAAATGCTCACATGAATGGTCCTCTTGAATAACCAAATTCAAAAGTGT
TAGCTGTTTCCTGTTAATCTAAAGATCCTTTGGGATCCATTCATTTATTTTCATGGAATTTACATTATTT
ACCTAAAGAGAGAGCACATGAGTATTTTAAATATTAGTAAAACTTGTCGGTAAAGTGTATAGATTTAACT
TTAAATTTTAAAGTAAATATTATCCTTCATTTTGAAAAAATTATAATGATTAATCTTTTAAAATGTGAAA
TCTATAAAAATATATTCTGCTTGTCAATAAACCTTGTGAAAGGAGTCAATCTCAATTGGGAGTTTTTTTT
CAAAATTTTTATACACACAGATATATACACATGCATGTGCATGCACAAACACACACACACACATACACAC
ACACCCTCATGTAGCACAGATATCTATCAGCAGAATAATCTGTGGATGCCTTTGGTTGTGTGAGGTGTCC
CTTCCAGTCATTCACTTGTCTGGTTAGAGTTTAGGAACCTGAAAAATGACCAACTTTTCTAGTAAATACT
ATTAACTCATTAATAAAACTAAATTTTCTTCTAGATTACAGATCTCCATTTATTGCTTCTGTTAGTGACC
AACATGGAGTCGTGTACATTACTGAGAACAAAAACAAAACTGTGGTGATTCCATGTCTCGGGTCCATTTC
AAATCTCAACGTGTCACTTTGTGCAGTAAGTTGCATCTCCTCCAATCGTCTCTTAAGTTTTTATAATTTT
AAGCTAATATTAAGATGGGTAACCTGTTTATAATATTCACAATGAGTTTTAAGGATCCTTTAGGAAGGGT
CAAATGCAATGAATAAAACTAATTAGTATTCTTAAAAATAAGATGAATTCTTCAGTGATCATTGTACATG
GCTCTCATTTTTGGTACTGGATTAAATATTTGATATGTCTTTTTATTACCCAGAGATACCCAGAAAAGAG
ATTTGTTCCTGATGGTAACAGAATTTCCTGGGACAGCAAGAAGGGCTTTACTATTCCCAGCTACATGATC
AGCTATGCTGGCATGGTCTTCTGTGAAGCAAAAATTAATGATGAAAGTTACCAGTCTATTATGTACATAG
TTGTCGTTGTAGGTAAGAGGACATTTCCTTTCCATATCATTAATAACATATCCTTGTATTAAGATCTTGG
AGATAACAACATAGAGTGAAGAAGGATATTGAAAAGTATAGGAACTCAGGATATGGTGTTGGGCAATTCA
TCTGCTCTTCTCTACCAAATAAACCCATGTGCAATTGAGGTTGTCTCTTTTCTTGCCAAGATTAAGGAAG
AAAAAGAAAACTTTTTAAAAAAAGGATGAAAGCGAATGGTATTACTCGAGCACATTTTATGAAGAATTCA
ATGTTCAGAGCATTGCTTGCTATCAATTATTTCAATTATGACTATTTTATGGAAACTTCAGCAATTTGCT
AAAGCTGGCCCTACTGGCCTAGGGCTACTGACCACTGAAAGTTTACTACTTTTCTGTCCACTGGGTTACA
ACATCTTTGAGATCTGTGAAGGTAGTGCTTTGTAAACCTCTGTTGGCCATTTTCCTGGGAGCTACCAAGT
ATTGGTGAGGCCTGCAGGGAAAAACAATGTGGCATGTTTTAAAGTTGCATTACTTTAAAAAATAAATCTG
TGCAAAGTTATAGGCTTATTTGCTCTCTCATGTTCTGTTTTTTCAATTTACTTGCTCTAGGGTATAGGAT
TTATGATGTGGTTCTGAGTCCGTCTCATGGAATTGAACTATCTGTTGGAGAAAAGCTTGTCTTAAATTGT
ACAGCAAGAACTGAACTAAATGTGGGGATTGACTTCAACTGGGAATACCCTTCTTCGAAGGTAACGCTAA
TGATTCAAAGCCAGACCTCCAAATACTTAGATAATAAGCCCCAGTGAAGTTTGCTTGAGAGATAGGGGCC
TCTTTGGCCAGATAAAATGTAAGAGCCTTAAACACACACACATACACACCCACTCACACACACATACACA
CACACACAATTTAAGGGAATTGCAGAACAGATAGCACCCACCAAAAGGTGAAATACCAGGAATTTTGTCC
TATTCTGCAATAGCCAGGCTATGAATATTAGTTTTCTCTAGGTGATTACATCTTTCCACATTATGTCATT
TCTCTGTTCTCCAAAGTTTTTGATCTACATTCCTTTTAAGGGAATTTCTCTTTAAGAGGTGGCATGAGAT
ACACTGCTCCTTAAACAGTGGTCACATTTACTTGTGTTTCTGCAGTTTATATCCATCTCACTTTCACCAC
GTGAGGTTTTAAAAATCCTAATTCAGTTGGTTCCATTTATTTCTCCTGAAACAAAATATATTTGTTGTCT
GCATGAGGTTAAAAGTTCTGGTGTCCCTGTTTTTAGCATTAAATAATGTTTACCAAAGCCCAGATTTAAT
TCTGTGTGTTACTAGAAGTTATTGGGTAATGTTATATGCTGTGCTTTGGAAGTTCAGTCAACTCTTTTTT
TCAGCATCAGCATAAGAAACTTGTAAACCGAGACCTAAAAACCCAGTCTGGGAGTGAGATGAAGAAATTT
TTGAGCACCTTAACTATAGATGGTGTAACCCGGAGTGACCAAGGATTGTACACCTGTGCAGCATCCAGTG
GGCTGATGACCAAGAAGAACAGCACATTTGTCAGGGTCCATGGTAAGCTATGGTCTTGGAAATTATTCTG
TGCCTTGACAAGTGAGATAATTTAAATAAATTTAGGTCACTTAGTGATTCCTATTTTGTTCATTCAGAAG
ATAGTTTCTAGTTTTTCTTGTTAGGGAGGCCACATGACCTAGAGGTCAAGAGCATAGCTTTGTAGTCAGG
AACTTGGGTTCAAACCTCAACTTTAAAGATGAGATGTGCTGATATACAGTAAGAGTTCATTTAGTATTAC
TTATTATAGTTATTGCTGCTATTAGGATTGTTACTATGATAAATAGTATTAGCTAAGGTAGTTTTTAAAT
TTTCATTTTATTGCAAGGCTGAGAGGCCTACTTGAATAAGCATGAGCTTTGCAAACTGGGGAAACATTTA
GCAATATACAGTTGACCTGTGAGCAACTCAGGGATTGGGGGAACTCAGGGGAGTTCCCCTAACTTTCCCT
CCTCTGCAGTCAAAAATCCATGTATAGGCCGGGCGCGGTGGCTCACGCCTGTAATCCCAACACTTTGGGA
GTCTGAGGTGGGTGGATCACCTGAGATCAGGAGTTCGAAACCAGCCTGGTCAACATGGTGGAACCCCATC
TCTACTAAAAATCCAAAAAATTAGCCTGGTGTGGTGGTGGGAGCTTGTAATCCCAGCTACTCAGGAGGCT
GAGGCAGGAGAATTGCTTGAACCCAGGAGGTGGAGGTTGCAGTGAGCCAAGATCGTGCCATTGTACCCCA
GCCTGGGCAACAAGAGTGAAACTCCTTCTCAAAAAAAAAAAAAAAAAAAAAATCAAGGTATAACTTTTGA
CTTCCACAAAACATAACTAATGGCCTACTGTTGACTGGAAGCCCTACTGATAACATAAACAGTCAATTAA
CACATATTTTATATGTTATATGTATTATATACTGTATTCTTCCAATAAAGCTAGAGAAAAGAAAATGTTA
TTAAGAAAATTGTAAGGAAGAGAAAATATATTTACTATTCATTAAGTGTAAGTGGATCATCATAAAGGTC
TTCATCCTTGTCTTCACGTTGAGTAGGCTGAGGAAAAGGGGGAAGAGGAGGGGGTGGTTTTGCTGTCTCA
GGGGTGGCAGAGGTGGAAGAAAATCTGCTTATAAGTGGACTCATGTAGTTCAAGTTTGTGTTATTTAAGG
GTCAACTGTAATTGAACTGGAATTAAATTGAACTGGCCTTGAGAAAATCACCTTAATTTTTTGTTTATTC
TCTTTCATTTACATAAATGTCTGAGTTTACATGGTAATTTGTGTGGCATCCTACTTATAAGCCTTGGAAA
GGATTTTGGAGTTTATATTATGAGAATGCATCAATACAGTGAAATTTTAAAAATACCTTAGATAATGCTA
TTTATTAGAGTTGTAATCATAAAAGTGGCAACAACTATAACAAGTATGATTTAGTGAGCACTTACTTTAT
TAGCTCATCTCATCTTTGAAGCTGAGATTGGAACTCAAGTTCCTGACTACAAAGCTATGCTCTTGACCTC
TAGGTCACGTGGCATCCCTAGCAAGAACTTGAAAATTTCTTCTGAATGAACAAAATAGAAATCACTAAGT
GTCCTAAATTTATTTAAATTATTTCACTTGCCAAGATGCACTTGTCAAAATACACAGAGAGAGATGTGCT
CTGGCTTATGTTTTTATAGAATTACTTTTGTTTTCCAGAATACTTCAGGGAAATAGGGGCAGAAATAAGG
AGGTCAGTTGGGAGGCTAATTGCAGTTATCCAAGTGAGAGTTGAGGGGTGGCTTAGACAAGGGTAGTTGA
GGTGGAGGTAGTGAGAGGTGATCTGCTTCTGGATATATTTTGAAGGTAGAGTCAACAGGGTCCGCTGATC
AATTCATTGGTTGTGGAGTATAAGAGAAAAAGAGTGGAAGATGACTCGAGCGTTAGCATGAGCAACTGAG
TAAATGATGGTGTTATTTACTGAGATGGCAAAGATCGAGAAGGCAGTGAGATTTAGGGAAACAGTGTTAG
ATATGTTTATCTGGAGATGCCTGTTAAACATCCAAGTGGAGATATTTAACATATCAACCCGGAACCCAGA
GGAGTCAGGGCAGAAGATAACACATTTAGGAGGTACGTGAATGATACTTTAAACCTGAGGCTAGAGGAAG
GTGTAAATAAAGAGGAGGTCTGAGGACTGAGTCCTGGGGCCTCATGGTGGAAGAGGTGTGTGGAGGCTGT
CATGGGAGCAGAGGAGAAGGAGCACCCAAGCATCCCTGGGGGACTTAGAGAAAGCTGCACAGAGGAGCAA
GTGTTTGAGTTGAGACTTGAGCAATCACTAGGCTTGTGGGAGTGCACTAGCGGGGAGAGAAAAGCAAATG
CAAACACAGGAGGTGTGGGAGAAACACGGGAGGTGTGGGAGAAGCTGAAAAGTGACCCACTGAAAGATAG
TACAGGAAATCTTGGAACTGCAGCTACTCAGACCCTCAAGGTCTTTGACGTTTCACTTGAAATGAAAAAC
TAAATCAAATGACCATTTACAGTAAGTTGACCTTTTTTTTTTTTTATTTTCTTCCAGAAAAACCTTTTGT
TGCTTTTGGAAGTGGCATGGAATCTCTGGTGGAAGCCACGGTGGGGGAGCGTGTCAGAATCCCTGCGAAG
TACCTTGGTTACCCACCCCCAGAAATAAAATGGTAACTACTGGAAATAAATGCAAAGCATCATTTCGTGT
GAGAGCAAATCCTTTGACTATACTAATTCCTGAGAATTTTTTTTCATAGGTATAAAAATGGAATACCCCT
TGAGTCCAATCACACAATTAAAGCGGGGCATGTACTGACGATTATGGAAGTGAGTGAAAGAGACACAGGA
AATTACACTGTCATCCTTACCAATCCCATTTCAAAGGAGAAGCAGAGCCATGTGGTCTCTCTGGTTGTGT
ATGGTGAGTCCATTCAATTTTCCTCTCTGCCCAAGATTTATTATGATACATTGTCTTCCAAATCAGCCAA
ACCACCGTTCCTCTGCCTCCTGCTGCTTCACTCATATCATGGCTGGGCCTGCGTACAAAAGTCATCTGGC
GTGGTGAAGCTGAAGTGAAACGTAGGACCATGTGCTCTGGCCATGTTTGTTTAAGAGGCCGTGTAAATGA
GCTTTGTGGTGGACAAATGCAAGATTAAAGTAGTGATACCCTCGATAGCTAAATGTTGTGAAATAAGAAT
GCCCACAGGGACAGTTGTCAAGCTAAGTTATACTACCATGTTCCCCTCTCATGGAATTGCCCACCTGGTA
CACAGATGTGTAAGACCCTTCTCCTTAGATTTTGTGCAAAGCTTCTAGTTTGATGTTGTAGTTGATGTAT
CAGAGATGTGCAGGCACGTTCCAACTCTGAAGGCTTTTGAAGTTGACACTGTTGGCTTGGTTGGGAGCTT
TTCTTTTTTCCTTTTTGACAGGAGTTCAGGATCTGATTTTGAGTCTGTAAAGGAAAGATAGTAAGTTTTT
GATGTAAAGATAATTTGAACTTTGTTTTCTGAAACTGAAAGGTACAAATAAGTGTTTGGAATGGAGTGGG
GAGAAGGGTGCCATGGTCAAGTGAGTGTGAGAGGTGCTAAGGTGATGTGTAGATGTGTAACAGGTTTCTT
TATTGCAGGACTTCGCAGAACCTTTTATATGCTAATGTATATTGGTATTCTCCAGGAGGAGAGACATAGA
GTATTCAAGGTTTAACAAACCTATTTGACCAGAGCACCTTTTTTCCCCTGAGCAAATTCATTAATCTCTC
ACTCCAAACAGTTTGAGAAATGCTTCTCTGTTGTAATTCTTTGTTCCCCCTTCTGGTACGGCATATTAAA
ACTTCAGGATATTTTCCCATGACATTAAGGTGCTTCCCTACGTGTCCTGATACTCTTCTGTAGGCCGCTG
AACTTGGCTTTATTATTTTTTTTCAGGGAATATTTTAAAGATAGGCTGGGTGCCGTGGTTTGCATCTGTA
ATCCCAGCACTTTGGGAGGCCGAGGCGGATGGATCACCTGAGGTCAGGAGTTCGAGACCAGCCTGGCCAA
CATGATGAAAACCCGTCTCTACTAAAAATATAAAAATTAGCCAGGCATGGTGGTGGGCACCTGTAATCCC
AGCTACTTGGGAGGCTGAGGCAGGAGAATCACTTGAACCCAGGAGGTGGAGGTTGCAGATAGCCGAGATC
GCACCATTGTACTCCAGCCTGGTGACAAGAGCAAAACTCCGTCTCAAAAAAAAAGTTAACAGGTTCCAAA
AAGGTTGTTTAGAAGCAGCATAGGTGTAGGGGACTGGGGAGAGGAGAAACTGGAAAGTGTATAAGTAGGA
TGGGAGGAGGAAATGAACAGGAAATAAAAACAAAACACGGACAGCAAATAGCCCATTTCATCAGTTCATG
AAGCCACTAAATATTTTATTCACTTTAGCAAATTCTCTGCTATATGAAATAAACATAAAAAAGAAGTCAA
GTCTTCAAAGCATAATCTGAGGCTTTAGGTTGACAGTAATAAGGAAATAGTTTTGACTTTGGAGTCAAAA
AAGAAAGAAAGGAAAAAGGGAGAGAAGAAAGAAGGAAGTGAGAGAAGGGAGAAGGAAGAAAGGGGAAGAG
GGAAAGGGAGTGGAGAGGGAGGGAGGGAGGAAGAGGGAGAGAGAATGAAAAACTCAGATGATGGTGGCAG
GAATGCATTCTCTAAAGATTTACACCTTCCTTTAACATGAGGTGGTTTACGTGTTTGGGTTCAGAAGTCA
GAGTGTCTAGGTTTGTTCCAGGTTTTGCCGTTCGTTAACTGAGTGACCTTGGGCGAGTCATTTTTTTCTG
TTTCATTTTTTTCTCACGTATAAAGCTGTGGACAGTAATAGTGGTTGTGAGGATTAAGTGAATGAATTCA
TGCAAAGCACTTCAAACAATGCTTGGCACATAATAAATGTATTTACTGTGCTATTTCAGCTGTTTTCTGT
AGCCTTTCCCTGATCTCCTAAACTTGAGAGGACAGAGAGAACTATCTCTGTAATACAGATGAGAGGCACA
GGATTTCAACACTTCCATAAAGTCATTCAGCTTGTTAGTTTATTATTATTATTAGCTTATTGTCATTTTT
ATTTTATTTCGTTACTTTATTCCTTTTTTTTTTTTTTGGTAGAGATGGGGTCTCACCATGTGGCCCAGGC
TGGTCTTGATCTCCTGGGCTTAAGCGATCCACCTACCTTGGCGTCCCAAAATACTGAGATTACAGGCATA
AGCCCCCATGCCTGGCTAGTTGTTATTTTTATGAGTATCACTAGAACTCAGGTCTCTTGTTTCCACATCT
AGGTGTTCTTCGAAAAAGAAAGTGGAAGCAAAATCATATGCTTAAAGAAAGTCAGCTTTAGTTGCTAAAA
TCCTCTATTTCCCATTCTTCAAAGCTGACTGACAATTCAAAAGTTGTTTTTCCCATCTTCAGTCCCACCC
CAGATTGGTGAGAAATCTCTAATCTCTCCTGTGGATTCCTACCAGTACGGCACCACTCAAACGCTGACAT
GTACGGTCTATGCCATTCCTCCCCCGCATCACATCCACTGGTATTGGCAGTTGGAGGAAGAGTGCGCCAA
CGAGCCCAGGTGAGTAAGGCCACATGCTCTTTGCTTTCCTGCCATCTTGCATTTCTTACAGCTGAGCTAT
GATATGACTCCATCCTAAATGGAGAAGCCTAAACCAAAAAAAGTTTTCTCTCAAGAGGTAGCCTGAATCT
CCATCCATCTTTCTCTGTGTCTTACATTTTAGGGGATGTCTTTGCTTGGAGTATCCTCCTTTGGGGTTAG
CTAAGCTCAGCCTTGTTAGGTTAGCCGTGAGGTACACTTCTCCAAACACAGGCTATTTGCTCAGTTTGCT
AATTGCCAGTCTTTGGTTTTTCTCCCGATACCAATCGGCTGGTGAATACCACATCCCTCCTTCTTGTGTG
TGTGAAGATCCATCTCTCAGAGGAAATGCTGATAGATGAGAGGCAGTGATAGACCCAGCCCCAGTCCTCA
GGGTCTCAGGCCCAGCTTATCATGCTCTGACACAAGTCCAGACATCCTTAGGGAAAAACACAACAACAGC
AGCCAACCCACCACCACCCTAAGCAGTCCACTTCCTGTTGTTGTTTTTGAAATGGCCACTATGAGCTTCT
TCCTCAGCTGCTGATCATTTCCTTCACAGAGACCATGGTCCCAGAGAAATTACTTTAAGGAGCCCAGTGG
CTTCTAAGTTTCCTTGCCTTCCTTTGAACTAAATTAACTTGAATTGTCTTGTCGATCCAATTTATGAATG
AAGGTTTATTCCCAGAATAGCTGCTTCCCTCCTGTATCCTGAATGAATCTACCTAGAACCTTTTCCTTCA
TTGTCAATGCCTATTTTTAATTGGCGCCAAGTCTTGTACCATGGTAGGCTGCGTTGGAAGTTATTTCTAA
GAACAGAATAACCAAAGTCTGAATCTTTTCCTTACTCTTGACTCTAATTAAAGAAAAATTAAATCATAAT
ATGCGCTGTTATCTCTTTCTTATAGCCAAGCTGTCTCAGTGACAAACCCATACCCTTGTGAAGAATGGAG
AAGTGTGGAGGACTTCCAGGGAGGAAATAAAATTGAAGTTAATAAAAATCAATTTGCTCTAATTGAAGGA
AAAAACAAAGTGAGTTTGAAGTTTTAAAATTTGAAAATCTCTCTCTCTTTAATGGAAGGATGGTACAATA
ATATGTGAGGCATATTGGAGATTAATAATCAAATAGTCTGGATGATTAAATAGAGCGTATTAAGTCACTT
TGAAAATACCATTGACTTTTAGCAGTACCATTAACTTATTAATAGCTTATCAGAGAAAAATAAAAACATC
TATGACATTAAATCTATGCATCTGTGTAGGGTGATTCTGATTTTATAAACATGAGAATGAAAAAATGTGT
ATCATATCATATTAAAACACATCATTAGTTTCATGGCTTCCAAAGCCCTTTTTATATAATGTGTGAGCTC
CACAGCAGCATAATTATACAAATTGAGTAAATATCCCAAACCTAAAAACCCCAAATCCAAAATGCTCCAG
ATTCTGAACCTTTTTGAGTGCCGACATGGTGCTCAAAGGAAACGCTCGTTGGAGCATTTTGGATTTTCAG
ATTAGGGATGCTCAACTGGTAAGTATACAATGCAAATATTCCAAAATCCAAAAAAAAAAATCCAAAATCC
AAACCACTTTTGGTCCCAAGCGTTTTGAGTAAGGGATACTCAACCTGCAATTGCATAAATTTGAGCGTGT
CCAACCGCTGCAGAAGTGGGAATGGCATAGGCAGGTTGGAGTGATTGTGGAGACTGCTGGACTGAGTGCT
TGTGCACAAACAGCCGCGTTGTTTATGGCCTGGGATTTGTTTTTTCCCCGCACAGACTGTAAGTACCCTT
GTTATCCAAGCGGCAAATGTGTCAGCTTTGTACAAATGTGAAGCGGTCAACAAAGTCGGGAGAGGAGAGA
GGGTGATCTCCTTCCACGTGACCAGTAAGTACTCTTCTCTGGAGGTTTGGGTTGGATCACTCACACAGTG
GGTACTAAGCTATGTAATTCCCTGTTGTTTTTGCCATTCATGTGAGTGGCATGGCATTTAGGAAAGAGGA
CTTGGATTGATCATTGATGCTTTCATTCATAAATTACAACTTCTCAGGTATCTCCTGGGCTTATGTGAAG
TCAGTGCGTCTAACTACACTGGAGAGAGAATGGTTTCACAGATGCTTTAAACCACAAGCTCTGTGTGGTA
TTTACATCTCAGTCTTCAGAGTCTGGCACAGTGCCTGGCTTATTGAGCTTCAGTACATATTGGTGGGCTT
GCTGTGGAACAGTTGATGAGGGTGGGCTTTATGGAGGCAATCAGAAGGACATAGGAGCAGTGCCCTCCCA
ATGCTGCCGATTTTGCCTGTGCATCTTAGTTTTATGGATAAGCTTTAGCTGATTGTGCTGAATGGAATAT
TATAGCCAGGGCTAATTCATTGGCATAAATGTAGCTTTCATATCATTGAGTGTTAGTGTTAATGAAGACC
TAATTTTAAAATTCTGTTAGAATTAGAGATTTTGCTTTGGATTTTTAATATATTAAACATTGCGTAGAGC
TCATAGTGGAGATGTGGTAAATATCTGAGGAATTCGTTTACATTTTCAAGTAATGTGTTTGGCCAAATAA
GATATTTTGGGACCTGAATTGTCTAGTTTGTTTGTCAAGTTGTAGTACATCACCTGGAACGGATAGAGCT
TCATTTCTTTTGGTACTTTGTAGTAGTCTGAAAGCAGCAAGATGATAGTGAGCTGTACCAAGTTAAATCA
CCATTCAATAACTATGGCCTCTTCATTTTAGGGGGTCCTGAAATTACTTTGCAACCTGACATGCAGCCCA
CTGAGCAGGAGAGCGTGTCTTTGTGGTGCACTGCAGACAGATCTACGTTTGAGAACCTCACATGGTACAA
GCTTGGCCCACAGCCTCTGCCAATCCATGTGGGAGAGTTGCCCACACCTGTTTGCAAGAACTTGGATACT
CTTTGGAAATTGAATGCCACCATGTTCTCTAATAGCACAAATGACATTTTGATCATGGAGCTTAAGAATG
CATCCTTGCAGGACCAAGGAGACTATGTCTGCCTTGCTCAAGACAGGAAGACCAAGAAAAGACATTGCGT
GGTCAGGCAGCTCACAGTCCTAGGTAGGGAGACAATTCTGGATCATTGTGCAGAGGCAGTTGGAATGCCT
TAAATGTAGTGCAATTCAGGTGCTATGCAAAGATTACTGTCCTCTAGGAGATTATGTTGTAAACTGGTGC
ACACTTCTTCACCGAAAGTCCTTGAGGAAGAAAGAAGCTAATAATAATGAAATGATATATCGAAAGGAGA
AAATAACAAAACCTGATGATGGAGTAATTCACTAGTATATGCAAGGGATATTAGCTTGAACCAGGGAAAC
TTCTGCCTTATCTTGGGCATCCATTTATTTAAATAGACAAATATTTGTGGAATGCCTGCTATGAGCTAGG
AGAGTGTCAGAAATTCACAGTGGTAAACATGAAGGAAAGGAGGAGAACATAGGCAACCACTGGGAAGTCA
CAGCACAGTGAGGTCTCTGTGTCCATGAGAACAGGAATTGTTCTCTGTTTTGCTCCCTGCTATAGCTCTA
GTCATAGAGCATAGCAGCATATACTAACTGCTCAATAAGGCACCTGCTGCATGAAGAGTGGGATGATGGG
CTGCGTTTAAGACCTAGAAGACTCCATGGGAAGGAAGCTACATTCACTGTCTGTACCTCTGGGTCATCCC
ACATGATCCAGCGTAGCCCAAGGTCAATGGGACGATCACTTCAGTGAGCAGATAGCTCTGTAAATTCCTC
CATAGAGGCACTGTCTACCCCTTGTCTAACCTCATGCCTTGTGCAAAAGCTGGGCAGCCATGGCTTTGTC
TGTGGGAAAATCAGGCAAATTTGGGGAGCGTCTCTTTGTGCCACTTCTCTCCATTTTCTCCTCTTGTGGT
GTCCCTTTCCAATTCCTAGGATATATGTGCCCTCTGTTTTTTTTTTACTGTTAGGAAGGAAATTGCCCAA
GTAAATTCATCTATACCACAGTTTTAGAGGGTAACGTCTTCATCAGAGGCCTTGGCGTATTTGAAGAGGC
ACCTTCTGACAGACACTAGCATAAAGTTCGCTAGTTTTAAGACTCAGGTGTCATAATAAGAGATACTTTG
GGGTCAAGTCATCCCCAGCATCCTTCAAGTCACACCACATAGATCACATGGATTTTCTGTTGGCTTGTCT
GGCTTCAAGGTTATGGCAGAATTGAGAAAGAGATGTGAAGTAGGCTCCTGGCCTAGCTGTGCCCAGAAAA
TATGTGCTCGCAGTTAGCTGCTTTGCTTCCCTAAGGACTCCTAACTTGTTTTCCTAAAACCTATTCTTAG
AAATAGGCTAGAATCCAGTACATTTGCTTAGACTTCAATGTAGTACGCTGTTGAGGTAATCTCATTTTGC
TAAGTGTTGACGTGGATTTTTTCAGCATGATTCCTTTTGATGTTCAGTTGGTTGGGACAAGATATTTCCA
CAGCACTTTGATGATCTGAAGAAAGAATAAATCTAAAGTGTTCTTGTACACTTAAACAAATACTCATGGG
CTTCATTTTCTTTAAATCCAAGACTTCCCTTAGGGTATTGTTGTTTTGTTTGTGTTTTAGTGGAAATAGC
ACTGAACTGGTCTTTTAGCCTCACCAGATTCTGTAAACAGTTCAACTGTTTACTTAGTTGCAGGGACATG
GACAAGTGGTTTAATGTCGCTGAACATCATTTATTTCATCTGTGAGATAACGCTAACAGTCCTATTCTGC
TCATTACATAAGATCACTAGTGAGGAACACAAATTGTGTAAACAAGTTTTATAAGAATTGCCAAATAAAT
GTAAGGCATTATTGGTTGAATGATACTAAAATTTGGCACTTCCAAGAGAAATTTGAAGGGATTCTAGGGT
ATTATTGACTAGAATCTTCATGGGAGGGAAGTTTTCACCTGGGGAGGCTGTGTCTAATTAGAGGAAAAAT
CCATAAAGGTGACCCTGAACCTTTCTTTTGTGATGGGATTACCAGCTAGTATCACTAATATGAATGTTAA
AAGCCATTAATCTGTTTGCAGTGTCCTGACTGACTTGTTTCATTTAACTTTACCCAGTGACCAGTGTATT
TTCCCAGAAGTTAATATATCAACAAGTTCCTTTTTACTAAATTTAAACTGTTTAAAAGTTTGCTGATACC
AGAACCATTTCAAAAGTTATAATTCCATGTTCTGTGATTTTCTTTTTGTGTGTCTAGAGCGTGTGGCACC
CACGATCACAGGAAACCTGGAGAATCAGACGACAAGTATTGGGGAAAGCATCGAAGTCTCATGCACGGCA
TCTGGGAATCCCCCTCCACAGATCATGTGGTTTAAAGATAATGAGACCCTTGTAGAAGACTCAGGTAAAT
AGAATTTGGCTATCACTCTTGGGTTGCAGAACTTTCCCAGGGATGTTATCTAAAAAGCCATATTATTTCT
TGATGTAATGTAGAAAAAAAGCAGTATTGGTGTCCATGACCTGGCTCATTTCACAGACTTAGAATTGGAG
TATGGGGCCCTGTTGAATTTTCATGAAAGCCATATAGGAGATTAGTCAGCAGTAGATCCCATGTGACTCT
ACAGAGTTAGATAATAGAACAAGATGAAGGGCAGCATTTATATTTTCTAAATTTCCCTGAAAAACTTCAC
AGACTACATCATCATAAATGAGAATGATCGTTTTCTTCCTCTGTTAGGCATTGTATTGAAGGATGGGAAC
CGGAACCTCACTATCCGCAGAGTGAGGAAGGAGGACGAAGGCCTCTACACCTGCCAGGCATGCAGTGTTC
TTGGCTGTGCAAAAGTGGAGGCATTTTTCATAATAGAAGGTCAGTGGGATAAAAAAAAATGTGGTACATA
TACACCATGGAATGCTATGCAGCCGTAAAAAGGAATCTGATCATGTCCTTTGCAGCTGCATGGATGGAGC
TGGAAGCCATTATCCTCAGCAAACTAACACAGGAACAGAAAACCAAACGCCACACATTCTCACTTATAAG
TGGGAGCTGAACAATGTGAACACATAGACACAGGGAAGGGAACAACACACACTGGGGCCTACTGTGGGTT
GGGGAGAAGGAGAGCATCAGGAAAAATAGCTAATGCATGCTGGGCTTAATACCTAGGAGATGGATTAATA
GGTGCAGCAAATCACCATGGCACATGTTTACCTGTGTAACAAACCTGAGCATTCTGCACATGTATCCCGG
AACTTAAAAGAAAAAAAGAAGGTCAGTGGGAAGTCATAGATACATCCTGTGGTTTTTGAAGATTAGTTTG
TATCTTATAGACACACATTCACTTTGAATAGGGCAACGACAGATGATTTTTAATATTCTTTGTACTTTGT
AAATTTTCTCAGTGAGTATGTATTCTTTTAACCAGCAAACATAATTAATGTTGTTATAATTCTGCTTGCA
TCACATTTCCTATTCCTGCAGTTCTTATTGTGGAAAAATTCTTAATCAGGCAGGATGAATAGCCTCTTCT
CCCTGATTCTGTCTTTGTTTGAATGGCTTGATTAACTTATAGAAATGATGCCTTTATATTTATTTGGAAA
AACATTAGAATTGCTGCCTAATCATGGCAGTCAATGCTATCCAGATAGTCACAAGGATTCCGAGTTTTAA
TTGGACTAGAGATAATTAAGATTCACTTGTGAACAATAGACCATTGCTCTTCTGACATGGAAAATTTTTG
GTTTTTATCTCAATACGTGTGTATGCAGAAGTGATGTGAAATCTGTCATTTTCTTAGCTAGGAAAAGTAA
TTTGTGGCAGAATATTTTATCTTAAGAAGTATATTCCTATGGCTTTTTTTTTTATAGCCCACCAGGGAAA
GAATAAAACTGTGTTGTGGGGTAAAAGAATGGTATGCAAGGGTAAGAAAGAAGTATGGTGATAGAAGGGA
TCGATGGATTTCTATGAACTCATCCTAACTTGTCTCTCAAAGTCTAGATTTTGGTCCCTTTACTCTGCCA
AATCTATGATGCCAAGTATTGCATCGAGATATGTTGACATATTTTCAAATGTATAAGCTTATTAGCATTT
CATAAACTACACTTGCAAATAAAGATTTCAAAGACCATGGCGGTTTTGTCATTTCCAAAGTGATTCATGT
TTTAGGGCAAATCCGCAGAATGACGTCTAGATTGTCTCTGATGCTCTGCATTACCTCTTGTTGGTGGCCT
GCAGCTGGTTACAGATGCCTAACTAGGTAACACTGGCACAGAGATTATAGTTACTTCTTACCTGGAGTGA
ATGCTAAGAAAGGCAGAGCTAGATATTTAATACTCCTGCTGGGTTCCCAAATGTTATGCGAGAATATTAA
TATACAAACACATAGAAAACAGACTCTTTGAACTTTTTATCCTCTATGTTCAACTGGACTTTTAAATCTG
TGTGTATAAATAGAGAATTACTTCCCTAGGACCACCAGAGAAACAAAATTTACTCCAAGCATAATTGTGC
TTGTCTCTCAATGGTTAAGTTAACTTTTATTTTGCAAACCAATTTGTTACTTATTTTGCAAACCAGTTTC
TTACTTGTCTTCTTCTCTCTTGAGGCCGTAGTGGGCCATCCGCACAGCTTGTGGCCCGGTTTGATTCTCC
TTGCACTCTTCTGATGGGAGGCCCCAAGTGATGACTGCTTCCTTATCATCTCTTTGCTAATCACTCTTAG
TGGAAAGCCTGTTTCTGTATTTTGTTTCTTCCACTCAGAGCTGTCCTCTGAAGCCCTGAGCATCTGCAGC
TTTGCTTGCTGACTTCTAGTTTCCTCTTCTCTTTCCTTTCATGAGTGATTTGAAACTCCCATTACCAGGC
CATGCGTGATGTGCTCATCTTGGCTCTTCCTCTTCTCCTCACTCAGACTCCTGCCACAAGGGATGGGGTA
GTGTATGTAATGGTTAGTTCATGTTGGACAGGCCTCTTTATCTCTTGACTGAACCACTGACTAGCTGTGT
GCCCTCAGTCAAGTAGCTTAAGCTCTCTGGTCTTCTGTTTCTTCATCTGAAAACTGAGAGTTGTTGAGGA
GATTAAGTGGAATGGCATATTTAAAGTGATGAGTGCATAGTAGATACATGGTCATTAGTAACTCTCAGGT
CAAAAAATTTTGTTTATTTCCCTACTTGGTTTCTTATGTGATCCTTTTGCAAACTCTGCACAGATCAAAA
TATTGACTATCAGTTTAAAAGAAGACTTTTGTTTTCCTCAAATAGAAATATTTTTTTTTCTCTGTAGAGA
ATGATCTGTTTTCTTTCCATCAAAGACTGCTCTTCCTCTAAACACTTTCTATGTTTGGCTTTTAAGACAT
TACTACTTCTATGCTTAATTACTTAAGAATTTTATTGTTGTAAGTTTACATGAGCAATGTTTTGCAAGCT
TTAAATTTTCCATTAACAATTCTGTAGGCCAGGTGTGGTGGCTTATGCCTGTAATCCCTGCACTTTGGGA
GGCCAAGGCAGGGGGGATGGCTAGAGGCCAGGAGTTCGAGACTAGCCTGGGCAATGTAGTGAGACCCTGT
CTCTACAGAAAATAAAAGAAAAATTAGCTGGGCTTGGTGGTATGCACCTGTAGTCCCAGCTACTCGGGAG
GCTGAGGGGGGAGAATCGCTTGAGCCTAGGAATTGGAGGCTGCAATAAGCTATGATTGTGTCATGGTACT
CCAGCCTGGAACATAGAAAGAAACCCTGTCTCTAAAAATAAATAAATAAATAAATAAATAAATAAATAAA
TAAATAAATAAATTAAATTCAAAAAAAGAATTCTGTAGACTCCATTCAAGTTACGGGTGTGTAACTGTTG
TCCTCTAGGATTTTTCCAAGTTGGTAAGCTTGGGATTTTGCTTTAGTGCTAAAATTTGTCATCTTACAAA
CAAAAAGTATAAGTTTCCAACTGTTGATACTCATTCAATTGTGTCTTTCCAGGTGCCCAGGAAAAGACGA
ACTTGGAAATCATTATTCTAGTAGGCACGGCGGTGATTGCCATGTTCTTCTGGCTACTTCTTGTCATCAT
CCTACGGACCGTTAAGCGGGTAAAAAAATAATTTCCCTTCTGCCCATGCACATTGGTTTTCATGATTAAT
GAAAACTGACTGGGGTTCTTTGAGTTGTTTCTTCCCATTGTTATTGGCTCAATGGGCACATTTTTATTTC
AATACAATAACGTTCCTGCCCACTTTCTTTTGGCTGGATCTCAGGGATTTAATTGATAGAAGCCACTAGA
GAGGAAAAGGGCTTGGACTGTCTAGTGTAATTAAGCTTTAAAACCTTAATTCTGAGCTCCTTTGGGGGAC
AAGGGAAACTAGAAGCAGGGTTATAATAGGACCACTCTCAAACTCCATGAGTTTTATTGGAAAATGAGAC
AGGAATGAGGCTCCAATAAACAGCAATAACAAGCACACAAAACAACAGCCAAACAACAGTGTGTTTATGA
CTGGAAGGATTGATGCTTTCCAGGCCAATGGAGGGGAACTGAAGACAGGCTACTTGTCCATCGTCATGGA
TCCAGATGAACTCCCATTGGATGAACATTGTGAACGACTGCCTTATGATGCCAGCAAATGGGAATTCCCC
AGAGACCGGCTGAAGCTAGGTGCATTTTCAATTGCTATTAATTTGATATTGTGTTTACCAGGCCATCTCT
TCCTCCATTAGAATGATGACAAATGTGGTGTATTCAGATGTTGGATTCTGGTTTAGAAATATTAATTCCA
TTTCTTGAATTTGTATAATCATTCATATAGCCACTTAGAGGTAGGGTCCCTATGTAATCATCCAAAGCAG
GACATTTGGAGAGTGAAGGGGGAGTTATTAAATAATTAAGCCAGGACAAAGGAGTAAACTGGACTATCCA
TGTTAAATTGGGATGTATGGTCACCCTATCTAGTTGATGTCTCTGCGTATCACTTTGGTTGTATAGTAAT
CCAAGTCTGTTTTCTTGTTGCTGTTGTTGTTGACTCTAGGTAAGCCTCTTGGCCGTGGTGCCTTTGGCCA
AGTGATTGAAGCAGATGCCTTTGGAATTGACAAGACAGCAACTTGCAGGACAGTAGCAGTCAAAATGTTG
AAAGGTAAAAGCAAAATTATGTGGTGATCTATCTTTCTGTTTTATCTAGTCTTTAAATATGTTGCAAGGC
TTGTATCAGTAGCTTTGTGCTTATGTGGGCCTACTAGCCACACATGCAGTCAGCCTAAATAATGCCCTTG
TGCAAATTGGAAAAAGGATCCTCCTTTGTAGCTTTATGCCAGGATGCATGGTCTGGCAAGCAAAGTTGGG
AATGGCTTTCACCTTCTTGCCTGGTTACCCTCGTGCAGGGCTCAGCCAACACAGTTGTACTTAGTGGTTC
TGGGTACAGGGAAAAAGGACTGTGGTTATATTAAAATTGTTTCTTAATATATTGTGGAATCAGATAATTA
TAGACCATCTAGAGACATGGAAAGGAAGATAGTGAAATACAAAAATAGCATGTTCTCCAGAATTGGAATA
TGTAAAAGATGTTCATATGTAAAAGATAATTTGCAAAACAAGAATGGTTGTGTTAGAAAAAAATATAATG
GGTTATATTTTTTAAATTAAAAGCTTTATAAATAATTGTTAATTCTAATAGTAACGGAATTCTGGTCTGG
CCATTTTCATTTTAGGAGGTTAGACAGTAAAGCTTCTTTCTTCAATTGTGATGTTCTTTCATTGATGAAG
GCAGTGCCAATGACCCTTTGCCAATAGGTTTTGTGCATTTCAAAGCTATCTTTCTCCATCTGCCTTTTTT
CTCTTGTGGCCAAGGGAGTGTGTAATTTTGAGGTGGCTCATCAGAGCCTTAGATGTGGACCATGCCTGTG
AATTAGTGGGAAGTGTAGCAGTCCATACAGGATCAAACACATAGTCTTAGTGCCATCAGCCTCATGTGCC
AACTGGTCTTTCCAGCTGGCCTTAATTCGCCTGCACAGATCGGCACAGATTGGCTGGAACATTCGGTATA
GCCCCTAACACGTGAAGATATTTAATACATGGTGTTGCTTCCTTATGAGGAAGTGCTGAAATGATCAGAC
CCTCAGAATCATAGTGAACCTGAAATGCAAAAATCCAGTTTTGCAGAAGAAGAGAATCTGGGCATGATTC
CACTGCAGATGTATTCTCCGCTTTGCAAAAGGTTTCACAATGGGTTCCTTTAAATATCAAACTTTCTGGC
TCACTTAAAATATGAATTTTATTTCAAATTAGAAAATAGAATTTACACTTCACTTTTGAGGAAATGCATG
TGGTCTGTAAACTAGGTCACAGCTGTGTTACCCCGGAGGGTAAGTTGTATAGTGGCATGCAGGGAGGGAG
GGACCCCAATTATTGAAGGAAATGTCCATACCTATGATTTCCCTCTTTGTACTGTATTTGTAGAAGGAGC
AACACACAGTGAGCATCGAGCTCTCATGTCTGAACTCAAGATCCTCATTCATATTGGTCACCATCTCAAT
GTGGTCAACCTTCTAGGTGCCTGTACCAAGCCAGGAGGTGAGTAACTGTGGGTGGTTTTGGTCACCCAAT
TTTAACATGCCTCTCTGATAGTGTTTGAGGGAAAGCAGTCAACTCCTCTGGCCTTGATTTTCTTAGCTTA
GAATACTTTGCGGATTCCTAGGAATAAATATATTTCATGGAGGTTTAATTGGCACTAGAATTAAATTATT
GTAAAACTTTCTCTGAATTAAGAAATGTCATGCTACTATGATACAGTTTGTTACTTGTGTAACAGATGTC
CAGAGAAGAGTAAACTTCCCTAAAACTTGAAAGCTTAAGGGTAGTTACCCCCAAAATGGAATCATATCAG
GAGATTGCACTGAAAAGCAAGTAGATGGGTGGGTTTTCTTCTGAAATTTTGGTTAATCTTGTGAAAATGT
GTTCTGGAAAAAAGAAAAGCTACAATATAAGGGGATTGGGACCAGCTGATTTCTACACTCCTGTCCCAAT
GAAAGGTTGTAGCCTTCTTCTAAGGTGTTTTTGGGTTCATCACTATATTAAACGCTTAGTGAGGAATATG
AGTGAAAACCCATTTTCCTTCCTGGACATGCTGCCTGCAGGGCCACTCATGGTGATTGTGGAATTCTGCA
AATTTGGAAACCTGTCCACTTACCTGAGGAGCAAGAGAAATGAATTTGTCCCCTACAAGGTATGTCATCT
CCTAATCCTGCTCTGGCCATGTTATAAAATGAAGGGAAACTCAAAATGGTACAGGTTAGTTTTTTAGTTG
AAATTTTGTGAAGAACTTGTGAGGAATCTTCTCATATTACCTCTTGGCTGTTGTAACTTCCTCTTTTACC
TTCTGGGGGCCATATGTTTCTGTTTTATGTATGTGATTTTAATCTACTGACCCATTACAGAGTGTGGACA
TGGGGGAGAAGGCAGGTATGAGCGAGGAAAGGGGAGGGCAGAGGGTAGGACATCTCTGGGTTATTCTGTC
TCTCCCCTAGCCATATTTGGCCCCGTGGAGTGTAAATCCCTCTGTGAAGAGCATCCTAATGCTGAAAGTG
TGTCTGAATGCAACTCAAAATGTGGCATTTGTCACTTTAAGCTAAAGAAGGAGCTAGGCTTTGTGGAAGA
AACCCTATTATGCACAAAACTTGCCCCAAGTTTCAGCTCAGAGATTGCATAATCCTGAAATTGATGTCCT
CCTTGTCTGCTTTTTAGTAGTTTCAATTATCTCCATGGTTTACTACATTTTAAAGGTTGTAAACTTTTAA
AGACTCATTTTGTATTCAAGGAGTTTGTTTGTTCCTTTGCTTTTTTATAGACCAAAGGGGCACGATTCCG
TCAAGGGAAAGACTACGTTGGAGCAATCCCTGTGGATCTGAAACGGCGCTTGGACAGCATCACCAGTAGC
CAGAGCTCAGCCAGCTCTGGATTTGTGGAGGAGAAGTCCCTCAGTGATGTAGAAGAAGAGGAAGGTACTG
GCTAGTGCTTCCTGCATGCTATGGCATGCTCTTGTCAGAGCAGACAGGGTGATAGGGTGTTACAAGGAAT
TTGATCATGGGAAAAGTCCAATACTACCTCATAATTTGAAAGAGACCTGAATTTCTATAATAGACTGCCT
CCATTCTGTCTCCCCAAAAGTGAAGTGTGGAAGCCCTAGACTGGGAAGTGAAGCAGGGCTAGCCTGAGAA
ATCTGGGTAGTCCAAGTGGGCTAAGCAGTCGGCTACAACCACAGCAGTGTTCTTAAAATACTGGTTCAGC
ATTTATTAGTGAGAGAGGCCACAAGTTTTCTGGTAGTTGACTAGCCTCTCCATTGCCTTGGAGAGCCCCA
GAGTGGTTTGCCCCACGTTGCATGCTTTACCTGTGCAAAAGTCTTTTCATTATACCTAACCTTCTCAAAG
GCAGTTTAGGAGCCATCTGTTGTTTCTACCCTACCCCAAGCGGCTTATCAAGTCTTCCTTCCAACCATAC
TTCCTCAGGCGAGTCTTGATAAATATCCTGGCCTTTATTAAGTTATGTTTCCAGTGATATTTTATTTATT
TGTTTTTATGTTTATTTTTATTTTTTTGAGGTGGAGTCTCATGCTGTTGCCCAGGCTGGAGTGCAATGGT
GCGATCTCGGCTCACTGAAACCTTCGCCTTTTGGGTTCAAGTGATTCTTGTGCCTCAGCCTTCCGAGTAG
CTGGGATTACAGGTGCCTTCCACCATGCCCAGCTAATTTTTTTTTTTTTTGTATTTTTAGTAAAGATGGG
GTTTCACCATGTTGGCCAGGCTGGTCTCGAACTCCTGATCTCAGGTGATCCGCCTGCCTCAGCCTCCCAA
AGTGCTGGGATTATAGGCGTAAGCCTCCGTGCCTGGCCTGAGTGATATTTTAGTGCTCTTTTTGGGTGGA
GCTGTGGTCCCAGCCTAACTTCCAGGACTTCAGCCGGCTCCAGGACACACTGTATTTCTGCCTCCTTCAG
AAGGAGCAGAGATAGCGTTGTGGATGTAGAGATGGGTGACAGGCTGGCTCCCCTTGAGGCATAAGTCTAG
AAGAATAGTGGAAGAAACCCACTCTGTTTCCCTTGACATGAGGCTACAGAGAGAATTTGCATTTAACTCC
TTTTCCTTAGAAGCTGAGAAGGTAGTGTGAGGCTGGGACTTGGTCTAGAAGCACATGGGGAGGTGGTCTA
GGCTTCATTTAGCTGGGCCCACACTGAGTGGTGCTGCCTCTACCCTGCTCTTTGTCTTTCAAAAAACAGT
GGCCAGTGAGCCAGAAACCTAAGAGATTGAGTTGTTGAGAAAAAGGCTCACAGCCTTTTAAATACTTACG
AATTTATTACTACAACTAAGTTTTTGTTTACTCTGGTATTTGTCTCCAGGAAAGAAGCCATAAGTCTTAT
CTGACCAAAGAGATGATTTTGAAACACCCATTTAATATCTTAGTGTTTATTTGTACCAGTTGCACTGAAG
TAAATACCACCAATTTACGTAAATTTATCTTTCCATGTTTCTGTTATCTCTCAGGAAAAAACACCCTCCC
AGGCCAGATTTAATGTATTTACAGCACTTTTTAAGTTTGAAAATGAATTAAATATATTTCTAGTATTTTT
AGTTATCTATTGCAGATTATAGTTTGACTTTTGGCCTTTGTCCCAGGACAAAACCTGGAGAGAAGAGATT
CAATGACCCTGAATATTGTTGTTTTATTTTTAGAGTTCTTGATATGAAACTATTGTTTATCCCTCTGGGT
ACATGACAAAAAACAGTGTAAGTGGCAAATTTGGAAATGTCCTCTTTATTTCCCAGATTATCTAGGTCAG
TGTTACCTTATTCTACCTCCTGGATTTACTGGTTCAATTTGGCTAAAATGGAAAAACCAGTATTGTTCCT
AAGGGGGTATGATGAAGGCTAATGATACTGGGATTCAGGAGATTTACAGAAGATAGAAGCATTGACTCTC
TGCTTCTATTTCCTAAAAACTTAACTCCCAAGTCTTAAAAAGATTATTACTCTAGCAAACTTAGAAACAT
CACACTAACTCATGGAAATACTGATCTCCATCCTCCTGCCTCTTTGGACAGCTCCTGAAGATCTGTATAA
GGACTTCCTGACCTTGGAGCATCTCATCTGTTACAGCTTCCAAGTGGCTAAGGGCATGGAGTTCTTGGCA
TCGCGAAAGGTAAGAAAGGTTGAGGGGAAATCAGCTATCTTTTCAGATCACAGGTTTGGAAATAAGATGT
CCAGTGTCAGCCATTGGTGCTTGTTTGGGATTGTAATTCATTCACCACTTCTACGTCTTTTAGAAGAGCT
CTACTGGGGAGGCTCTGTTTCTGCTGAGTAAGAGTGGTTAAGGAGTTCATGAAATTAAGCTGTATAATAA
AGGCTTGTCAAGCATCTACTAAGTGTGAGGCAGTCTTCTGAGCACTGAGGATACTGTGGTGAACAATCAG
GCAAAGCTCTTCACCTTCATGGAGTTTACAGTTCTAGTGGGTAGAGCAAACAATAAGCAATATAAACAAG
TAAAACGTGTTGTAGGTTAGATGAGAGTAAATGCTATGGGGAAATAAAGCAAGAAAGGGTTATAGAATAC
ACAGGAGCAATGCACTTGTGTATGTTTATGCTTCTCTGTGTGTGTACATCTACTTTAAACAAGGTAGACG
AGGAAGGCTTTACTAAGAACTTGACATTTGAGCAATGACCTGGAAAGGGGAGGGGCTGAGCCTTACAGAT
ATCTTGGCATGAGAATCATTTTTAATTTATTTTACATTCATCAACATCCATCAAAAAGTATTTGTTAGGA
GTATAATTAGAAACGAGGAAGGACAGGCTTCAGATGAGAGCGATTAAAAGAGCTAAAATTAGAAAAGTAG
GCCAAACAAAGGCTGAGATGGGGACGTGACAAGTTACAACTATTCCAAAGGTTGTAAACACCAAGCGGGG
AGCAAGGCTGGTGGCAGTGATTCCCCTGGAAAGGATAAAAGGTGTAATTTTATATTAGGTAACAATACTT
CAAATTAAGGATCAGGAAGAACTATCAGTTGACAGAATGTATTCATGCAGCTTAATGAAGAAAGAAAGAC
TTAAGTCATATTTTTTTTTGTTTTTCCTAAATTAGAATGAAATCTTCAACCCATGTTTTCCCCTTCTCAT
AGCATTAAAGGCCTCAGGCTCTTTGATGTTTCTGCTAGGTAGCTCTTATGTTCTCTCTCCCAAGGGGAAG
GAGGAGAACTGGGACCTTATAGGGTTTTCCCAAAGAGAAAGGCCCTTTACACTTCTTGGAGATTATGACT
TATTATTACCATTTTTTTATGGCCGGAATTCGCCACTTAGTCAGGGTTCCTTTTGGGGACTAGGAAGAGA
ATGGAAATGAATGTGGGAATGCTTTAACTTTCCTTACATCTACCAGACTATTTCTTGAATCCACTTGGTT
GTCGGGTTAAAAAAGGAAACTTTTTGTTTGGGGGGAAAAGTCAAAAACACTGTCTGTTTTTTGGAATTGC
CAGTGTTGCTCAATTGTGCTAGATAATGTGCTTCTGAATATGCCTTGTTCAGAGGAGAGTGCCATACAGA
TTTGAGGTGTGGGAAGGTCAGCAATGCCTGGCTTACATGATCACTTCTCCAATGATTTAAGAATTCTCCT
TTTGGCCAGGTGTGTTGGCTCATGCCTGTAATTCCAGCACTTTGGGAGGCCAAGGTGTGTGGATCACCTG
AGGTCAGGAGTTTGAGACCAGCCTGGCCACCATGGTGAAACCCCGTCTCTACTAAAAATATAATAATTAG
CTGGGCGTGGTGGCACACCTGTGGTCCCAACTACTTGGGAGGCAGAGGCAGGAGAATCACTTGAACCTGG
GAGGTGAAGGTTGCAGTGAACTGAGATTGCACCACTGCACTCCAGCCTGGGCGAGAGTGAGATTCCTTCT
CAAAAAAAAAAAAAAAAAAAAAAAAAGTTTTCTTCTAAGCCATTGATTCATTTCTTGTGCTCCCCAAGAC
TCATTTTCTTACAAAATATCATGTGGAGCTAAAGCTGCCGAGTAGTAGGAAGTTAGCTGAAGTTTGGAGG
ATACAGAGAAAGGAGAAACTGAGAAGCTAAAAGGAAGAGAAAGAAGTCAAGATGAATCTCATTGTACTAT
TAATGCACTAGAAAATCAACCTGACTTGTGATAGGCTGAAATTGCCTTAATAGACCTTTATAATAACCCA
GCACTTTGAAATCAGGGGAAGCCACATTGGGAATTGTTTATCAGAGCCAGTCTGGCTTCAGCTTCATACG
GAAGGGGGAAACCAACAAAGAGCACTAAACCAATGAGAGCCCCTTGTTTCTGATTTCCGTGCATTCATTC
AAAAAACAAATCCCGTTCTCGGACCTCCTTAGAATAACACGTTTTAAACCAAATATGGGGCCAGGTAAAA
GGAATGTGTGGATGTGACCAGAAACACACTCTTTTGTGTCCTAGAGGAGCCTATTTATGATTCCATCATC
ATATTATAACTTAATTATTTAACTCCAAAGGCTGGGGCTGTTTATGGAATAAGCAGATGTGTGTCTCAGC
AAAGCTCACAGACTTTTTTCCTGAAGTGTTGATAAAAGATACTAACCCAGTCCTTGTTAATCAGTTGGCT
TTCTGATGTGGGATTTTTTTTTGATGCATGAGGTCACAACAGATGTGAAAGAGATCAGCTGTGCCGAGAC
CTAATGCACACATGATTCTCTTTGCAGTGTATCCACAGGGACCTGGCGGCACGAAATATCCTCTTATCGG
AGAAGAACGTGGTTAAAATCTGTGACTTTGGCTTGGCCCGGGATATTTATAAAGATCCAGATTATGTCAG
AAAAGGAGATGTAAGTTTCAAATATGAACCCAGTGCTTGGTTAAGTAACAGAATTAAAACTCCTCGTAGA
GAGCTTCAGGACCTGTGTTCAGGAACAGAGGAAGTTTTTTTCTTCAGATATTTGCTAATTTGGGTTCTGA
ATCCTTGTCTTCTACCCCTGTAGGCTCGCCTCCCTTTGAAATGGATGGCCCCAGAAACAATTTTTGACAG
AGTGTACACAATCCAGAGTGACGTCTGGTCTTTTGGTGTTTTGCTGTGGGAAATATTTTCCTTAGGTAAG
TCATTTCTTTTTGTCCTTCCATCCAGACTCCAAAGAGGAAGACAAAAGTTGTCTTTTCCTCTCCTGTACT
TCATGTCTATCAGGCAAAACTTCTCGGAAGCTTTGAAAAAAAAAATAGATACATAGGTGATGAGGATGTG
CAAGATTCAGGCTCAGGGTTTTCTATAAGAGAAAATCAAATCAAAGAATGTCTCCTCCCTGTTTTATTCT
AGGTGCTTCTCCATATCCTGGGGTAAAGATTGATGAAGAATTTTGTAGGCGATTGAAAGAAGGAACTAGA
ATGAGGGCCCCTGATTATACTACACCAGAAATGTAAGACTTTAAGAAGTATTCCTGTGTTCTCTTTCTTT
GCTCGCAAATTCTCCTTGCCTGGAAGACTTTCCATTATATAGACCTTCTTCATTGCCCAGTTAGTGTCCT
GCTTTTACTTTGGGGCCTTTCTTGATAATTTCAAGCATGGAGTCATCACTTCTTGAAAAGATAGTACTTT
ATTATTCAAAGCAACCAGTTAGTTTTTATTAGATGTTGCTTTAAATGTTTTCTATACACATTGAGCCTCT
GGAGTATGGGACTCTGTGTCTTACACAGTTTTGTATCCTTATTTAGCATCTCACCTCGTCAGCTCTTTAC
AAATGTGTACTCATTTAAGTGCTTATTTTCAGCATTCAGGAAGAAAGAGGCATTTAATGAAATCAGTGTT
TTGCTTCTCTAGGTACCAGACCATGCTGGACTGCTGGCACGGGGAGCCCAGTCAGAGACCCACGTTTTCA
GAGTTGGTGGAACATTTGGGAAATCTCTTGCAAGCTAATGCTCAGCAGGTTTGTCACCTCCATCCAAGAA
GCACCTACAAAGAGTACTTAGATGTCAAGGACTTTCCTACTGCCTGAACTGTCTCATGGCTACCATGCCA
TCCTCTCAGCCATTGAATAATCTACTGTATTCTTCTACATCTGAGTAATAATGCTTTTCTAAAAGCTGTA
ATTACCCTTTTAGACAGATAGGATTCTAATTTATAACCCGGGAGCAGACCACTCTGATTTCTACCTACTT
ATCTTTTTGTTATATTTTCAAATCCTCTTCTAAAGTTAAAACAAAGAAAAAATCTGGTTGATCCACAGAA
GATCAACAATGGAAGAAATTTCAAGAAATTTTTAATAAATTCTGCAGGCAAAAATACATCTAAGCTATGC
AAAAGAGATGGTTTCTGTCTTGGTATCATCCCAGGTTCTTATAACTTCCACTGGAAGATTTTAGAGTTGT
AGTGTTTACTATTAGAATGTTATTTAATCTCTAGTCAATGCCTCTTACTACAATGGAAGTGAATTTCCTC
TTTCTTTTCTTTTGAACAGCTGGGGGACGATAGGTCAGCTCTATTTTTATCAATAAACCTTCCAAACATT
TACAGATATCAAATAGCCCTTTATTTCTTTTTCTTGATGCAATAATATTAAGTTGTGCAACCTTTTCTCA
AAAGACCCATTTTCCTACCCATTTGTTGCTTTTCTTTAGACTGTCATCAGTTTTTCCATTGCCTTGAAAT
GTGGTGGCTAAAACTGGATGCCATGCCCTTTGAAGGGCTTGGCTCGTGTGGTTAGGGCTTTGTGAATGAG
TGATTTTTTGTTCTATGTAGCTCCTTGTGTTCTGTTGTTACCTCTCTGACCACAGCCTGCTTTCTCTTCA
TTGTAACTGCACTTCCCTGTGGGCTGCTTACCCATCTTGTTTTTAGTTCTCTCCTTTAATATACCTTCCA
TTTCAACAGCTTTTTGTTTCTGACACATGATTTGTATTGTTGTCTTAAAGTTCTATGTTCAGATATGAAA
GCCACACACCCTATGTAGCCAAGAAGTCCCTGTGCCCTTTGTTTTTAATGAAAAGGCACTTGAAGAACTG
AAGCCATAACAACAGTCTTCTGTGTTTATTGTTTCAGGATGGCAAAGACTACATTGTTCTTCCGATATCA
GAGACTTTGAGCATGGAAGAGGATTCTGGACTCTCTCTGCCTACCTCACCTGTTTCCTGTATGGAGGAGG
AGGAAGTATGTGACCCCAAATTCCATTATGACAACACAGCAGGAATCAGGTACTGTATATGGCCTAACAT
CCCCCGGGGGAGGGTGACTTCAAGGCCATCTCGGGAGGGGGATTGGAAGTGGAAGGAAGACCTTGTCTAA
GGCTGTTGCATCCCACTTCCACATAACCTTAGCCCTGAGGTTAACATAATGGGGAATGCTCCTGGAAGAG
GGCCTGGGTAGGTGTGCTTCCTCCCATCTGTAGCCCACGCTGCTGCCACAGCATTGCCTTTAAGAATTCC
AAGCCCTGCAGCTGCAATAGCTGGAATGCCACAGTTTGCTAATTTCCAGAATAAAGAGACGAGTTTTACA
AAGACATCTGCATTTAAATTATCCCCGTGTATGCTTTTATTAATGTGAATTAAATGGCTTAGGAGAGATT
CAGAAAGGAAGAGTTCTGTGCTTGCATGAGAACATGCTTATGGCTCTCTGGCAAGGATACAGAAAGCCAT
GGGTCTGTGTCCGGAATTAGACTGGACACTGCATCTCAGAAGCCCCTCCCACGTCTGATTTTCAGCATTT
TATTTGCATAATGGGATGTCTGGGCTTATTTAAAACACATGCACTGCAGTCCTTTCCTGATTTGCAGAGG
GGTTCTAAAGGCAGCTTTCTTTTTTCTCTCTCCCAGCACCTGTGCATAAGGAAAGAGTTGGTGTGGTTTT
CTACAATATGATATTAAAATTGCCCTTTACTAAGGCTGGGACTACTTCATTTTGCTTTGTTTCTTTCCTA
ACCCGTTTGGGTGTTTTCCTGCTTTAATGGAACCCCTGACAGCATGGGTCCAGCCTGCCAGCCCGAGTGT
GCCTGGGCTGCAGGGAGGGGCAGGGAGCTCTCTCATGTCCAGAACTTGGCCAGGTTGCCACATGGCAGGG
GATGCTAAGGAGAAACTCGTGGACAGTTTGCCCTCTAGAGTCGTGTGGGGCAGCAGAAACACTGATGGGA
AGGAAGAAAGCTTAGAAGCCAGCAAGACAGCTGACCGTTCCATTGAAGTCAAAAGCATTAGGCATATTTT
TAAAGAACTTTGCCGTATATTATCAGATGTTGCCCACATCATGACACTCAGAGTCAGGCAAGGTAGAAAC
AATGATCTTTTTTTTTGATGTATTATTGAACATGAGGCTCAGTTCTATTACCTGAGGGCAGTACAAACTT
GTAGTTAAAGATCAGGTATTAGAGTCAGATAGAAATGAGTAGGACCCCCAAGTCTGTCTTGTAGCAGCTG
TGCAACTTGGGGCAAATCATCTACCCTCTGCCTCAGTTTCTTTATCTGTGAAATGAGACAAGGTCAGTGG
TGCTGTTTGAAAATGGCTGTTTTGAGAGTTATAAGATATAATCTATTTCTAAGCACCTGGCCCTTGAAAG
CACTCAGTAAAAGATACCTATTAAGTGAGCTGCTTAAAATCACATCCTTGAGATGAATCCAGTTCCTCTG
ACCCCTAAGTCCATGTTGTTTCCTCCCATGCCAAGGAGGGCCCTCAGAGAGAAACAGTAATGAGATGAGA
CTACAATTCCACTCCTGTGTTTACACATTTCCAGTTCAAGTTGAGCTGGCCTTTTAGTGTGACAGTTGTT
CCCACACACCATTATTGCCTCCCCCTTTATCAGAAAGCCATTTGATCATGAACTACATTCCATGTGTTTT
CTGTGACCAAGTAGAGTGATGATCCGAGTCGGCAGCCTCCTGGCTCACCGGGTGCTTTGCATATGGTGCT
GAGCAGGAGAAGAAATCATGTTTGTGTAATGGAAGCACCAAATACGATGTTGGATATATAGAAGGGCTGC
TAACGTTTATCCCCAGAAGCGTGGACAAATGTGACACCACACTCCCAGCACAGGCCTGGCTCCTATTTTC
TGTCTGTGATTTTTGAATTGGTTTTTCCAGCCCAGTTTCTCTTTTATCCAGCCATAATTTGAAAAATAAA
ATGGAAATTGGAATCTTTTGTCTGCATCTCCTCTCCACCTCCTCCACCTTTTTTCCTTTCTATAAAATAA
AACTCACGGTCACATTTTAATCATCTGGTTTTGAAGAAAAGCAGATAGAGGCATTTGCACACGGCATGCT
TCATTCTGTTGCTCTCCTGGGGTTCTGTTTCTCTGGGGAGAATGAGTTGAGGCTGGGGTACTTCTCAGGG
AGCTTGTTCTATCCTCTTACGCATTTCTGGCCAAGTACAAAAGCTGAGCAGTCTTTCTCCTTCTAATTTT
CAATTCTATTGCATTATAAATAGAGTTGGACAGAGATATCACTGTGGGAGCTAGCTTCATGATTTGTTGC
CCCTTTAAACCATTTGAAAAATATTTACTTAGCATTTATTTAGAGAAAAGGCTGAGAAGTGTGTGGGGGA
GGGACCACTCATGTCTAGACTTAGCTTTGCCTCTAATTTCCCCTGTGGACCAGCTCTGGCCTCAAGTTTG
CATGCTTCCTGCAAGAAAACACATACTTGCTGGGCTCATCTTTCTTTGAGGGCAGTTTGGGGACCATCGG
CAATTGCTCTGTCATTTTCCCTGGGAGTTTCACCTCACACATCAAGCAGCTTATCAAAAATTTCTTTGCA
GTTCTCTCTTAGAGAAAGGTTTTGGTACATACCATTTTCTTCATTTTGTAATTGTTAGGGATGATTAAAT
GGCCCTTGTAGATTGATGCTTGGGGCAGCCTGCTAGCTAGGTATTCCTGAGTTTGGCTCTACCATTAGAC
TGTTTGCAGTGGGACTGTCCTTTCTGCACTTTTTGTCTGTTTCATACCCCGTACTTACACCCCTGACCCT
GCTACTGCATGATCAGTGCATGCATGACAAGAGAACAGTGCTGTGCACATACTGGGTGCTTAATAATGGC
TTGAACAATTGTGTCTGCTGTTTTCTTCTTTCTTTTCCCTCCTGATACTCTTCCAAGGGAGTCTGTATGG
AGTAGAGTAAAACAAAACAAAAACTTCACATGGGCTTTAGTGTCTGAAGGCCTAAGTTTGAGTCCCAGTT
CTACCTTTTATTAGCCATTTTCTCCCTAATCCTTGACTCCCTCATCTCCAAAGGGGAAATAGTTAAAAGA
CCTGTTTCTCCGTCTTAGGAGAAACAGATGCACCATTGTCTGTGAAAATGCTTTGTCAATCATGAGAGGA
TCATGCCATTTAAAAAATTACTGGATTAAGAATTTAAGGAGCTGTCCTTTCTAAGGCAGCTGAATTATTG
TCCAAACTCGCCAACCCTAGTTGATTCTATCCCCTAGATATCTCTAGAATGAGCCCATGTCTCCAAACCT
CATGGGCATTCCCTTTTTCTAGCCAAGCTGCCTTTCTTTCTCCTGAAGAAGTGCAGTATTTGTCTCTTGG
GTCTTATGCCTCTAGTCTTATTCTTTTCAATCCAGAGTCAATTCTCTAAAGGGCATATCTGATCTTGTCA
ATCCCATGCCTAAAATCCTTCAGTGGCTCTTCATTGCCCTCAAAATAATAATCCAAACATTCCAGTTATG
TGATTTTGGATAAGTTCCTCAAATTTTCTATGCCTTGGTTTCCTCATCTGAAGAGTTGGGATAGTAATAC
TCACCCCTAGAGAGGTACCGTGGTGAACACATCATGAGATGCTGCTTAGACAGCTTCTGGCACAGTGTCA
GGCTTGCGGCAGATTATCAGTGAGGGCTTCCTGAACAAGTGAATGCAGGAATGATTGACTACGGTACCAG
TAGTGTTTGACAACTGTTACTTTTAGGGGTTGGACTTAGAAAGTAGGCTTTGCTTGCACCCTGTGTATCA
TATCCTCTTAACTTGTGGAGTTTCCTGAGTGAGGATGTCACCGGAAAATCTCATTCTCTCCTCTCTCTAT
AGGGAGGAACCAGCCTCTTGGGGTAGGGGAGAGAGAATTAATTTCCATTCTTCTCCTTTGGCCCAAGGTC
TATGCAGCATGTTCCAGAAGTCTGCTTGTAGTGGGAAGTAGGCTGGTATAGGAATGAAGAATGTATTTTC
TGTCTCGGTGGGCCCTTCCAGTGAATAGGACTTCCCTTCCCTCCACTTGGGCTGTAAGTGATTTTGATAG
CATCAACTAGACTCACCCAAAGCCACACGGCCGGGAAGGAGCATTCTCAAGAAGGAGAGGATCTGTTGTT
CAACAAGTCTTATTCTTTGGACTCCTGAAGGAAGCTTTGGAAGTCAAAGGAGAAAAATGAGCTTTGTTTG
AAGAGGGCATTATTCTTCCTAAGAGCAATAAGCCCAACATTCTCTATGTCATTCATCTTCCCAACATCCC
TGTGAGCTGGGGAGGGAGTGCTACTGCCAACACATCTTATAGATGGGACAAGAGGGTCACAGAAATATTC
ATGACTTTCTCAAGTTTCTGCAGTCAGTGGTAGACTCTGAAATAGGCAAAATATCTTGTTATTCTCAAAC
CACTGCTCTTTCCTGAGACAGCAACTCTGGGGGCGAAAACGAGGGGACAGTGAGACTCAGCCCACCTTCT
CTTTGCACACCAAGCCTCTGTTACATGGAGGAGGAAGAGGTTGTCTTCAAATCACTGCTGGGTTCAGTAT
CCTTTAAGGAGACCTTCAGATGTTTCCTCTGCCTATCTTTCATTGAATGGTTGCTCTGTGAGCATTATCC
AGAAAAACTTTCCCAGGAGATGGCCAGACAGATGTGAAACACTCAGTAATATATCCAGAGCTCGATGGAG
GAATCCCATGCAATCAGGAAGCCAAGTAGAAGGCAGTTGATCACTCCATCTGCTGTTGTTGTCTTTAGTC
CAGAACTGGACCTCAGAAGTAGGATTCAAAAGAACAGGCTCATCGAGACTCCTCAGTTATATTATACTTT
TAAATGTACTTTCTCAGGAAATTAAGCCTTCCATGTGTGCTAGCAGAGAAAGATTTTTATTTTGTTTTGT
TTTTCTAAAGGATGTTTTGAAGGTTGCTATTAAGTTTGTGGTTGAAAGATAATGAACTTAGGTAGCCGAT
CTGCAGTCAAATATACCACCACTAAAATATAAATATTTGTTCTTTTGCAGTCAGTATCTGCAGAACAGTA
AGCGAAAGAGCCGGCCTGTGAGTGTAAAAACATTTGAAGATATCCCGTTAGAAGAACCAGAAGTAAAAGT
AATCCCAGATGTAAGTACGTCTTTTAAAAATAGTCTTAGAAATAATACAAAGGATGAAACACTAGCTAGA
TAAATATTAGCCTAAGCATTAAAGTTTTGGAGCCTCATTAGAAGGCTGCCCTCGAGTGTGTGTATCATGG
GGTCATTATGGAGATGGAACTTTGTTTTTTTCATAAGTAAAGCCCTTGGTCCAAGGTTCAAGACAGTGTA
GCTTTCTGACCAATTTCACTAAAGTGCAAGTAGTGTCATAGTGAAGACAGCGATGGTAACAGGCATTCTC
AGCTGCTGATTTGTAAATTTTCTCTTCTCCCTGGCCTGTGTCTACTCATAGGAAGCAGTTGCTTCCTTTT
GTAGCTTGGACAATTTGTGGCTATGATACCTTTATGTTCTTCCACAGGACCTTATTTGATAGACATGATA
GATGGGTTGAGAAATCAGCTTAATTAAATAGTTGGTCATTTTATATGCTCAATTAACTGTGCCATCTCAT
TGTCTCTTAAAAAGGACAACCAGACGGACAGTGGTATGGTTCTTGCCTCAGAAGAGCTGAAAACTTTGGA
AGACAGAACCAAATTATCTCCATCTTTTGGGTAAGACTCAGCCATATTAAAAAGACAAATTTCAATAGGA
ATTTTTGGAAGGAACTTAGGACTTTCAGTGTAAGTGCAGAATTTTCCCTATGGGGTCTTTGTTGGTTGGA
GAAATTAGCATCAATTTAACAAATAAAGAATGGAAACTAACCACACAATAAAATTAAGTGATAAATCTAA
AAATAATCTGAAATAAATTAGAGAATTTGGTCAATTTTTATGAGAATTCATGAATACTAGGGAATTTCTG
TGTATATTTACTGTGGTCAGTAATGGCTAAATGAAAAAGGTGATTGGATGTGATCCGTAAAGCTGTCAAT
ATGATTACAATCTTTGTGGACTCTGAAGAATTTTTAAGTCTGTATACAAATGGGTGCATCTGTGCTTAAG
AAGTATGATATATAAATAAGCCAATATCTATTTGTTTGAGACATTTAAATATTATTGTCTGAATTCGAAG
TATTTCATTGTGAGAAAAGTATTAAAATTAGTTTTAAATATAATCTCCCTTCTATGGCTCAGTAGGAATT
TGTAGGTGTCTTGAATACGTGTACGTTCTCTTAACATAACAAATCAATGAAAATCTATATTTATAAGAAT
AATAGAATAAGTGTAGTTATGTATTTGCTGGAGTTTATTTGCTAGAGTATTCTTACCTAAAGGTAAGAAT
AGAGGAGGTTTTGATCTGCTTATAATCTTTTATATAAAATGGGAATACTCATGGGTTTTTGAATAATGCT
CATACCAAAAAGAAAACAAACAAAAAAAACCCCAACATATTAAAAGGTGCCATTGTGCTATTTTATTGTT
TTCTTTAAGGCCCAAGGTAAGAAATTGTGAAAGTCAATGATATGTTTCATTCATTGATTCAAAAAATGTT
TATTCGGCAAGTATCATGTGCAGAGCACCATGCCATTGCTTGAGACACCTACATTAGTTTTGTTGGGGTT
GAATTGAAAGAAAAAATTGTATTTCTCATTATTTGAAGTAACTTTTAAACTATGTATAAACACGAGTTAC
TAAAATTCCCTTTTGCAGTTTTAACATGAAGAAGTTGGGGAAAACACCTATTACCGGGAAAAAACACCTT
AGAATGGCTTGTGAAAGTGTAAATCCTGAAGTTTTAGATCAACACAGCCTGCATTTCTAGGCTTTGACAT
GATTACCGTCTGTCAGGATTCCATGCCATTGAAAACATTTTCTAGTTGCTGCTGAGTGACAGGGGTTCTC
AGTCCTTCCAAGGAATGTGGTTTTGATGAGTAAAAAGCAGCGTTTGATATGTCTGGCTTGACTGCACACA
TGCTTCAAGTTATTAAAGTTTAAAGTTGCTCAAGAGCTTTATTACAACCATACACATGCCCCGTAATTCC
CAAATTGCCACAATAGGAAAAGCACAAGTGAAATTTAAGAACATCCCAATTTCCTTGAATATCATGCAAG
TGGCCCTTTGGCGCCTGTCACTGTATACAAATTTGTCAATCTGCGAGGCCATAAACATGTTCCATCAGTT
GGGGCCTTTGCATAACTCGAGAGAACTGCCTTTCATCTCATTTGAGGCTTGAAAGACTTGGACCTGAGTA
AGAGGACTTATCTGCAACTACTAATTCATGCGAGTACCTGAAAATAGACCTTGTCCCTGTAAACCTGCTA
TGCTGATTAACAACTGGGAGAGATACGGGGCTGCGGTCTCCAGGGAGATGGCAGCCATATGGAGTTGGGA
ATGGGGTGAGGGTAAAAAGCAAAAGAATTGTCTTCTCTCTGCCAACTCCTTTGTTTGCCATTTCTTCTGC
AGTGGAATGGTGCCCAGCAAAAGCAGGGAGTCTGTGGCATCTGAAGGCTCAAACCAGACAAGCGGCTACC
AGTCCGGATATCACTCCGATGACACAGACACCACCGTGTACTCCAGTGAGGAAGCAGAACTTTTAAAGCT
GATAGAGATTGGAGTGCAAACCGGTAGCACAGCCCAGATTCTCCAGCCTGACTCGGGGACCACACTGAGC
TCTCCTCCTGTTTAAAAGGAAGCATCCACACCCCCAACTCCTGGACATCACATGAGAGGTGCTGCTCAGA
TTTTCAAGTGTTGTTCTTTCCACCAGCAGGAAGTAGCCGCATTTGATTTTCATTTCGACAACAGAAAAAG
GACCTCGGACTGCAGGGAGCCAGTCTTCTAGGCATATCCTGGAAGAGGCTTGTGACCCAAGAATGTGTCT
GTGTCTTCTCCCAGTGTTGACCTGATCCTCTTTTTCATTCATTTAAAAAGCATTTATCATGCCCCCTGCT
GCGGGTCTCACCATGGGTTTAGAACAAAGACGTTCAAGAAATGGCCCCATCCTCAAAGAAGTAGCAGTAC
CTGGGGAGCTGACACTTCTGTAAAACTAGAAGATAAACCAGGCAATGTAAGTGTTCGAGGTGTTGAAGAT
GGGAAGGATTTGCAGGGCTGAGTCTATCCAAGAGGCTTTGTTTAGGACGTGGGTCCCAAGCCAAGCCTTA
AGTGTGGAATTCGGATTGATAGAAAGGAAGACTAACGTTACCTTGCTTTGGAGAGTACTGGAGCCTGCAA
ATGCATTGTGTTTGCTCTGGTGGAGGTGGGCATGGGGTCTGTTCTGAAATGTAAAGGGTTCAGACGGGGT
TTCTGGTTTTAGAAGGTTGCGTGTTCTTCGAGTTGGGCTAAAGTAGAGTTCGTTGTGCTGTTTCTGACTC
CTAATGAGAGTTCCTTCCAGACCGTTACGTGTCTCCTGGCCAAGCCCCAGGAAGGAAATGATGCAGCTCT
GGCTCCTTGTCTCCCAGGCTGATCCTTTATTCAGAATACCACAAAGAAAGGACATTCAGCTCAAGGCTCC
CTGCCGTGTTGAAGAGTTCTGACTGCACAAACCAGCTTCTGGTTTCTTCTGGAATGAATACCCTCATATC
TGTCCTGATGTGATATGTCTGAGACTGAATGCGGGAGGTTCAATGTGAAGCTGTGTGTGGTGTCAAAGTT
TCAGGAAGGATTTTACCCTTTTGTTCTTCCCCCTGTCCCCAACCCACTCTCACCCCGCAACCCATCAGTA
TTTTAGTTATTTGGCCTCTACTCCAGTAAACCTGATTGGGTTTGTTCACTCTCTGAATGATTATTAGCCA
GACTTCAAAATTATTTTATAGCCCAAATTATAACATCTATTGTATTATTTAGACTTTTAACATATAGAGC
TATTTCTACTGATTTTTGCCCTTGTTCTGTCCTTTTTTTCAAAAAAGAAAATGTGTTTTTTGTTTGGTAC
CATAGTGTGAAATGCTGGGAACAATGACTATAAGACATGCTATGGCACATATATTTATAGTCTGTTTATG
TAGAAACAAATGTAATATATTAAAGCCTTATATATAATGAACTTTGTACTATTCACATTTTGTATCAGTA
TTATGTAGCATAACAAAGGTCATAATGCTTTCAGCAATTGATGTCATTTTATTAAAGAACATTGAAAAAC
TTGAAGGAATCCCTTTGCAAGGTTGCATTACTGTACCCATCATTTCTAAAATGGAAGAGGGGGTGGCTGG
GCACAGTGGCCGACACCTAAAAACCCAGCACTTTGGGGGGCCAAGGTGGGAGGATCGCTTGAGCCCAGGA
GTTCAAGACCAGTCTGGCCAACATGGTCAGATTCCATCTCAAAGAAAAAAGGTAAAAATAAAATAAAATG
GAGAAGAAGGAATCAGA
SEQ ID NO: 2
>gi|195546779|ref|NM_002253.2| Homo sapiens kinase insert do-
main receptor (a type III receptor tyrosine kinase) (KDR), mRNA
ACTGAGTCCCGGGACCCCGGGAGAGCGGTCAATGTGTGGTCGCTGCGTTTCCTCTGCCTGCGCCGGGCAT
CACTTGCGCGCCGCAGAAAGTCCGTCTGGCAGCCTGGATATCCTCTCCTACCGGCACCCGCAGACGCCCC
TGCAGCCGCGGTCGGCGCCCGGGCTCCCTAGCCCTGTGCGCTCAACTGTCCTGCGCTGCGGGGTGCCGCG
AGTTCCACCTCCGCGCCTCCTTCTCTAGACAGGCGCTGGGAGAAAGAACCGGCTCCCGAGTTCTGGGCAT
TTCGCCCGGCTCGAGGTGCAGGATGCAGAGCAAGGTGCTGCTGGCCGTCGCCCTGTGGCTCTGCGTGGAG
ACCCGGGCCGCCTCTGTGGGTTTGCCTAGTGTTTCTCTTGATCTGCCCAGGCTCAGCATACAAAAAGACA
TACTTACAATTAAGGCTAATACAACTCTTCAAATTACTTGCAGGGGACAGAGGGACTTGGACTGGCTTTG
GCCCAATAATCAGAGTGGCAGTGAGCAAAGGGTGGAGGTGACTGAGTGCAGCGATGGCCTCTTCTGTAAG
ACACTCACAATTCCAAAAGTGATCGGAAATGACACTGGAGCCTACAAGTGCTTCTACCGGGAAACTGACT
TGGCCTCGGTCATTTATGTCTATGTTCAAGATTACAGATCTCCATTTATTGCTTCTGTTAGTGACCAACA
TGGAGTCGTGTACATTACTGAGAACAAAAACAAAACTGTGGTGATTCCATGTCTCGGGTCCATTTCAAAT
CTCAACGTGTCACTTTGTGCAAGATACCCAGAAAAGAGATTTGTTCCTGATGGTAACAGAATTTCCTGGG
ACAGCAAGAAGGGCTTTACTATTCCCAGCTACATGATCAGCTATGCTGGCATGGTCTTCTGTGAAGCAAA
AATTAATGATGAAAGTTACCAGTCTATTATGTACATAGTTGTCGTTGTAGGGTATAGGATTTATGATGTG
GTTCTGAGTCCGTCTCATGGAATTGAACTATCTGTTGGAGAAAAGCTTGTCTTAAATTGTACAGCAAGAA
CTGAACTAAATGTGGGGATTGACTTCAACTGGGAATACCCTTCTTCGAAGCATCAGCATAAGAAACTTGT
AAACCGAGACCTAAAAACCCAGTCTGGGAGTGAGATGAAGAAATTTTTGAGCACCTTAACTATAGATGGT
GTAACCCGGAGTGACCAAGGATTGTACACCTGTGCAGCATCCAGTGGGCTGATGACCAAGAAGAACAGCA
CATTTGTCAGGGTCCATGAAAAACCTTTTGTTGCTTTTGGAAGTGGCATGGAATCTCTGGTGGAAGCCAC
GGTGGGGGAGCGTGTCAGAATCCCTGCGAAGTACCTTGGTTACCCACCCCCAGAAATAAAATGGTATAAA
AATGGAATACCCCTTGAGTCCAATCACACAATTAAAGCGGGGCATGTACTGACGATTATGGAAGTGAGTG
AAAGAGACACAGGAAATTACACTGTCATCCTTACCAATCCCATTTCAAAGGAGAAGCAGAGCCATGTGGT
CTCTCTGGTTGTGTATGTCCCACCCCAGATTGGTGAGAAATCTCTAATCTCTCCTGTGGATTCCTACCAG
TACGGCACCACTCAAACGCTGACATGTACGGTCTATGCCATTCCTCCCCCGCATCACATCCACTGGTATT
GGCAGTTGGAGGAAGAGTGCGCCAACGAGCCCAGCCAAGCTGTCTCAGTGACAAACCCATACCCTTGTGA
AGAATGGAGAAGTGTGGAGGACTTCCAGGGAGGAAATAAAATTGAAGTTAATAAAAATCAATTTGCTCTA
ATTGAAGGAAAAAACAAAACTGTAAGTACCCTTGTTATCCAAGCGGCAAATGTGTCAGCTTTGTACAAAT
GTGAAGCGGTCAACAAAGTCGGGAGAGGAGAGAGGGTGATCTCCTTCCACGTGACCAGGGGTCCTGAAAT
TACTTTGCAACCTGACATGCAGCCCACTGAGCAGGAGAGCGTGTCTTTGTGGTGCACTGCAGACAGATCT
ACGTTTGAGAACCTCACATGGTACAAGCTTGGCCCACAGCCTCTGCCAATCCATGTGGGAGAGTTGCCCA
CACCTGTTTGCAAGAACTTGGATACTCTTTGGAAATTGAATGCCACCATGTTCTCTAATAGCACAAATGA
CATTTTGATCATGGAGCTTAAGAATGCATCCTTGCAGGACCAAGGAGACTATGTCTGCCTTGCTCAAGAC
AGGAAGACCAAGAAAAGACATTGCGTGGTCAGGCAGCTCACAGTCCTAGAGCGTGTGGCACCCACGATCA
CAGGAAACCTGGAGAATCAGACGACAAGTATTGGGGAAAGCATCGAAGTCTCATGCACGGCATCTGGGAA
TCCCCCTCCACAGATCATGTGGTTTAAAGATAATGAGACCCTTGTAGAAGACTCAGGCATTGTATTGAAG
GATGGGAACCGGAACCTCACTATCCGCAGAGTGAGGAAGGAGGACGAAGGCCTCTACACCTGCCAGGCAT
GCAGTGTTCTTGGCTGTGCAAAAGTGGAGGCATTTTTCATAATAGAAGGTGCCCAGGAAAAGACGAACTT
GGAAATCATTATTCTAGTAGGCACGGCGGTGATTGCCATGTTCTTCTGGCTACTTCTTGTCATCATCCTA
CGGACCGTTAAGCGGGCCAATGGAGGGGAACTGAAGACAGGCTACTTGTCCATCGTCATGGATCCAGATG
AACTCCCATTGGATGAACATTGTGAACGACTGCCTTATGATGCCAGCAAATGGGAATTCCCCAGAGACCG
GCTGAAGCTAGGTAAGCCTCTTGGCCGTGGTGCCTTTGGCCAAGTGATTGAAGCAGATGCCTTTGGAATT
GACAAGACAGCAACTTGCAGGACAGTAGCAGTCAAAATGTTGAAAGAAGGAGCAACACACAGTGAGCATC
GAGCTCTCATGTCTGAACTCAAGATCCTCATTCATATTGGTCACCATCTCAATGTGGTCAACCTTCTAGG
TGCCTGTACCAAGCCAGGAGGGCCACTCATGGTGATTGTGGAATTCTGCAAATTTGGAAACCTGTCCACT
TACCTGAGGAGCAAGAGAAATGAATTTGTCCCCTACAAGACCAAAGGGGCACGATTCCGTCAAGGGAAAG
ACTACGTTGGAGCAATCCCTGTGGATCTGAAACGGCGCTTGGACAGCATCACCAGTAGCCAGAGCTCAGC
CAGCTCTGGATTTGTGGAGGAGAAGTCCCTCAGTGATGTAGAAGAAGAGGAAGCTCCTGAAGATCTGTAT
AAGGACTTCCTGACCTTGGAGCATCTCATCTGTTACAGCTTCCAAGTGGCTAAGGGCATGGAGTTCTTGG
CATCGCGAAAGTGTATCCACAGGGACCTGGCGGCACGAAATATCCTCTTATCGGAGAAGAACGTGGTTAA
AATCTGTGACTTTGGCTTGGCCCGGGATATTTATAAAGATCCAGATTATGTCAGAAAAGGAGATGCTCGC
CTCCCTTTGAAATGGATGGCCCCAGAAACAATTTTTGACAGAGTGTACACAATCCAGAGTGACGTCTGGT
CTTTTGGTGTTTTGCTGTGGGAAATATTTTCCTTAGGTGCTTCTCCATATCCTGGGGTAAAGATTGATGA
AGAATTTTGTAGGCGATTGAAAGAAGGAACTAGAATGAGGGCCCCTGATTATACTACACCAGAAATGTAC
CAGACCATGCTGGACTGCTGGCACGGGGAGCCCAGTCAGAGACCCACGTTTTCAGAGTTGGTGGAACATT
TGGGAAATCTCTTGCAAGCTAATGCTCAGCAGGATGGCAAAGACTACATTGTTCTTCCGATATCAGAGAC
TTTGAGCATGGAAGAGGATTCTGGACTCTCTCTGCCTACCTCACCTGTTTCCTGTATGGAGGAGGAGGAA
GTATGTGACCCCAAATTCCATTATGACAACACAGCAGGAATCAGTCAGTATCTGCAGAACAGTAAGCGAA
AGAGCCGGCCTGTGAGTGTAAAAACATTTGAAGATATCCCGTTAGAAGAACCAGAAGTAAAAGTAATCCC
AGATGACAACCAGACGGACAGTGGTATGGTTCTTGCCTCAGAAGAGCTGAAAACTTTGGAAGACAGAACC
AAATTATCTCCATCTTTTGGTGGAATGGTGCCCAGCAAAAGCAGGGAGTCTGTGGCATCTGAAGGCTCAA
ACCAGACAAGCGGCTACCAGTCCGGATATCACTCCGATGACACAGACACCACCGTGTACTCCAGTGAGGA
AGCAGAACTTTTAAAGCTGATAGAGATTGGAGTGCAAACCGGTAGCACAGCCCAGATTCTCCAGCCTGAC
TCGGGGACCACACTGAGCTCTCCTCCTGTTTAAAAGGAAGCATCCACACCCCCAACTCCTGGACATCACA
TGAGAGGTGCTGCTCAGATTTTCAAGTGTTGTTCTTTCCACCAGCAGGAAGTAGCCGCATTTGATTTTCA
TTTCGACAACAGAAAAAGGACCTCGGACTGCAGGGAGCCAGTCTTCTAGGCATATCCTGGAAGAGGCTTG
TGACCCAAGAATGTGTCTGTGTCTTCTCCCAGTGTTGACCTGATCCTCTTTTTCATTCATTTAAAAAGCA
TTTATCATGCCCCCTGCTGCGGGTCTCACCATGGGTTTAGAACAAAGACGTTCAAGAAATGGCCCCATCC
TCAAAGAAGTAGCAGTACCTGGGGAGCTGACACTTCTGTAAAACTAGAAGATAAACCAGGCAATGTAAGT
GTTCGAGGTGTTGAAGATGGGAAGGATTTGCAGGGCTGAGTCTATCCAAGAGGCTTTGTTTAGGACGTGG
GTCCCAAGCCAAGCCTTAAGTGTGGAATTCGGATTGATAGAAAGGAAGACTAACGTTACCTTGCTTTGGA
GAGTACTGGAGCCTGCAAATGCATTGTGTTTGCTCTGGTGGAGGTGGGCATGGGGTCTGTTCTGAAATGT
AAAGGGTTCAGACGGGGTTTCTGGTTTTAGAAGGTTGCGTGTTCTTCGAGTTGGGCTAAAGTAGAGTTCG
TTGTGCTGTTTCTGACTCCTAATGAGAGTTCCTTCCAGACCGTTACGTGTCTCCTGGCCAAGCCCCAGGA
AGGAAATGATGCAGCTCTGGCTCCTTGTCTCCCAGGCTGATCCTTTATTCAGAATACCACAAAGAAAGGA
CATTCAGCTCAAGGCTCCCTGCCGTGTTGAAGAGTTCTGACTGCACAAACCAGCTTCTGGTTTCTTCTGG
AATGAATACCCTCATATCTGTCCTGATGTGATATGTCTGAGACTGAATGCGGGAGGTTCAATGTGAAGCT
GTGTGTGGTGTCAAAGTTTCAGGAAGGATTTTACCCTTTTGTTCTTCCCCCTGTCCCCAACCCACTCTCA
CCCCGCAACCCATCAGTATTTTAGTTATTTGGCCTCTACTCCAGTAAACCTGATTGGGTTTGTTCACTCT
CTGAATGATTATTAGCCAGACTTCAAAATTATTTTATAGCCCAAATTATAACATCTATTGTATTATTTAG
ACTTTTAACATATAGAGCTATTTCTACTGATTTTTGCCCTTGTTCTGTCCTTTTTTTCAAAAAAGAAAAT
GTGTTTTTTGTTTGGTACCATAGTGTGAAATGCTGGGAACAATGACTATAAGACATGCTATGGCACATAT
ATTTATAGTCTGTTTATGTAGAAACAAATGTAATATATTAAAGCCTTATATATAATGAACTTTGTACTAT
TCACATTTTGTATCAGTATTATGTAGCATAACAAAGGTCATAATGCTTTCAGCAATTGATGTCATTTTAT
TAAAGAACATTGAAAAACTTGAAGGAATCCCTTTGCAAGGTTGCATTACTGTACCCATCATTTCTAAAAT
GGAAGAGGGGGTGGCTGGGCACAGTGGCCGACACCTAAAAACCCAGCACTTTGGGGGGCCAAGGTGGGAG
GATCGCTTGAGCCCAGGAGTTCAAGACCAGTCTGGCCAACATGGTCAGATTCCATCTCAAAGAAAAAAGG
TAAAAATAAAATAAAATGGAGAAGAAGGAATCAGA
SEQ ID NO: 3
>gi|568815592:43770209-43786487 Homo sapiens chromosome 6,
GRCh38 Primary Assembly
TCGCGGAGGCTTGGGGCAGCCGGGTAGCTCGGAGGTCGTGGCGCTGGGGGCTAGCACCAGCGCTCTGTCG
GGAGGCGCAGCGGTTAGGTGGACCGGTCAGCGGACTCACCGGCCAGGGCGCTCGGTGCTGGAATTTGATA
TTCATTGATCCGGGTTTTATCCCTCTTCTTTTTTCTTAAACATTTTTTTTTAAAACTGTATTGTTTCTCG
TTTTAATTTATTTTTGCTTGCCATTCCCCACTTGAATCGGGCCGACGGCTTGGGGAGATTGCTCTACTTC
CCCAAATCACTGTGGATTTTGGAAACCAGCAGAAAGAGGAAAGAGGTAGCAAGAGCTCCAGAGAGAAGTC
GAGGAAGAGAGAGACGGGGTCAGAGAGAGCGCGCGGGCGTGCGAGCAGCGAAAGCGACAGGGGCAAAGTG
AGTGACCTGCTTTTGGGGGTGACCGCCGGAGCGCGGCGTGAGCCCTCCCCCTTGGGATCCCGCAGCTGAC
CAGTCGCGCTGACGGACAGACAGACAGACACCGCCCCCAGCCCCAGCTACCACCTCCTCCCCGGCCGGCG
GCGGACAGTGGACGCGGCGGCGAGCCGCGGGCAGGGGCCGGAGCCCGCGCCCGGAGGCGGGGTGGAGGGG
GTCGGGGCTCGCGGCGTCGCACTGAAACTTTTCGTCCAACTTCTGGGCTGTTCTCGCTTCGGAGGAGCCG
TGGTCCGCGCGGGGGAAGCCGAGCCGAGCGGAGCCGCGAGAAGTGCTAGCTCGGGCCGGGAGGAGCCGCA
GCCGGAGGAGGGGGAGGAGGAAGAAGAGAAGGAAGAGGAGAGGGGGCCGCAGTGGCGACTCGGCGCTCGG
AAGCCGGGCTCATGGACGGGTGAGGCGGCGGTGTGCGCAGACAGTGCTCCAGCCGCGCGCGCTCCCCAGG
CCCTGGCCCGGGCCTCGGGCCGGGGAGGAAGAGTAGCTCGCCGAGGCGCCGAGGAGAGCGGGCCGCCCCA
CAGCCCGAGCCGGAGAGGGAGCGCGAGCCGCGCCGGCCCCGGTCGGGCCTCCGAAACCATGAACTTTCTG
CTGTCTTGGGTGCATTGGAGCCTTGCCTTGCTGCTCTACCTCCACCATGCCAAGGTAAGCGGTCGTGCCC
TGCTGGCGCCGCGGGCCGCTGCGAGCGCCTCTCCCGGCTGGGGACGTGCGTGCGAGCGCGCGCGTGGGGG
CTCCGTGCCCCACGCGGGTCCATGGGCACCAGGCGTGCGGCGTCCCCCTCTGTCGTCTTAGGTGCAGGGG
GAGGGGGCGCGCGCGCTAGGTGGGAGGGTACCCGGAGAGAGGCTCACCGCCCACGCGGGCCCTGCCCACC
CACCGGAGTCACCGCACGTACGATCTGGGCCGACCAGCCGAGGGCGGGAGCCGGAGGAGGAGGCCGAGGG
GGCTGGGCTTGCGTTGCCGCTGCCGGCTGAAGTTTGCTCCCGGCCGCTGGTCCCGGACGAACTGGAAGTC
TGAGCAGCGGGGGCGGGAGCCAGAGACCAGTGGGCAGGGGGTGCTCGGACCTTGGACCGCGGGAGGGCAG
AGAGCGTGGAGGGGGCAGGGCGCAGGAGGGAGAGGGGGCTTGCTGTCACTGCCACTCGGTCTCTTCAGCC
CTCGCCGCGAGTTTGGGAAAAGTTTTGGGGTGGATTGCTGCGGGGACCCCCCCTCCCTGCTGGGCCACCT
GCGCCGCGCCAACCCCGCCCGTCCCCGCTCGCGTCCCGCTCGGTGCCCGCCCTCCCCCGCCCGGCCGGGT
GCGCGCGGCGCGGAGCCGATTACATCAGCCCGGGCCTGGCCGGCCGCGTGTTCCCGGAGCCTCGGCTGCC
CGAATGGGGAGCCCAGAGTGGCGAGCGGCACCCCTCCCCCCGCCAGCCCTCCGCGGGAAGGTGACCTCTC
GAGGTAGCCCCAGCCCGGGGATCCAGAGAACCATCCCTACCCCTTCCTACTGTCTCCAGACCCTACCTCT
GCCCAGTGCTAGGAGGAATTTCCTGACGCCCCTTCTCTTCACCCATTTCCTTTTTAGCCTGGAGAGAAGC
CCCTGTCACCCCGCTTATTTTCATTTCTCTCTGCGGAGAAGATCCATCTAACCCCTTTCTGGCCCCAGAG
TCCAGGGAAAGGATGATCACTGTCAGAAGTCGTGGCGCGGGAGCCCACTGGGCGCTTTGTCACATTCCAC
CGAAAGTCCCGACTTGGTGACAGTGTGCTTCCCTTCCCTCGCCAACAGTTCCGAGTGAGCTGTGCTTTAG
CTCTCGTGGGGGTGGGTCAAGGGAGGATTTGAAGAGTCATTGCCCCACTTTACCCTTTTGGAGAAATGGC
TTGAAATTTGCTGTGACACGGGCAGCATGGGAATAGTCCTTCCTGAACCCTGGAAAGGAGCTCCTGCCAG
CCTTGCACACACTTTGTCCTGGTGAAAGGCAGCCCTGGAGCAGGTGTTTTTTTGGAACTCCAAACCTGCC
CACCCAACTTGCTTCTGAAAGGGACTCTAAAGGGTCCCTTTCCGCTCCTCTCTGACGCCTTCCCTCAGCC
AGAATTCCCTTGGAGAGGAGGCAAGAGGAAAGCCATGGACAGGGGTCGCTGCTAACACCGCAAGTTCCTC
AGACCCTGGCACAAAGGCCTTGGCTACAGGCCTCCAAGTAGGGAGGAGGGGGAGGAGTGGCTGCCTGGCC
ACAGTGTGACCTTCAGAGGCCCCCAGAGAAGGACACCTGGCCCCTGCCTGCCTAGAACCGCCCCTCCTGT
GCTCCCTGGCCTTGGAAGGGGTATGAAATTTCCGTCCCCTTTCCTCCTTGGGGCCCAGGAGGAGTGGAGG
GTCCCGGGAGAATATTGTCAGGGGGAAGGCAGGGGGTGTCATGGGAATGGGTGAGGGGGCTGAGGTGCAG
AATCCAGGGGGTCCCTGCAGGAGCCGCAGTGGTAAGCTGTCCAGCTGGAAGCCTGGTAACTGTTGTTTTC
TCTTGAGAGGGGCTTCCTGTGACCTTGGCTGTCTCTGGGAGCAGGGCTGGGGTACCTGAGTGGGGTGCAT
TTGGGGTGTGTGGGAAGGAGAGGGAAAGAAAGATGGACAGTGGGACTCTCCCCTAGCAGGGTCTGGTGTT
CCGTAGGCTAGAGTGCCCCTCTGCTCTGCGAGTGCTGGGCGGGAGGGGAGTTGGTGAGAGCTGGAGACCC
CCAGGAAGGGCTGGCAGAAGCCTTTCCTTTTGGGTGCTGTCAGGTCCGCATGTCTTGGCGTGTTGACCTT
CACAGCTTCTGGCGAGGGGAGGAATGATCTGATGCGGGTGGGGAGGGTTAGAGGAGGCCTCAGGCCTAAG
GTGGTGCAGGGGGCCCCCTAGGGGCTGGGCAGTGCCAAGGCATAAAAGCCTTCCCTGGTCCCTGGTGGCA
TTTGAAGGTGCCCAGGTGAGAGGGGCTTGGCACCTCCTCACCCTGGGAGGGAGAAGAAACCAGGGAACAG
GTAGGAGTGGGAGACAGGTGAGGCTTTGGAAATCTATTGAGGCTCTGGAGAGATTTGTGTAGAGAGGAAA
ATGTGGTTCTCCCCCAGGGTCTCCTCCTGGGTTTTTACCCTCTAAGCAACCTGTGGGCATGCTGGGTTAT
TCCTAAGGACTAGAAGAGCTTGGATGGGGGAGGGTGGTTGGTGCCCTTCGGTCCTCGGCACCCCCCTCCG
TCTCCAACACCAGCTCACCCTGGTATTTGTCATGTCAGCAGGAGAAGGTCACCATGTTGTTTTTCTCGCC
CCTAGTCCTTCCTTCCTGCCCCAGTCCAAATTTGTCCTCCTATTTGACCTTAATACTTACCATGGCTTTG
GACCAGGGAACTAGGGGGATAGTGAGAGCAGGGAGAGGGAAGTGTGGGGAAGGTACAGGGGACCTCGACA
GTGAAGCATTCTGGGGTTTTCCTCCTGCATTTCGAGCTCCCCAGCCCCCAACATCTGGTTAGTCTTTAAC
TTCCTCGGGTTCATAACCATAGCAGTCCAGGAGTGGTGGGCATATTCTGTGCCCGTGGGGACCCCCGGTT
GTGTCCTGTTCGACTCAGAAGACTTGGAGAAGCCAGAGGCTGTTGGTGGGAGGGAAGTGAGGAGGGAGGA
GGGGCTGGGTGGCTGGGCCTGTGCACCCCAGCCCCTGCCCATGCCCATGCCTTGCTCTCTTTCTGTCCTC
AGTGGTCCCAGGCTGCACCCATGGCAGAAGGAGGAGGGCAGAATCATCACGAAGGTGAGTCCCCCTGGCT
GTTGGATGGGGTTCCCTGTCCTCTCAGGGGATGGGTGGATGGCCTAATTCCTTTTTCTTCAGAACTGTGG
GGAGGAAGGGGAAGGGGCACAGGAATATAAGGATCAAGAAAGAAAGAGCTGGGCACCACGAGGTTCACCC
TCAGTTTCGTGAGGACTCTCCGCTGTTCAGGTCTCTGCTAGAAGTAGGACTTGTTGCCTTTTTCTTCTGC
TCTTTCCAGTAAAATTTTATTTGGAGAAGGAGTCGTGCGCACAGAGCAGGAAGACAGTGTTCAGGGATCC
TAGGTGTTGGGGGAAGTGTCCCTTGTTTCCCCTAGCTCCCAGGGGAGAGTGGACATTTAGTGTCATTTCC
TATATAGACATGTCCCATTTGTGGGAACTGTGACCCTTCCTGTGTGAGCTGGAGGCACAGAGGGCTCAGC
CTAATGGGATCTCTCCTCCCTTCCCTGGTTTGCATTCCTTTGGGGGTGGAGAAAACCCCATTTGACTATG
TTCGGGTGCTGTGAACTTCCCTCCCAGGCCAGCAGAGGGCTGGCTGTAGCTCCCAGGCGCCCCGCCCCCC
TGCCCAACCCCGAGTCCGCCTGCCTTTTGTTCCGTTGTGGTTTGGATCCTCCCATTTCTCTGGGGACACC
CTGGCTCTCCCCACCACTGACTGTGGCCTGTGCTCTCCACCTCTGGGGAGGGAAGGCCCTGGGGTCTTCC
TTCCCGCGAGTTTCCCTGACCTAAATCTGGCGTGGCTGGGTAGTGGCCAGCAGTGGTGATGCCCAGCCTG
TTCTGCCTCCTCCTTCCCCACCCCAGGAGCCCTTTCCTTGGCCTAGGACCTGGCTTCTCAGCCACTGACC
GGCCCCCTGCTTCCAGTGCGCCACTTACCCCTTCCAGCTTCCCAGTGGTCTCTGGTCTGGGAGAGGCAGG
ACAAAGGTCTTTGTTTGCTGGAGAAAAGGTTGTCTGCGATAAATAAGGAAAACCACGAAAGCCTGGTTGT
TGGAGTGTACGTGTGTGCTCCCCCAGGCAGTGGAGGCCAGCCCTCCTTGGAGGGGCGGCTGCCTGATGAA
GGATGCGGGTGAGGTTCCCCGCCTCCACCTCCCATGGGACTTGGGGATTCATTCCAAGGGGAAGCTTTTT
GGGGGAATTCCTACCCCAGGTCTTTTTACCCTCAGTTACCAACCCCTTGCCCAGGCCAGACCTTCCTGCT
ATCCCCTCCTGGGCCACAAGCCTGGCCCTCCTCTGTCCCAATTGTGATGAAGGGGCAGTTCAAAACTTCT
TGATTAGTCATCTTCTCCCCTATCGACTTGGCTTTAAAAAATGACCTTTTCAGACTTCTAGTCTCGTTCA
CTCTTTTTGATGATGCTTTGCCGTAACCCTTCGTGGGTAGAGAAGGATTCTGTGCCCATTGGTGGTCTGG
ATAAAAGAAATAGAGACCTCACAGGAAGCAGTGGACTGGCCTGTTTCCCCACTGTTCTTTCTGTTTTCAC
ACCTGTGGCCTTCTCCCCACCTTCTTCCCAATCAACCTATTGTGTACATAGCCCCCCTCATTGTCCTTTA
TTCTTCTGGAAAGCAGACCTTGGAGGGAGGAGTGAGGGGGAGGCTCAGCTGTGGTCTCTGGGGGGTGGGG
GTTGGGAGCTGGGGTGGAAGTCCACGAAGCATACACTTAAGATGCTTTGGTGAAGTTCTAAACTTCATAT
TACCCAGGCTGAAAAAAGAGCACTTGTTCCTAGGGCTGGAAATGGAAGCCAAAACACCACCTTTTTCAGC
CTGTTTCAGCATCTTTAGAGATCAGCCCAACCCACTTACACAGTTGAGCAGAGTTGGAGGCCTAGAGAGG
GGAGGGACTGGCCCAAGGTCATACCAACTCATGGCCAGAGCCTGGGCCTCCTCACTGGCCAGGTGTTATT
TCTTCCCTCTGGGTAGGGAACCTATTTCAGGGACAGGATTGCTATGTGGTAGTGGTGGTGGGGTGCGATA
GGCGTGGCAGGCTGGGCCACAATTTGGAGTAGTCATGCCAGAGTCCTGCATTTATTTATTCTCAAGGGCC
CCGCCTCTGTGGCCCAGAATTACCCCTTCATGCTCCAGTGCACCCCAGGCTTCGTGGCCAGCCTGGGAAA
CTGTCTCTACCCTGGTCTCCCTTCAGATCAGCTTCTAGAAATGTTTCGTGGCTACAGTGGCAGCACTGTT
TTTTCCATGATGCAAGCAGTTTGCCCTCTTGGGCGGGGTTATCAGTGGCTGGCAGGGCTGGCACAGCGTG
TCCGCCCACTGCCACCTGTGGGTTCCAGGAGGGCCCAGCCCCTGTGCTGATGCCCACCACCTTCTCAGCT
CATGTCTGGGGAAGAGGACTGGCAGGGGGAAAGGTGCCTCCTCCTGAAAGGTGCCTCCTCTGTTTTTGCC
TAATATAGGCTTGGGAACACTTTGATGTCAGCTAATTCTGACTCCTTTACTTACTAGCTGTGCGGCCTTG
GGGCAACTTACTTAGCCTCTTTGAGCCTCCTGTTCCCCATCTGTAAAATGGAATCTCAATAGTGTCTAAT
AGTACCATGTGGAGAAACTTGTGTGAAATGATAGCTGTGGACTACTGTACACAGTACTCAGGATGTAGTA
AGTGCTCAATAAACAGCTGTTGGTATGGTTGACGTTATGGTAGTGGTTGTGGGGAGGACGTAGGAAACTG
GAGACTAGCTTGGCAAAGCTGGCTCTTCCTCCTTTTAGGGAAAGCTTAGAGCATCCCCATGGGGTATACC
CATACTCAGACTGTCCTCTGGCATCGAGGTTGGCCCAGGATTCAGTTCAGCTGTCACAGTGAGGTGGCGG
GATCAGATGTGGCAGGCCATGTCCCTTGGAACTTGAGTACATCGTGTGATCTCTGGAATGAAAACAGGCC
TTCACCAGTGTTGATGGTGGAAAGCTTAGGGAAGTGCTTCAAACACAGTAGGAGGGACTTACGTTAGATT
TTGGAAGGACTTGCCTGATTCGGAAGCTCCAAAGAGTGGCATTACAGAGCTGGGTGGAGAGAGGGGCTAG
CCATCTTTTGTGTCGCCCACCGGGCTCATGTGTCATCGCCTCTCATGCAGTGGTGAAGTTCATGGATGTC
TATCAGCGCAGCTACTGCCATCCAATCGAGACCCTGGTGGACATCTTCCAGGAGTACCCTGATGAGATCG
AGTACATCTTCAAGCCATCCTGTGTGCCCCTGATGCGATGCGGGGGCTGCTGCAATGACGAGGGCCTGGA
GTGTGTGCCCACTGAGGAGTCCAACATCACCATGCAGGTGGGCATCTTTGGGAAGTGGGGCAAGGGGGGG
ATAGGGAGGGGGGTAACACTTTGGGAACAGGTGGTCCCAGGTCGTTTCCTGGCTAGATTTGCCTTGTCTG
GCTCCTGCCCCTGAGTTGCACAGGGGAGGTATGGTGGGGTCTTGCCTTCTGTGGAGAAGATGCTTCATTC
CCAGCCCAGGTTCCCAGCAAGCCCCAACCATCTCCTTCTCCCTGATGGTTGCCCATGGGCTCAGGAGGGG
ACAGATGGATGCCTGTGTCAGGAGCCCCTCTCTCCCTCTCTTGGAGAGAGTCCTGAGTGCCCCCCCTTCT
TGGGGGCTTTGTTTGGGAAGCTGGATGAGCCTGGTCCATGGAGAGTTTAAAAAGTCTTTTGGTGTTACCT
GGTAATGGGGCACATCTCAGCCCAGATAGGGTGGGAGGGAGCTGTGAAACACAGGGAGGGGGTTGCTTTC
GGGTATCTACTAGGAGTCAGGGTGAAGCCTAGAGAGGATGAAAGAAGGGGAGGGGATGGGGAGTGGTAAG
AACCTAGGATTTGAATTCCCAGCCTGGCCAACCCTTGCAGCCATGTCTTGGCCTCAAGTGGAACAAGGGC
TCCTTGAGGCCAGCAGGGTTGGGGGAGTTGGGGTGGGCCTGAGCCTCTTTCCTGCTAGAGCTCTTGGTCC
TCCCTGCCTCCACCACCCATCCCTGCTCTGCAGAACCCCTGGGTGCTGAGTGGCAGGAGCCCCAGGGTTG
TCCCATCTGGGTATGGCTGGCTGGGTCACTAACCTCTGTGATCTGCTTCCTTCCTTTCCAGATTATGCGG
ATCAAACCTCACCAAGGCCAGCACATAGGAGAGATGAGCTTCCTACAGCACAACAAATGTGAATGCAGGT
GAGGATGTAGTCACGGATTCATTATCAGCAAGTGGCTGCAGGGTGCCTGATCTGTGCCAGGGTTAAGCAT
GCTGTACTTTTTGGCCCCCGTCCAGCTTCCCGCTATGTGACCTTTGGCATTTTACTTCAATGTGCCTCAG
TTTCTACATCTGTAAAATGGGCACAATAGTAGTATACTTCATAGCATTGTTATAATGATTAAACAAGTTA
TATATGAAAAGATTAAAACAGTGTTGCTCCATAATAAATGCTGTTTTTACTGTGATTATTATTGTTGTTA
TCCCTATCATTATCATCACCATCTTAACCCTTCCCTGTTTTGCTCTTTTCTCTCTCCCTACCCATTGCAG
ACCAAAGAAAGATAGAGCAAGACAAGAAAAGTAAGTGGCCCTGACTTTAGCACTTCTCCCTCTCCATGGC
CGGTTGTCTTGGTTTGGGGCTCTTGGCTACCTCTGTTGGGGGCTCCCATAGCCTCCCTGGGTCAGGGACT
TGGTCTTGTGGGGGACTTGTGGTGGCAGCAACAATGGGATGGAGCCAACTCCAGGATGATGGCTCTAGGG
CTAGTGAGAAAACATAGCCAGGAGCCTGGCACTTCCTTTGGAAGGGACAATGCCTTCTGGGTCTCCAGAT
CATTCCTGACCAGGACTTGCTGTTTCGGTGTGTCAGGGGGCACTGTGGACACTGGCTCACTGGCTTGCTC
TAGGACACCCACAGTGGGGAGAGGGAGTGGGTGGCAGAGAGGCCAGCTTTTGTGTGTCAGAGGAAATGGC
CTCTTTTGGTGGCTGCTGTGACGGTGCAGTTGGATGCGAGGCCGGCTGGAGGGTGGTTTCTCAGTGCATG
CCCTCCTGTAGGCGGCAGGCGGCAGACACACAGCCCTCTTGGCCAGGGAGAAAAAGTTGAATGTTGGTCA
TTTTCAGAGGCTTGTGAGTGCTCCGTGTTAAGGGGCAGGTAGGATGGGGTGGGGGACAAGGTCTGGCGGC
AGTAACCCTTCAAGACAGGGTGGGCGGCTGGCATCAGCAAGAGCTTGCAGGGAAAGAGAGACTGAGAGAG
AGCACCTGTGCCCTGCCCTTTCCCCCACACCATCTTGTCTGCCTCCAGTGCTGTGCGGACATTGAAGCCC
CCACCAGGCCTCAACCCCTTGCCTCTTCCCTCAGCTCCCAGCTTCCAGAGCGAGGGGATGCGGAAACCTT
CCTTCCACCCTTTGGTGCTTTCTCCTAAGGGGGACAGACTTGCCCTCTCTGGTCCCTTCTCCCCCTCCTT
TCTTCCCTGTGACAGACATCCTGAGGTGTGTTCTCTTGGGCTTGGCAGGCATGGAGAGCTCTGGTTCTCT
TGAAGGGGACAGGCTACAGCCTGCCCCCCTTCCTGTTTCCCCAAATGACTGCTCTGCCATGGGGAGAGTA
GGGGGCTCGCCTGGGCTCGGAAGAGTGTCTGGTGAGATGGTGTAGCAGGCTTTGACAGGCTGGGGAGAGA
ACTCCCTGCCAAGTACCGCCCAAGCCTCTCCTCCCCAGACCTCCTTAACTCCCACCCCATCCTGCTGCCT
GCCCAGGGCTCCAGGACACCCAGCCCTGCCTCCCAGTCCAGGTCGTGCTGAGCAGGCTGGTGTTGCTCTT
GGTTCCGTGCCAGCTCCCAAGGTAGCCGCTTCCCCCACACCGGGATTCCCAGAGGTTCTGTCGCAGTTGC
AAATGAAGGCACAAGGCCTGATACACAGCCCTCCCTCCCACTCCTGCTCCCCATCCAGGCAGGTCTCTGA
CCTTCTCCCCAAAGTCTGGCCTACCTTTTATCACCCCCGGACCTTCAGGGTCAGACTTGGACAGGGCTGC
TGGGCAAAGAGCCTTCCCTCAGGCTTTGCCCCCTGCCGGGGACTGGGAGCCACTGTGAGTGTGGAGACCT
TTGGGTCCTGTGCCCTCCACCCAGTCTCGGCTTCCCACCAAAGCCTTGTCAGGGGCTGGGTTTGCCATCC
CATGGTGGGCAGCGTGAGGAGAAGAAAGAGCCATCGAGTGCTTGCTGCCCAGACACGCCTGTGTGCGCCC
GCGCATGCCTCCCCAGAGACCACCTGCCTCCTGACACTTCCTCCGGGAAGCGGCCCTGTGTGGCTTTGCT
TTGGTCGTTCCCCCATCCCTGCCCACCTTACCACTTCTTTTACTCCCCCCACCGCCCCCGCTCTCTCTCT
GTCTCTGTTTTTTTATTTTCCAGAAAATCAGTTCGAGGAAAGGGAAAGGGGCAAAAACGAAAGCGCAAGA
AATCCCGGTATAAGTCCTGGAGCGTGTACGTTGGTGCCCGCTGCTGTCTAATGCCCTGGAGCCTCCCTGG
CCCCCAGTACAACCTCCGCCTGCCATTCCCTGTAACCCTGCCTCCCTCCCCTGGTCCTTCCCTGGCTCTC
ATCCTCCTGGCCCGTGTCTCTCTCTCACTCTCTCACTCCACTAATTGGCACCAACGGGTAGATTTGGTGG
TGGCATTGCTGGTCCAGGGTTGGGGTGAATGGGGGTGCCGACTTGGCCTGGAGGATTAAGGGAGGGGACC
CTGGCTTGGCTGGGCACCGATTTTCTCTCACCCACTGGGCACTGGTGGCGGGCCCATGTTGGCACAGGTG
CCTGCTCACCCAACTGGTTTCCATTGCTCTAGGCTTCTGCACTCGTCTGGAAGCTGAGGGTGGTGGGGAG
GGCAGACATGGCCCAAGAAGGGCTGTGAATGACTGGAGGCAGCTTGCTGAATGACTCCTTGGCTGAAGGA
GGAGCTTGGGTGGGATCAGACACCATGTGGCGGCCTCCCTTCATCTGGTGGAAGTGCCCTGGCTCCTCAC
GGAGGTGGGGCCTCTGGAGGGGAGCCCCCTATTCCGGCCCAACCCATGGCACCCACAGAGGCCTCCTTGC
AGGGCAGCCTCTTCCTCTGGGTCGGAGGCTGTGGTGGGCCCTGCCCTGGGCCCTCTGGCCACCAGCGGCC
TGGCCTGGGGACACCGCCTCCGGGCTTAGCCTCCCATCACACCCTACTTTAGCCCACCTTGGTGGAAGGG
CCTGGACATGAGCCTTGCACGGGGAGAAGGTGGCCCCTGATTGCCATCCCCAGCAGGTGAAGAGTCAAGG
CGTGCTCCGATGGGGGCAACAGCAGTTGGGTCCCTGTGGCCTGAGACTCACCCTTGTCTCCCAGAGACAC
AGCATTGCCCCTTATGGCAGCCTCTCCCTGCACTCTCTGCCCGTCTGTGCCCGCCTCTTCCTGCGGCAGG
TGTCCTAGCCAGTGCTGCCTCTTTCCGCCGCTCTCTCTGTCTTTTGCTGTAGCGCTCGGATCCTTCCAGG
GCCTGGGGGCTGACCGGCTGGGTGGGGGTGCAGCTGCGGACATGTTAGGGGGTGTTGCATGGTGATTTTT
TTTCTCTCTCTCTGCTGATGCTCTAGCTTAGATGTCTTTCCTTTTGCCTTTTTGCAGTCCCTGTGGGCCT
TGCTCAGAGCGGAGAAAGCATTTGTTTGTACAAGATCCGCAGACGTGTAAATGTTCCTGCAAAAACACAG
ACTCGCGTTGCAAGGCGAGGCAGCTTGAGTTAAACGAACGTACTTGCAGGTTGGTTCCCAGAGGGCAAGC
AAGTCAGAGAGGGGCATCACACAGAGATGGGGAGAGAGAGAGAGAAAGAGAGTGAGCGAGCGAGCGAGCG
GGAGAGCGCCTGAGAGGGGCCAGCTGCTTGCTCAGTTTCTAGCTGCCTGCCTGGTGACTGCTGCCTTCTC
TGCTTTTAAGGCCCCTGTGGTGGGCTGCAGGCACTGGTCCAGCCTGGCGGGGCCTGTTCCGAGGTTGCCC
TGGTTGCCTGAGTGGTAGGCTGGTGTGGCTTAGTGTAGTGGTGTGGACGCAAGCTGTGTGTTGTGTCCTG
TGGTCCTTCTGCTCATAGTGGCTGTTGGTCCTGATGTTATTACTACCTCTGGTAGTAATGCTGAGAAGCT
GAAAGCCGATTCCAGGTGTGGACAATGTCAACAAAGCACAGATGCTCTCGCTGGGGCCTTGCCTCGGCCC
TTTGAAGTCTGCATGGCTGGGCTTCTCACTCACTCAGTGTTTCTTGCTGGGGGAAGGAATTGAGTCTCCC
ACTTCAGACTGGGCCTCCCTGAGGAAAGGGTTGTGTCTCCCCACTCAGACTGAGGTTCCCTGAGGGTAGG
GCTGTGTCTCTCCCCTCCGACCTGGGCTCCCTGATAGGGCTGTCTCCCCGCTCAGACTGAGGCTCCCTCA
GGCCAGGGCTATGTCTCCCTCCTCAGACTGGGGCTCTGAGGGCAAGGGGTCTGGCTGTTCGTTTAGGATG
GGGCACTTTTGCCTACACACTGAAGGAGCTGTAGCATCCAAGAATACTAGATACCTTTAATCCTCCACCA
GTCATGGTGACAACCCCAAGCAGCCCACACATTTTCAAGTGCCCCCAGGATGCGTGGAGGGAGGGGTCTG
TGCCCATTCTCCTGACATTAGCCTGTGAGCTCCGTAAGCCCGGGCCTCGTTTACGTACCTTTGTGAGCCC
CGGGCATCTGTACCTCTTTCCTTTGCCCATACTGGGGACCAAGGAAGTGTCAAGTGCATGAGTGAATGTG
TGACTCAGTTCAGAGGGTGAGGTCAGGAGCACAGGGTCGGGACAGGTGGCTGGCATCTTTTAATGCCTTA
GCTTATGTTCTTTATACCAACTTGGCCTGTGCTCAGAGTGAGGGAGGCCCTGGGGGTCAGGGTAAGCGTC
AGTCAGGGAGGCAAGACTTTGTGGGGATTTCCTAGACAGGGCCAAGGCACCCCCAGCTCACCCCGAGGCT
GTGTTAGGGAAGTCCTTGGAGTGTCTCCCCTCCCCCAGCAATGTTCTTGTGGCTTGTGTGTGCTCAGGGG
ATGCTGGGAACCAGGCCTGGGTAGTTGGTGTGGGGTGCTGTCTGTCTTGGCCCTATGTGAAACCAAGAGG
GCGTATATTAGTGCTGGGGTGGGGGCTCTGCCTAACTTCAGGGCTGGATGAGGGGAGTCTCAGTTCCCCA
GGGGTCCTTGGGAAAGATAAGGGACTTGACATTTTAGGGTTTTTAGGTGATTATTCTGCTGATGGGGGTT
TGTGTGAAGTGACCTGGGAGCTAACTGAAGTTACTCTAACCTCCCAATACCTTTACCCAACCCCCAAGCT
GGCTGTATCTGGGAATATCAGTTTCCAAAATTGGAGGCTTAGGACTCCGTTTCGGGGCTCCCCAGAAGGG
TAGGGCCTGTTCTGCCTCCTTCTCACAATCACCCAGGGGCAGGGGCATGCTGAGAAAGTTCTTGGAGGCC
CCCTTTGCTTCAGCTGGAGTAGTGAAGCCGCCGAATTGTCTCTCCCCATCCTAAGTGAAGCAGCATATTT
GAAAGGAAAGACAACCTGTTACCTGGGCCTGCAACCTCCAGGCAGCTCAAGAGAGATGAGGCCTACAGCC
ACAGTGGGAGGGGACATGGGGAATGGAGATGGTCCCTCACCTTCCTGGGGCCTCCTGCTCTACGCTACCC
CCTCGGGAGCCTCCTGTCCCCAGGGCAGGCCCTTGCCATTGTTGGTCACCCGGCCAAGCCTCTCTGCCTC
AGGCGTTCTCCCAGAAGATCTGCCCACTCTCTTCCCCACACCAGCCCCTAGAGACTGAACTGAAAACCCT
CCTCAGCAGGGAGCCTCTTCTGATTAACTTCATCCAGCTCTGGTCACCCATCAGCTCTTAAAATGTCAAG
TGGGGACTGTTCTTTGGTATCCGTTCATTTGTTGCTTTGTAAAGTGTTCCCATGTCCTTGTCTTGTCTCA
AGTAGATTGCAAGCTCAGGAGGGTAGACTGGGAGCCCCTGAGTGGAGCTGCTGCTCAGGCCGGGGCTCCC
TGAGGGCAGGGCTGGGGCTGTTCTCATACTGGGGCTTTCTGCCCCAGGACCACACCTTCCTGTCCTCTCT
GCTCTTATGGTGCCGGAGGCTGCAGTGACCCAGGGGCCCCCAGGAATGGGGAGGCCGCCTGCCTCATCGC
CAGGCCTCCTCACTTGGCCCTAACCCCAGCCTTTGTTTTCCATTTCCCTCAGATGTGACAAGCCGAGGCG
GTGAGCCGGGCAGGAGGAAGGAGCCTCCCTCAGGGTTTCGGGAACCAGATCTCTCACCAGGAAAGACTGA
TACAGAACGATCGATACAGAAACCACGCTGCCGCCACCACACCATCACCATCGACAGAACAGTCCTTAAT
CCAGAAACCTGAAATGAAGGAAGAGGAGACTCTGCGCAGAGCACTTTGGGTCCGGAGGGCGAGACTCCGG
CGGAAGCATTCCCGGGCGGGTGACCCAGCACGGTCCCTCTTGGAATTGGATTCGCCATTTTATTTTTCTT
GCTGCTAAATCACCGAGCCCGGAAGATTAGAGAGTTTTATTTCTGGGATTCCTGTAGACACACCCACCCA
CATACATACATTTATATATATATATATTATATATATATAAAAATAAATATCTCTATTTTATATATATAAA
ATATATATATTCTTTTTTTAAATTAACAGTGCTAATGTTATTGGTGTCTTCACTGGATGTATTTGACTGC
TGTGGACTTGAGTTGGGAGGGGAATGTTCCCACTCAGATCCTGACAGGGAAGAGGAGGAGATGAGAGACT
CTGGCATGATCTTTTTTTTGTCCCACTTGGTGGGGCCAGGGTCCTCTCCCCTGCCCAGGAATGTGCAAGG
CCAGGGCATGGGGGCAAATATGACCCAGTTTTGGGAACACCGACAAACCCAGCCCTGGCGCTGAGCCTCT
CTACCCCAGGTCAGACGGACAGAAAGACAGATCACAGGTACAGGGATGAGGACACCGGCTCTGACCAGGA
GTTTGGGGAGCTTCAGGACATTGCTGTGCTTTGGGGATTCCCTCCACATGCTGCACGCGCATCTCGCCCC
CAGGGGCACTGCCTGGAAGATTCAGGAGCCTGGGCGGCCTTCGCTTACTCTCACCTGCTTCTGAGTTGCC
CAGGAGACCACTGGCAGATGTCCCGGCGAAGAGAAGAGACACATTGTTGGAAGAAGCAGCCCATGACAGC
TCCCCTTCCTGGGACTCGCCCTCATCCTCTTCCTGCTCCCCTTCCTGGGGTGCAGCCTAAAAGGACCTAT
GTCCTCACACCATTGAAACCACTAGTTCTGTCCCCCCAGGAGACCTGGTTGTGTGTGTGTGAGTGGTTGA
CCTTCCTCCATCCCCTGGTCCTTCCCTTCCCTTCCCGAGGCACAGAGAGACAGGGCAGGATCCACGTGCC
CATTGTGGAGGCAGAGAAAAGAGAAAGTGTTTTATATACGGTACTTATTTAATATCCCTTTTTAATTAGA
AATTAAAACAGTTAATTTAATTAAAGAGTAGGGTTTTTTTTCAGTATTCTTGGTTAATATTTAATTTCAA
CTATTTATGAGATGTATCTTTTGCTCTCTCTTGCTCTCTTATTTGTACCGGTTTTTGTATATAAAATTCA
TGTTTCCAATCTCTCTCTCCCTGATCGGTGACAGTCACTAGCTTATCTTGAACAGATATTTAATTTTGCT
AACACTCAGCTCTGCCCTCCCCGATCCCCTGGCTCCCCAGCACACATTCCTTTGAAATAAGGTTTCAATA
TACATCTACATACTATATATATATTTGGCAACTTGTATTTGTGTGTATATATATATATATATGTTTATGT
ATATATGTGATTCTGATAAAATAGACATTGCTATTCTGTTTTTTATATGTAAAAACAAAACAAGAAAAAA
TAGAGAATTCTACATACTAAATCTCTCTCCTTTTTTAATTTTAATATTTGTTATCATTTATTTATTGGTG
CTACTGTTTATCCGTAATAATTGTGGGGAAAAGATATTAACATCACGTCTTTGTCTCTAGTGCAGTTTTT
CGAGATATTCCGTAGTACATATTTATTTTTAAACAACGACAAAGAAATACAGATATATCTTAAAAAAAAA
AAAGCATTTTGTATTAAAGAATTTAATTCTGATCTCAAA
SEQ ID NO: 4
>gi|559098479|ref|NM_001287044.1| Homo sapiens vascular
endothelial growth factor A (VEGFA), transcript variant
10, mRNA
AGCCCGGGCCTGGCCGGCCGCGTGTTCCCGGAGCCTCGGCTGCCCGAATGGGGAGCCCAGAGTGGCGAGC
GGCACCCCTCCCCCCGCCAGCCCTCCGCGGGAAGGTGACCTCTCGAGTGGTCCCAGGCTGCACCCATGGC
AGAAGGAGGAGGGCAGAATCATCACGAAGTGGTGAAGTTCATGGATGTCTATCAGCGCAGCTACTGCCAT
CCAATCGAGACCCTGGTGGACATCTTCCAGGAGTACCCTGATGAGATCGAGTACATCTTCAAGCCATCCT
GTGTGCCCCTGATGCGATGCGGGGGCTGCTGCAATGACGAGGGCCTGGAGTGTGTGCCCACTGAGGAGTC
CAACATCACCATGCAGATTATGCGGATCAAACCTCACCAAGGCCAGCACATAGGAGAGATGAGCTTCCTA
CAGCACAACAAATGTGAATGCAGACCAAAGAAAGATAGAGCAAGACAAGAAAATCCCTGTGGGCCTTGCT
CAGAGCGGAGAAAGCATTTGTTTGTACAAGATCCGCAGACGTGTAAATGTTCCTGCAAAAACACAGACTC
GCGTTGCAAGGCGAGGCAGCTTGAGTTAAACGAACGTACTTGCAGATGTGACAAGCCGAGGCGGTGAGCC
GGGCAGGAGGAAGGAGCCTCCCTCAGGGTTTCGGGAACCAGATCTCTCACCAGGAAAGACTGATACAGAA
CGATCGATACAGAAACCACGCTGCCGCCACCACACCATCACCATCGACAGAACAGTCCTTAATCCAGAAA
CCTGAAATGAAGGAAGAGGAGACTCTGCGCAGAGCACTTTGGGTCCGGAGGGCGAGACTCCGGCGGAAGC
ATTCCCGGGCGGGTGACCCAGCACGGTCCCTCTTGGAATTGGATTCGCCATTTTATTTTTCTTGCTGCTA
AATCACCGAGCCCGGAAGATTAGAGAGTTTTATTTCTGGGATTCCTGTAGACACACCCACCCACATACAT
ACATTTATATATATATATATTATATATATATAAAAATAAATATCTCTATTTTATATATATAAAATATATA
TATTCTTTTTTTAAATTAACAGTGCTAATGTTATTGGTGTCTTCACTGGATGTATTTGACTGCTGTGGAC
TTGAGTTGGGAGGGGAATGTTCCCACTCAGATCCTGACAGGGAAGAGGAGGAGATGAGAGACTCTGGCAT
GATCTTTTTTTTGTCCCACTTGGTGGGGCCAGGGTCCTCTCCCCTGCCCAGGAATGTGCAAGGCCAGGGC
ATGGGGGCAAATATGACCCAGTTTTGGGAACACCGACAAACCCAGCCCTGGCGCTGAGCCTCTCTACCCC
AGGTCAGACGGACAGAAAGACAGATCACAGGTACAGGGATGAGGACACCGGCTCTGACCAGGAGTTTGGG
GAGCTTCAGGACATTGCTGTGCTTTGGGGATTCCCTCCACATGCTGCACGCGCATCTCGCCCCCAGGGGC
ACTGCCTGGAAGATTCAGGAGCCTGGGCGGCCTTCGCTTACTCTCACCTGCTTCTGAGTTGCCCAGGAGA
CCACTGGCAGATGTCCCGGCGAAGAGAAGAGACACATTGTTGGAAGAAGCAGCCCATGACAGCTCCCCTT
CCTGGGACTCGCCCTCATCCTCTTCCTGCTCCCCTTCCTGGGGTGCAGCCTAAAAGGACCTATGTCCTCA
CACCATTGAAACCACTAGTTCTGTCCCCCCAGGAGACCTGGTTGTGTGTGTGTGAGTGGTTGACCTTCCT
CCATCCCCTGGTCCTTCCCTTCCCTTCCCGAGGCACAGAGAGACAGGGCAGGATCCACGTGCCCATTGTG
GAGGCAGAGAAAAGAGAAAGTGTTTTATATACGGTACTTATTTAATATCCCTTTTTAATTAGAAATTAAA
ACAGTTAATTTAATTAAAGAGTAGGGTTTTTTTTCAGTATTCTTGGTTAATATTTAATTTCAACTATTTA
TGAGATGTATCTTTTGCTCTCTCTTGCTCTCTTATTTGTACCGGTTTTTGTATATAAAATTCATGTTTCC
AATCTCTCTCTCCCTGATCGGTGACAGTCACTAGCTTATCTTGAACAGATATTTAATTTTGCTAACACTC
AGCTCTGCCCTCCCCGATCCCCTGGCTCCCCAGCACACATTCCTTTGAAATAAGGTTTCAATATACATCT
ACATACTATATATATATTTGGCAACTTGTATTTGTGTGTATATATATATATATATGTTTATGTATATATG
TGATTCTGATAAAATAGACATTGCTATTCTGTTTTTTATATGTAAAAACAAAACAAGAAAAAATAGAGAA
TTCTACATACTAAATCTCTCTCCTTTTTTAATTTTAATATTTGTTATCATTTATTTATTGGTGCTACTGT
TTATCCGTAATAATTGTGGGGAAAAGATATTAACATCACGTCTTTGTCTCTAGTGCAGTTTTTCGAGATA
TTCCGTAGTACATATTTATTTTTAAACAACGACAAAGAAATACAGATATATCT
* * *

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for determining a genotype for a subject, comprising: determining a genotype of one or more genetic marker alleles at one or more genetic marker loci associated with (i) a level of ocular VEGF and/or (ii) a VEGF suppression response to an anti-VEGF treatment (e.g., VEGF suppression time), for nucleic acid from a subject.

2. The method of claim 1, wherein the subject has been observed to have one or more indicators of wet age-related macular degeneration (AMD).

3. The method of claim 1, wherein the subject has been observed to have one or more indicators of choroidal neovascularization (CNV).

4. The method of claim 1, wherein the one or more genetic marker alleles are associated with an ocular VEGF suppression response to a treatment that suppresses ocular VEGF.

5. The method of claim 4, wherein the VEGF suppression response is a VEGF suppression time.

6. The method of claim 1, wherein the genotype comprises two or more alleles for each of the one or more genetic marker loci.

7. The method of claim 1, wherein:
the one or more genetic marker loci comprise a single-nucleotide polymorphism (SNP) locus or SNP loci, and
the SNP locus or SNP loci are chosen from rs1870377, rs2071559, rs3025033, rs3025039, rs2305948, a SNP allele in linkage disequilibrium with an allele of one or more of the foregoing SNP loci, a SNP allele in a polynucleotide that encodes a polypeptide in a VEGF signaling pathway, a SNP allele in a first polynucleotide in operable connection with a second polynucleotide that encodes a polypeptide in a VEGF signaling pathway, or combination thereof.

8. The method of claim 1, wherein:
the one or more genetic marker loci comprise single-nucleotide polymorphism (SNP) loci, and
the genotype comprises one or more single-nucleotide polymorphism (SNP) alleles at each of the SNP loci comprising rs1870377 and rs2071559.

9. The method of claim 7, wherein a SNP allele in linkage disequilibrium with another SNP allele is characterized as having a D-prime assessment of linkage disequilibrium of 0.6 or greater.

10. The method of claim 1, which comprises predicting for the subject, according to the genotype, a VEGF suppression response to a treatment that suppresses a VEGF, thereby providing a VEGF suppression prediction.

11. The method of claim 10, wherein the prediction comprises a VEGF suppression time prediction.

12. The method of claim 11, wherein a genotype comprising two alleles of rs1870377 is determined, and a VEGF suppression time predicted for a genotype comprising homozygous thymine alleles is longer than a VEGF suppression time predicted for a genotype comprising heterozygous adenine and thymine alleles.

13. The method of claim 11, wherein a genotype comprising two alleles of rs1870377 is determined, and a relatively high VEGF suppression time is predicted for a genotype comprising homozygous thymine alleles.

14. The method of claim 11, wherein a genotype comprising two alleles of rs2071559 is determined, and a VEGF suppression time predicted for a genotype comprising heterozygous guanine and adenine alleles is longer than (i) a VEGF suppression time predicted for a genotype comprising homozygous adenine alleles, and (ii) a VEGF suppression time predicted for a genotype comprising homozygous guanine alleles.

15. The method of claim 11, wherein a genotype comprising two alleles of rs1870377 and two alleles of rs2071559 is determined, and
(i) a VEGF suppression time predicted for a genotype comprising heterozygous guanine and adenine alleles for rs2071559 and homozygous thymine alleles for rs1870377,
is longer than
(ii) a VEGF suppression time predicted for a genotype comprising homozygous guanine or adenine alleles for rs2071559 and homozygous adenine alleles or heterozygous adenine and thymine alleles for rs1870377.

16. The method of claim 11, wherein a genotype comprising two alleles of rs1870377 and two alleles of rs2071559 is determined, and a relatively long VEGF suppression time is predicted for a genotype comprising heterozygous guanine and adenine alleles for rs2071559 and homozygous thymine alleles for rs1870377.

17. The method of claim 11, wherein a genotype comprising two alleles of rs1870377 and two alleles of rs2071559 is determined, and a relatively short VEGF suppression time is predicted for a genotype comprising homozygous guanine or adenine alleles for rs2071559 and homozygous adenine alleles or heterozygous adenine and thymine alleles for rs1870377.

18. The method of claim 10, which comprises selecting a dosing interval for the treatment according to the prediction.

19. The method of claim 18, wherein the dosing interval selected is less than or equal to the suppression time prediction for the subject.

20. The method of claim 10, which comprises selecting a treatment of the AMD according to the prediction.

21. The method of claim 1, wherein the ocular VEGF is retinal VEGF.
